(19) United States
(12) Patent Application Publication
(10) Pub. No.: US 2021/0253686 A1
(43) Pub. Date: Aug. 19, 2021

(54) METHODS FOR THE TREATMENT OF SCLERODERMA AND RELATED CONDITIONS

(71) Applicant: HZNP Limited, Hamilton Pembroke (BM)

(72) Inventors: Elizabeth THOMPSON, Lake Forest, IL (US); Srini RAMANATHAN, Lake Forest, IL (US)

(21) Appl. No.: 17/168,055

(22) Filed: Feb. 4, 2021

Related U.S. Application Data
(60) Provisional application No. 63/049,522, filed on Jul. 8, 2020.
(60) Provisional application No. 62/970,063, filed on Feb. 4, 2020.

(51) Int. Cl. C07K 16/22; A61K 45/06; A61P 11/00

(52) U.S. Cl. CPC C07K 16/22; A61P 11/00; A61K 45/06

(57) ABSTRACT

Provided herein are antibodies against insulin-like growth factor 1 receptor (IGF-1R) and their use in methods of treatment of, and achievement of clinical outcomes in, scleroderma and forms thereof, including diffuse cutaneous systemic sclerosis.

This application claims the benefit of U.S. Provisional Applications No. 62/970,063, filed Feb. 4, 2020, and 63/049,522, filed Jul. 8, 2020, the disclosures of which are incorporated by reference as if written herein in their entireties.

### SEQUENCE LISTING

The instant application contains a Sequence Listing which has been submitted electronically in ASCII format and is hereby incorporated by reference in its entirety. Said ASCII copy, created on Apr. 30, 2021, is named “20210430_HOR0119-201-US_Sequence_Listing_ST25” is 78,917 bytes in size, and is incorporated by references as if written herein in its entirety.

Scleroderma is a chronic connective tissue disease and is generally classified as an autoimmune rheumatic disease. The cause of scleroderma is unknown, but evidence is mounting that permissive variations in several genes may predispose people to scleroderma.

Localized scleroderma, also known as localized fibrosing scleroderma, is limited to scleroderma that affects the skin of a patient, except in rare forms where it may reach the muscle, and has at least two broad sub-classifications, morphea and linear scleroderma, and the two are not mutually exclusive. The areas usually affected are the arms, hands, legs, feet and head. Morphea appears as waxy patches of varying sizes, shapes, and colors. The skin under these patches may thicken over time, limiting use of the nearby joint(s). Linear scleroderma forms a line of hardened skin often deeper than the morphea and usually occurs on the arms, legs, or head. Localized scleroderma can occur in childhood or in adults between the ages 30 and 50 years of age and the patches can last for 6 months to several years.

Systemic scleroderma affects the connective tissue of many parts of the body, including skin, esophagus, gastrointestinal tract, lung, kidney, heart, and other internal organs. It may indirectly affect blood vessels, muscles and joints. There are three forms of systemic scleroderma, limited, sine, and diffuse. Limited cutaneous systemic scleroderma, also known as limited cutaneous systemic sclerosis, affects the lower arms and legs and over time can affect the digestive system, lungs, heart and kidneys. Systemic sclerosis sine scleroderma, also known as limited systemic sclerosis and progressive systemic sclerosis sine scleoderma, affects one or more internal organs but not the skin. Diffuse cutaneous systemic scleroderma, also known as limited cutaneous systemic sclerosis or diffuse cutaneous systemic sclerosis, progresses much faster than limited scleroderma. Diffuse cutaneous systemic scleroderma affects all the same areas of the body as limited cutaneous systemic scleroderma, but skin hardening may occur on the trunk, uppers legs and arms, too. Systemic scleroderma onset often happens in adults between the ages 30 and 50 years of age. Systemic scleroderma often ends in death caused by fibrosis of one or more internal organs, typically the lungs.

Systemic scleroderma with fibrosis of the lungs is grouped within the broader interstitial lung disease (ILD). Interstitial lung disease encompasses a large and diverse group of parenchymal lung disorders. ILD may be classified according to the cause, for example and without limitation: inhaled substances, including silicosis, asbestosis, berylliosis and hypersensitivity pneumonitis; drug induced, such as from antibiotics, chemotherapeutic drugs (e.g., bleomycin) and antiarrhythmic agents; connective tissue disease, such as systemic sclerosis, dermatomyositis, systemic lupus erythematosus and rheumatoid arthritis; infection, such as atypical pneumonia, pneumocystis pneumonia (PCP) and tuberculosis; idiopathic, such as sarcoidosis, idiopathic pulmonary fibrosis (IPF) and Hamman-Rich syndrome; or malignancy, such as lymphangitic carcinomatosis.

IPF is one of the more studied fibrosing ILD's in the literature. Several papers suggest a role for IGF-R1 in the fibrosis of lung tissue in IPF. Upregulation of IGF1 expression and/or signaling is present in patients with ILD diseases, including IPF, systemic scleroderma ILD, late stage sarcoidosis, pneumoconiosis, drug-induced pulmonary fibrosis, rheumatoid arthritis-related interstitial lung disease. Another study showed that inhibition of the IGF-1R signaling in a SCID/Bg model of human IPF was effective in reducing profibrotic mediators. This and many other studies suggest the treatment of ILD's, including IPF and SSc ILD, with an inhibitor of IGF-1R could be beneficial.

Scleroderma affects about 300,000 people in America with about a third of those having systemic scleroderma. There are two new systemic scleroderma cases per 100,000 people per year in the US and the rate has been increasing of the last 50 years and three new linear scleroderma cases per 100,000 people in the US per year. It affects women at a rate four times more than men. Scleroderma is usually is diagnosed in adults between the ages 30 and 50 years of age, although diagnosis is often hard as it initially presents as many other types of autoimmune diseases.

IPF affects about 100,000 people in America with 30,000 to 40,000 new cases per year. There are between 13-20 new cases per 100,000 people per year in the world. IPF is usually found in people over the age of 60 although onset can occur earlier and is almost evenly split between men and women.

Currently the treatment for all forms of scleroderma is to manage the symptoms and try and slow the progression of the disease. There are currently no treatments to cure any form of scleroderma.

Treatment for localized scleroderma can vary depending upon the severity of the disease. For most morphea scleroderma patients a topical cream to keep the skin soft and pliable is all the treatment that is required. In cases where this treatment is inadequate, additional treatment with one or a combination of the following drugs can be used: topical steroids, methotrexate (Trexall), and corticosteroids. Light therapy has been shown to help in softening the skin lesions of scleroderma. Treatment for linear scleroderma includes all the above but also can include physical therapy and/or surgery depending upon the location and severity of the scleroderma. All these treatments help reduce the symptom of scleroderma, but do not cure the underlying cause of the disease.

The treatments for systemic scleroderma are dependent on the symptoms presented by the individual patient. The European League against Rheumatism (EULAR) has 16 different treatment recommendations based on symptoms presented by the patient. Medication often used for the treatment of scleroderma symptoms included proton pump inhibitors, angiotensin converting enzyme (ACE) inhibitors, calcium channel blockers, endothelin receptor blockers, prostaglandin analogues, phosphodiesterase inhibitors, and immune modulators (mycophenolate mofetil, cyclophosphamide, methotrexate). Again, these therapies only treat the symptoms, they do not stop or diminish the tissue fibrosis which is the underlying issue with systemic scleroderma.

The current understanding of the specific underlying pathogenic autoimmune mechanisms for scleroderma are incomplete. Many mechanisms are involved in the disease and which ones to target are still under study. Many of the drugs, including biologicals, for the treatment of other autoimmune disease have been tried and used for the treatment of scleroderma. However, most of these have only provided minor benefits to the subjects.

Clinical studies with Tocilizumab, a humanized anti-IL-6 receptor antibody, have shown some benefits to scleroderma subjects. Subjects have shown improvement in the modified Rodnan total skin score (mRSS or mRTSS) and the halting of lung fibrosis in some studies. Other studies have shown little improvement in lung function, but improvement in the mRSS or 28-joint count Disease Activity score. There is currently a phase III study under way for the use of Tocilizumab for the treatment of systemic scleroderma.

Clinical studies with a therapy used to block TNF-alpha have shown little benefit to scleroderma subjects. Infliximab, a humanized anti-TNF-alpha receptor mouse antibody, showed minor improvement in mRSS scores, but had major adverse events in 7 of the 16 subjects in the study. Due to this and other results with therapies used to block TNF-alpha most experts do not recommend this treatment for scleroderma.

Clinical studies of two anti-TGF-beta antibodies showed mixed results in scleroderma trials. CAT-19, a recombinant human antibody that binds only TGF-beta1 with a dissociation constant of 150 nM, shown no change in mRSS scores of the patients. A 2014 clinical study with fresolimumab, a high affinity human monoclonal antibody against all three TGF-beta isoforms, showed improvement in mRSS scores and downregulation of TGF-beta biomarkers in the skin. No further studies have been started or completed on anti-TGF-beta antibodies, and none are approved to treat scleroderma subjects.

Clinical studies of rituximab, an anti-CD20 chimeric monoclonal antibody, have been completed. These studies have shown improvement in mRSS scores, but no improvement in lung function in a forced vital capacity test. There is some interest in the use of Rituximab as an alternative to cyclophosphamide for the treatment of scleroderma.

Taking all these studies into account there is still a need for new therapies to treat scleroderma. Additional therapies to treat the symptoms of scleroderma would be useful, but a therapy which can reduce and repair the damage in scleroderma would be even more beneficial for patients, for example a drug that can reduce the fibrosis in scleroderma patients and cause the apoptosis of the cells responsible for the disease.

Insulin-like growth factor proteins are essential in regulating cell growth and death. There are three types of proteins involved in the IGF system, including two insulin-like growth factor ligands (IGF1 and IGF2), two insulin-like growth factor receptors (IGF-1R and IGF-2R), and 6 insulin-like growth factor binding proteins (IGFBP1-6). There are indications that IGF system plays a role in many forms of cancer and many autoimmune diseases. In these diseases there is a higher level of IGF present in the tissue and/or blood of the patients. Studies have shown a higher levels of IGF in the skin and blood of scleroderma patients. It is believed the higher level of IGF plays a role in preventing the normal apoptosis process of the cells involved. Therefore, stopping the overstimulation IGF pathway is expected to be beneficial to scleroderma patients.

### BRIEF DESCRIPTION OF THE DRAWINGS

FIG. 1 shows an overview of the study design disclosed herein.

### DETAILED DESCRIPTION

Provided herein are antibodies against insulin-like growth factor 1 receptor (IGF-1R) for the use in the treatment of scleroderma.

In some embodiments, the scleroderma is chosen from localized scleroderma and systemic scleroderma. In some embodiments, the scleroderma is localized scleroderma. In some embodiments, the scleroderma is systemic scleroderma.

Also provided herein is a method for reducing the collagen production and/or accumulation and fibrosis in a subject with scleroderma comprising administering to said subject an effective amount of antibody, or antigen fragment thereof, wherein said antibody specifically binds to and inhibits insulin-like growth factor 1 receptor.

Also provided herein is a method for the reducing the modified Rodnan total skin score (mRSS or mRTSS) in a subject with scleroderma (e.g. localized) comprising administering to said subject an effective amount of antibody, or antigen fragment thereof, wherein said antibody specifically binds to and inhibits insulin-like growth factor 1 receptor.

In some embodiments, the reduction in the modified Rodnan total skin score could be greater than 2, for example 3, 4, 5, 6, 7, 8, 9, 10, 11, 12, 13, 14, 15, or more than 15.

In some embodiments, the reduction is in calcification within the skin as determined by a skin biopsy.

Also provided herein is a method of reducing the collagen production and/or accumulation and fibrosis in a subject with systemic scleroderma comprising administering to said subject an effective amount of antibody, or antigen fragment thereof, wherein said antibody specifically binds to and inhibits insulin-like growth factor 1 receptor.

In some embodiments, the American College of Rheumatology-Composite Response Index in Systemic Sclerosis (ACR-CRISS) score increases by or greater than 0.1, for example 0.1, 0.11, 0.12, 0.13, 0.14, 0.15, 0.16, 0.17, 0.18, 0.19, 0.20, or more than 0.20.

Also provided herein is a method of reducing the pulmonary fibrosis in a subject with systemic scleroderma comprising administering to said subject an effective amount of antibody, or antigen fragment thereof, wherein said antibody specifically binds to and inhibits insulin-like growth factor 1 receptor.

In some embodiments, the 6-minute walk test (6MWT) distance in meters walked improves about 5 meters, about 10 meters, about 15 meters, about 20 meters, about 25 meters, about 30 meters, about 35 meters, about 40 meters, about 45 meters, or about 50 meters. In some embodiments, the increase in meters walked is from about 5 meters to about 25 meters, or from about 5 meters to about 30 meters, or from about 5 meters to about 40 meters, of from about 5 meters to about 50 meters.

Also provided herein is a method for reducing the collagen production and accumulation in a subject with localized scleroderma or systemic scleroderma comprising administering to said subject an effective amount of antibody, or antigen fragment thereof, wherein said antibody specifically binds to and inhibits insulin-like growth factor 1 receptor and is delivered in a topical formulation.

In some embodiments, the delivery is by a cream, an ointment, a patch, or any other method to deliver the effective amount of antibody, or antigen fragment thereof, to the patient though the skin.

Also provided herein is a method for reducing the collagen production and accumulation in a subject with localized scleroderma or systemic scleroderma comprising administering to said subject an effective amount of antibody, or antigen fragment thereof, wherein said antibody specifically binds to and inhibits insulin-like growth factor 1 receptor and is delivered by intradermal injections.

Also provided herein is a method for reducing the collagen production and accumulation in a subject with localized scleroderma or systemic scleroderma comprising administering to said subject an effective amount of antibody, or antigen fragment thereof, wherein said antibody specifically binds to and inhibits insulin-like growth factor 1 receptor and is delivered by subcutaneous injections.

Also provided herein is a method for reducing the collagen production and accumulation in a subject with localized scleroderma or systemic scleroderma comprising administering to said subject an effective amount of antibody, or antigen fragment thereof, wherein said antibody specifically binds to and inhibits insulin-like growth factor 1 receptor and is delivered by inhalation.

In some embodiments, the delivery of the effective amount of antibody, or antigen fragment thereof is by an inhaler.

In some embodiments, the delivery of the effective amount of antibody, or antigen fragment thereof is by a nebulizer.

Also provided herein is a method for reducing the collagen production and accumulation in a subject with localized scleroderma or systemic scleroderma comprising administering to said subject an effective amount of antibody, or antigen fragment thereof, wherein said antibody specifically binds to and inhibits insulin-like growth factor 1 receptor and is delivered by infusion.

### Enumerated Embodiments

Provided as embodiment 1 is a method of treating or reducing effects of scleroderma comprising administering to the subject an effective amount of an insulin-like growth factor-1 receptor (IGF-1R) inhibitor (e.g., an anti-IGF-1R antibody or an antigen binding fragment thereof or a small molecule IGF-1R inhibitor).

Embodiment 2. The method of Embodiment 1, wherein the scleroderma is localized scleroderma.

Embodiment 3. The method of Embodiment 2, wherein the localized scleroderma is morphea scleroderma or linear scleroderma.

Embodiment 4. The method of Embodiment 1, wherein the scleroderma is systemic scleroderma.

Embodiment 5. The method of Embodiment 4, wherein the systemic scleroderma is selected from the group consisting of limited cutaneous systemic scleroderma, systemic sclerosis sine scleroderma, and diffuse cutaneous systemic sclerosis.

Embodiment 6. The method of Embodiment 5, wherein the systemic scleroderma is diffuse cutaneous systemic sclerosis.

Embodiment 7. A method of treating interstitial lung disease (ILD) comprising administering to a subject in need thereof a therapeutically effective amount of an insulin-like growth factor-1 receptor (IGF-1R) inhibitor (e.g., an anti-IGF-1R antibody or an antigen binding fragment thereof or a small molecule IGF-1R inhibitor).

Embodiment 8. The method of Embodiment 7, wherein the ILD is idiopathic pulmonary fibrosis.

Embodiment 9. A method of reducing fibrosis and/or collagen production and/or accumulation in a subject with scleroderma or interstitial lung disease (ILD), comprising administering to said subject a therapeutically effective amount of an insulin-like growth factor-1 receptor (IGF-1R) inhibitor (e.g., an anti-IGF-1R antibody or an antigen binding fragment thereof or a small molecule IGF-1R inhibitor).

Embodiment 10. The method of Embodiment 9, wherein the subject has scleroderma.

Embodiment 11. The method of Embodiment 10, wherein the scleroderma is systemic scleroderma.

Embodiment 12. The method of Embodiment 11, wherein the systemic scleroderma is selected from the group consisting of limited cutaneous systemic scleroderma, systemic sclerosis sine scleroderma, and diffuse cutaneous systemic sclerosis.

Embodiment 13. The method of Embodiment 12, wherein the systemic scleroderma is diffuse cutaneous systemic sclerosis.

Embodiment 14. The method of Embodiment 9, wherein reducing fibrosis and collagen production and/or accumulation is measured by the skin elasticity.

Embodiment 15. The method of Embodiment 14, wherein reducing fibrosis and collagen production and/or accumulation is measured as a decrease in the subject's modified Rodnan total skin score (mRSS or mRTSS).

Embodiment 16. The method of any of Embodiments 9-15, wherein reducing fibrosis and collagen production and/or accumulation is measured as an increase in the subject's American College of Rheumatology-Composite Response Index in Systemic Sclerosis (ACR-CRISS) score.

Embodiment 17. The method of Embodiment 9, wherein the subject has ILD.

Embodiment 18. The method of Embodiment 17, wherein the ILD is idiopathic pulmonary fibrosis.

Embodiment 19. The method of any of Embodiments 9-13, 17, and 18, wherein reducing fibrosis and collagen production and/or accumulation is measured as an improvement in the subject's lung function.

Embodiment 20. The method of any of Embodiment 19, wherein reducing fibrosis and collagen production and/or accumulation is measured as an increase in the subject's walking distance under the 6-minute-walk test (6MWT).

Embodiment 21. The method of Embodiment 19, wherein the forced vital capacity (FVC) is improved by ≥5%.

Embodiment 22. The method of Embodiment 19, wherein the oxygen diffusing capacity (DLCO) is improved.

Embodiment 23. The method of any of embodiments 1-22, wherein the administration is by intradermal injection, subcutaneous injection, intravenous injection (including intravenous infusion), or by inhalation.

Embodiment 24: The method of any of embodiments 1-22, wherein the insulin-like growth factor-1 receptor (IGF 1R) inhibitor is administered by a topical formulation, which could include a lotion, cream, ointment, patch, or any other method to deliver the inhibitor to the subject through the skin.

Embodiment 25: The method of any of embodiments 1-22, wherein the insulin-like growth factor-1 receptor (IGF 1R) inhibitor is administered by an intradermal injection to the subject.

Embodiment 26: The method of any of embodiments 1-22, wherein the insulin-like growth factor-1 receptor (IGF 1R) inhibitor is administered by a subcutaneous injection to the subject.

Embodiment 27: The method of any of embodiments 1-22, wherein the insulin-like growth factor-1 receptor (IGF 1R) inhibitor is administered by an inhaler or nebulizer to the subject.

Embodiment 28: The method of any of embodiments 1-22, wherein the IGF 1R inhibitor is an anti-IGF-1R antibody or an antigen binding fragment thereof and is administered by infusion to the subject.

Embodiment 29: The method of any of Embodiments 1-23, wherein the IGF 1R inhibitor is an anti-IGF-1R antibody or an antigen binding fragment thereof and is administered at a dosage about 1 mg/kg to about 5 mg/kg antibody as a first dose.

Embodiment 30: The method of Embodiment 28, wherein the IGF 1R inhibitor is an anti-IGF-1R antibody or an antigen binding fragment thereof and is administered at a dosage about 5 mg/kg to about 10 mg/kg antibody as a first dose.

Embodiment 31: The method of Embodiment 29, wherein the IGF 1R inhibitor is an anti-IGF-1R antibody or an antigen binding fragment thereof and is administered at a dosage about 5 mg/kg to about 20 mg/kg antibody in subsequent doses.

Embodiment 32: The method of Embodiment 30, wherein the IGF 1R inhibitor is an anti-IGF-1R antibody or an antigen binding fragment thereof and is administered at a dosage about 10 mg/kg as a first dose; and about 20 mg/kg antibody in subsequent doses.

Embodiment 35: The method of Embodiment 31, wherein the subsequent doses are administered every three weeks for at least 21 weeks.

Embodiment 36: The method of any of Embodiments 28-35, wherein the antibody, or antigen binding fragment thereof, has a binding affinity (K_{D}) of 10⁻⁸M or less for the IGF-1R.

Embodiment 37: The method of Embodiment 36, wherein the antibody, or antigen binding fragment thereof, has a binding affinity (K_{D}) of 10⁻¹³ to 10⁻⁹ M for the IGF-1R.

Embodiment 38: The method of any of Embodiments 28-35, wherein the antibody, or antigen binding fragment thereof, has an IC50 values for the IGF1 and IGF2 to IGF-1R of no more than 2 nM.

Embodiment 39: The method of any of Embodiments 1-37, wherein the antibody, or an antigen binding fragment thereof, comprises a heavy chain comprising CDR1, CDR2, and CDR3 and a light chain comprising CDR1, CDR2 and CDR3, wherein the heavy chain CDR1, CDR2, and CDR3 amino acid sequences and light chain CDR1, CDR2, and CDR3 amino acid sequences are at least 90% identical to (i) the amino acid sequences of SEQ ID NOs: 85-90, respectively; or (ii) the amino acid sequences of SEQ ID NOs: 85, 93, 87, 88, 94, and 90, respectively.

Embodiment 40: The method of any of Embodiments 1-37, wherein the antibody, or an antigen binding fragment thereof, comprises: (i) heavy chain CDR1, CDR2, and CDR3 amino acid sequences and light chain CDR1, CDR2, and CDR3 amino acid sequences as set forth in SEQ ID NOs: 85-90, respectively; or (ii) heavy chain CDR1, CDR2, and CDR3 amino acid sequences and light chain CDR1, CDR2, and CDR3 amino acid sequences as set forth in SEQ ID NOs: 85, 93, 87, 88, 94, and 90, respectively.

Embodiment 41: The method of any of Embodiments 1-37, wherein the antibody, or an antigen binding fragment thereof, comprises: (i) a heavy chain variable region having at least 80% sequence identity to the amino acid sequence of SEQ ID NO: 91 and a light chain variable region having at least 80% sequence identity to the amino acid sequence of SEQ ID NO: 92; or (ii) a heavy chain variable region having at least 80% sequence identity to the amino acid sequence of SEQ ID NO: 95 and a light chain variable region having at least 80% sequence identity to the amino acid sequence of SEQ ID NO: 96.

Embodiment 42: The method of any of Embodiments 1-37, wherein the antibody, or an antigen binding fragment thereof, comprises: (i) a heavy chain variable region comprising the amino acid sequence of SEQ ID NO: 91 and a light chain variable region comprising the amino acid sequence of SEQ ID NO: 92; or (ii) a heavy chain variable region comprising the amino acid sequence of SEQ ID NO: 95 and a light chain variable region comprising the amino acid sequence of SEQ ID NO: 96.

Embodiment 43: The method of any of Embodiments 1-37, wherein the antibody is antibody 1 or antibody 2, or an antigen binding fragment thereof.

Embodiment 44: The method of any of Embodiments 1-37, wherein the antibody is Teprotumumab.

Embodiment 45: The method of any of any of Embodiments 1-44, wherein the antibody, or antigen binding fragment thereof, is a human antibody, a monoclonal antibody, a human monoclonal antibody, a purified antibody, a diabody, a single-chain antibody, a multispecific antibody, Fab, Fab′, F(ab′)2, Fv or scFv.

Embodiment 46: The method of any of Embodiments 1-45, wherein the antibody, or antigen binding fragment thereof, is administered in a pharmaceutical composition that additionally comprises a pharmaceutically acceptable diluent or carrier.

Embodiment 47: The method of any of Embodiments 1-46, additionally comprising administering, as part of the pharmaceutical composition or separately, one or more other pharmaceutically active compounds for the treatment of morphea or linear scleroderma.

Embodiment 48: The method of any of Embodiments 1-46, wherein the additional administration, as part of the pharmaceutical composition or separately, a compound chosen from: a corticosteroid; Rituximab or another anti-CD20 antibody; Tocilizumab of another anti-IL-6 antibody; or methotrexate is used in the treatment of scleroderma.

Embodiment 49: The method of any of Embodiments 1-46, wherein the treatment is efficacious for a least 4 weeks beyond the last administered dose.

Embodiment 50: The method of Embodiment 49, wherein the treatment is efficacious for a least 6 weeks beyond the last administered dose.

Embodiment 51: The method of Embodiment 50, wherein the treatment is efficacious for a least 8 weeks beyond the last administered dose.

Embodiment 52: The method of Embodiment 51, wherein the treatment is efficacious for a least 20 weeks beyond the last administered dose.

Embodiment 53. The method of any of Embodiments 1-52 wherein the IGF-1R inhibitor is an antibody or small molecule.

Embodiment 54. The method of Embodiment 53 wherein said IGF-1R inhibitor is chosen from ganitumab, figitumumab, MEDI-573, cixutumumab, dalotuzumab, robatumumab, AVE1642, BIIB022, xentuzumab, istiratumab, linsitinib, picropodophyllin, BMS-754807, BMS-536924, BMS-554417, GSK1838705A, GSK1904529A, NVP-AEW541, NVP-ADW742, GTx-134, AG1024, KW-2450, PL-2258, NVP-AEW541, NSM-18, AZD3463, AZD9362, BI885578, BI893923, TT-100, XL-228, and A-928605.

Embodiment 55. The method of Embodiment 53 wherein said IGF-1R inhibitor is an antibody.

Embodiment 56. The method of Embodiment 54 wherein said IGF-1R inhibitor is a human, chimeric human, or humanized monoclonal antibody suitable for human therapy.

Embodiment 57. The method of Embodiment 56 wherein the antibody is administered intravenously (IV) or subcutaneously (SC).

Embodiment 58. The method of Embodiment 56 wherein the antibody is administered IV.

Embodiment 59. The method of Embodiment 57 wherein said antibody is chosen from ganitumab, figitumumab, MEDI-573, cixutumumab, dalotuzumab, robatumumab, AVE1642, BIIB022, xentuzumab, and istiratumab.

Embodiment 60. The method of Embodiment 59 wherein the antibody is ganitumab.

Embodiment 61. The method of Embodiment 60 wherein the ganitumab is dosed at:
a) 1-60 mg/kg or 75-4500 mg IV every 3 weeks; or
b) 0.6-40 mg/kg or 45-3000 mg IV every 2 weeks; or
c) 0.3-20 mg/kg; or 22-1500 mg IV weekly.

Embodiment 62. The method of Embodiment 59 wherein the antibody is figitumumab.

Embodiment 63. The method of Embodiment 62 wherein the figitumumab is dosed at:
a) 1-60 mg/kg or 75-4500 mg IV every 3 weeks; or
b) 0.6-40 mg/kg or 45-3000 mg IV every 2 weeks; or
c) 0.3-20 mg/kg or 22-1500 mg IV weekly.

Embodiment 64. The method of Embodiment 59 wherein the antibody is cixutumumab.

Embodiment 65. The method of Embodiment 64 wherein the cixutumumab is dosed at:
a) 1-45 mg/kg or 75-3400 mg IV every 3 weeks; or
b) 0.6-30 mg/kg or 45-2300 mg IV every 2 weeks; or
c) 0.3-15 mg/kg Or 22-1200 mg IV weekly.

Embodiment 66. The method of Embodiment 59 wherein the antibody is dalotuzumab.

Embodiment 67. The method of Embodiment 66 wherein the dalotuzumab is dosed at:
a) 1-90 mg/kg or 75-6800 mg IV every 3 weeks; or
b) 0.6-60 mg/kg or 45-4500 mg IV every 2 weeks; or
c) 0.3-30 mg/kg or 22-2300 mg IV weekly.

Embodiment 68. The method of Embodiment 59 wherein the antibody is robatumumab.

Embodiment 69. The method of Embodiment 68 wherein the robatumumab is dosed at:
a) 1-75 mg/kg or 75-5700 mg IV every 3 weeks; or
b) 0.6-50 mg/kg or 45-3800 mg IV every 2 weeks; or
c) 0.3-25 mg/kg or 22-1900 mg IV weekly.

Embodiment 70. The method of Embodiment 59 wherein the antibody is xentuzumab.

Embodiment 71. The method of Embodiment 70 wherein the xentuzumab is dosed at:
a) 1-112 mg/kg or 75-8400 mg IV every 3 weeks; or
b) 0.6-75 mg/kg or 45-5700 mg IV every 2 weeks; or
c) 0.3-38 mg/kg or 22-2900 mg IV weekly.

Embodiment 72. The method of Embodiment 59 wherein the antibody is istiratumab.

Embodiment 73. The method of Embodiment 72 wherein the istiratumab is dosed at:
a) 1-112 mg/kg or 75-8400 mg IV every 3 weeks; or
b) 0.6-75 mg/kg or 45-5700 mg IV every 2 weeks; or
c) 0.3-38 mg/kg or 22-2900 mg IV weekly.

Embodiment 74. The method of Embodiment 59 wherein the antibody is AVE1642.

Embodiment 75. The method of Embodiment 74 wherein the AVE1642 is dosed at:
a) 1-60 mg/kg or 75-4500 mg IV every 3 weeks; or
b) 0.6-40 mg/kg or 45-3000 mg IV every 2 weeks; or
c) 0.3-20 mg/kg or 22-1500 mg IV weekly.

Embodiment 76. The method of Embodiment 59 wherein the antibody is BIIB022.

Embodiment 77. The method of Embodiment 76 wherein the BIIB022 is dosed at:
a) 1-75 mg/kg or 75-5700 mg IV every 3 weeks; or
b) 0.6-50 mg/kg; or 45-3800 mg IV every 2 weeks; or
c) 0.3-25 mg/kg or 22-1900 mg IV weekly.

Embodiment 78. The method of Embodiment 59 wherein said IGF-1R inhibitor antibody comprises at least one heavy chain and at least one light chain selected from the selected from the group consisting of:
a) a heavy chain comprising the amino acid sequence of SEQ ID NO:7 and a light chain comprising the amino acid sequence SEQ ID NO:8;
b) a heavy chain comprising the amino acid sequence of SEQ ID NO:15 and a light chain comprising the amino acid sequence SEQ ID NO:16;
c) a heavy chain comprising the amino acid sequence of SEQ ID NO:23 and a light chain comprising the amino acid sequence SEQ ID NO:24;
d) a heavy chain comprising the amino acid sequence of SEQ ID NO:31 and a light chain comprising the amino acid sequence SEQ ID NO:32;
e) a heavy chain comprising the amino acid sequence of SEQ ID NO:39 and a light chain comprising the amino acid sequence SEQ ID NO:40;
f) a heavy chain comprising the amino acid sequence of SEQ ID NO:47 and a light chain comprising the amino acid sequence SEQ ID NO:48;
g) a heavy chain comprising the amino acid sequence of SEQ ID NO:55 and a light chain comprising the amino acid sequence SEQ ID NO:56;
h) a heavy chain comprising the amino acid sequence of SEQ ID NO:63 and a light chain comprising the amino acid sequence SEQ ID NO:64;
i) a heavy chain comprising the amino acid sequence of SEQ ID NO:65 and a light chain comprising the amino acid sequence SEQ ID NO:66; and
j) a heavy chain comprising the amino acid sequence of SEQ ID NO:73 and a light chain comprising the amino acid sequence SEQ ID NO:74.
k) a heavy chain comprising the amino acid sequence of SEQ ID NO:31 and a light chain comprising the amino acid sequence SEQ ID NO:81;
l) a heavy chain comprising the amino acid sequence of SEQ ID NO:31 and a light chain comprising the amino acid sequence SEQ ID NO:82;
m) a heavy chain comprising the amino acid sequence of SEQ ID NO:31 and a light chain comprising the amino acid sequence SEQ ID NO:83;
n) a heavy chain comprising the amino acid sequence of SEQ ID NO:31 and a light chain comprising the amino acid sequence SEQ ID NO:84;
o) a heavy chain comprising the amino acid sequence of SEQ ID NO:79 and a light chain comprising the amino acid sequence SEQ ID NO:32;
p) a heavy chain comprising the amino acid sequence of SEQ ID NO:79 and a light chain comprising the amino acid sequence SEQ ID NO:81;
q) a heavy chain comprising the amino acid sequence of SEQ ID NO:79 and a light chain comprising the amino acid sequence SEQ ID NO:82;
r) a heavy chain comprising the amino acid sequence of SEQ ID NO:79 and a light chain comprising the amino acid sequence SEQ ID NO:83;
s) a heavy chain comprising the amino acid sequence of SEQ ID NO:79 and a light chain comprising the amino acid sequence SEQ ID NO:84

Embodiment 79. The method of Embodiment 36 wherein said IGF-1R inhibitor is a small molecule.

Embodiment 80. The method of Embodiment 61 wherein said IGF-1R inhibitor is dosed orally.

Embodiment 81. The method of Embodiment 63 wherein said IGF-1R inhibitor is chosen from linsitinib, picropodophyllin, BMS-754807, BMS-536924, BMS-554417, GSK1838705A, GSK1904529A, NVP-AEW541, NVP-ADW742, GTx-134, AG1024, KW-2450, PL-2258, NVP-AEW541, NSM-18, AZD3463, AZD9362, BI885578, BI893923, TT-100, XL-228, and A-928605.

Embodiment 82. The method of Embodiment 81 wherein the IGF-1R inhibitor is linsitinib.

Embodiment 83. The method of Embodiment 65 wherein the linsitinib is dosed at:
a) 10-750 mg orally once daily continuous dosing or 10-1500 mg/day for once daily intermittent dosing (for up to 7 days of every 14 days); or
b) 6-500 mg orally twice daily continuous dosing or 6-1000 mg for twice daily intermittent dosing (for up to 7 days of every 14 days); or
c) 3-250 mg orally three-times daily continuous dosing or 3-500 mg for three-times daily intermittent dosing (for up to 7 days of every 14 days).

Embodiment 84. The method of Embodiment 81 wherein the IGF-1R inhibitor is picropodophyllin.

Embodiment 85. The method of Embodiment 67 wherein the picropodophyllin is dosed:
a) orally once daily at 20-2000 mg; or
b) orally twice daily at 13-1400 mg; or
c) orally three times daily at 6-700 mg.

Embodiment 86. The method of Embodiment 81 wherein the IGF-1R inhibitor is BMS-754807.

Embodiment 87. The method of Embodiment 69 wherein the BMS-754807 is dosed:
a) once daily at 5-600 mg orally; or
b) twice daily at 3-400 mg orally; or
c) three times daily at 1-200 mg.

Embodiment 88. The method of Embodiment 81 wherein the IGF-1R inhibitor is BMS-536924.

Embodiment 89. The method of Embodiment 81 wherein the IGF-1R inhibitor is BMS-554417.

Embodiment 90. The method of Embodiment 81 wherein the IGF-1R inhibitor is GSK1838705A.

Embodiment 91. The method of Embodiment 81 wherein the IGF-1R inhibitor is GSK1904529A.

Embodiment 92. The method of Embodiment 81 wherein the IGF-1R inhibitor is NVP-AEW541.

Embodiment 93. The method of Embodiment 81 wherein the IGF-1R inhibitor is NVP-ADW742.

Embodiment 94. The method of Embodiment 81 wherein the IGF-1R inhibitor is GTx-134.

Embodiment 95. The method of Embodiment 81 wherein the IGF-1R inhibitor is AG1024.

Embodiment 96. The method of Embodiment 81 wherein the IGF-1R inhibitor is PL-2258.

Embodiment 97. The method of Embodiment 81 wherein the IGF-1R inhibitor is NVP-AEW541.

Embodiment 98. The method of Embodiment 81 wherein the IGF-1R inhibitor is NSM-18.

Embodiment 99. The method of Embodiment 81 wherein the IGF-1R inhibitor is AZD3463.

Embodiment 100. The method of Embodiment 81 wherein the IGF-1R inhibitor is AZD9362.

Embodiment 101. The method of Embodiment 81 wherein the IGF-1R inhibitor is BI885578.

Embodiment 102. The method of Embodiment 81 wherein the IGF-1R inhibitor is BI893923.

Embodiment 103. The method of Embodiment 81 wherein the IGF-1R inhibitor is TT-100.

Embodiment 104. The method of Embodiment 81 wherein the IGF-1R inhibitor is XL-228.

Embodiment 105. The method of Embodiment 81 wherein the IGF-1R inhibitor is A-928605.

Embodiment 106. The method of any of Embodiments 88-105 wherein the IGF-1R inhibitor is dosed:
a) once daily at 1-2000 mg orally; or
b) twice daily at 0.6-1400 mg orally; or
c) three times daily at 0.3-700 mg orally.

Embodiment 107. The method of Embodiment 81 wherein the IGF-1R inhibitor is KW-2450.

Embodiment 108. The method of Embodiment 107 wherein the KW-2450 is dosed:
a) once daily at 1-100 mg orally; or
b) twice daily at 0.6-70 mg orally; or
c) three times daily at 0.3-30 mg orally.

Embodiment 109. The method of any of embodiments 1-78, wherein the anti-insulin-like growth factor-1 receptor (IGF-1R) antibody or an antigen binding fragment thereof comprises a modification in the Fc region to extend the half-life.

Provided as Embodiment 110 is a method to reduce the mRSS score or improve the results of a skin biopsy test in a subject with scleroderma, comprising administering to the subject an effective amount of an antibody, or an antigen binding fragment thereof, wherein the antibody specifically binds to and inhibits insulin-like growth factor 1 receptor (IGF-1R).

Embodiment 111: The method of Embodiment 110, wherein the mRSS score is improved by at least 3 points or there is improvement in a skin biopsy test in a subject with scleroderma.

Embodiment 112: The method of Embodiment 110, wherein the mRSS score is improved by at least 5 points or there is improvement in a skin biopsy test in a subject with scleroderma.

Embodiment 113: The method of Embodiment 110, wherein the mRSS score is improved by at least 7 points or there is improvement in a skin biopsy test in a subject with scleroderma.

Provided as Embodiment 114 is a method for increasing the ACR-CRISS score of a subject, comprising administering to the subject an effective amount of an antibody, or an antigen binding fragment thereof, wherein the antibody specifically binds to and inhibits insulin-like growth factor 1 receptor (IGF-1R).

Embodiment 115: The method of embodiment 114, wherein the ACR-CRISS score is improved by at least 0.10 points in a subject with systemic scleroderma.

Embodiment 116: The method of embodiment 114, wherein the ACR-CRISS score is improved by at least 0.15 points in a subject with systemic scleroderma.

Embodiment 117: The method of embodiment 114, wherein the ACR-CRISS score is improved by at least 0.20 points in a subject with systemic scleroderma.

Provided as Embodiment 118 is a method for increasing the distance walked in the 6-minute-walk test of a subject, comprising administering to the subject an effective amount of an antibody, or an antigen binding fragment thereof, wherein the antibody specifically binds to and inhibits insulin-like growth factor 1 receptor (IGF-1R).

Embodiment 119: The method of embodiment 118, wherein the 6MWT distance is improved by a least 5 meters.

Embodiment 120: The method of embodiment 118, wherein the 6MWT distance is improved by a least 10 meters.

Embodiment 121: The method of embodiment 118, wherein the 6MWT distance is improved by a least 25 meters.

Embodiment 122: The method of embodiment 118, wherein the 6MWT distance is improved by a least 40 meters.

Also provided as Embodiments 123-210 are embodiments equivalent to embodiments 23-109, depending instead from any of the methods of embodiments 110-122.

Additional embodiments are disclosed throughout the specification.

### Tests

Measurement of the degree of hardness of the skin by the modified Rodnan total skin score (mRSS or mRTSS). This method uses palpation of the skin in 17 areas of the body and providing a score between zero and three for each area. Zero refers to no thinkening, one is mild thinkening, two is moderate thickening, and three is severe thickening of the skin. A typical mRSS score for a scleroderma patient falls between 16-27.

American College of Rheumatology-Composite Response Index in Systemic Sclerosis (ACR-CRISS or CRISS) score is calculated from weighted changes from baseline in five core outcome measures commonly used to evaluate treatment effect in trials for systemic scleroderma: mRSS, Health Assessment Questionnaire-Disability Index (HAQ-DI), forced vital capacity (FVC) percent predicted, and patient and physician global assessments of health related to systemic scleroderma.

### Definitions

The following terms shall be understood to have the meanings ascribed herein.

The term “about,” as used herein in relation to a numerical value x means x±10%.

The term “and/or” when used in a list of two or more items, means that any one of the listed items can be employed by itself or in combination with any one or more of the listed items. For example, the expression “A and/or B” is intended to mean either or both of A and B, i.e., A alone, B alone or A and B in combination. The expression “A, B and/or C” is intended to mean A alone, B alone, C alone, A and B in combination, A and C in combination, B and C in combination or A, B, and C in combination.

As used herein, the term “antibody” encompasses the various forms of antibodies including but not being limited to whole antibodies, monoclonal antibodies, antibody fragments, human antibodies, humanized antibodies, chimeric antibodies and genetically engineered antibodies as long as the characteristic properties such as specificity and IGF-IR inhibitory are retained.

As used herein, the terms “antigen binding fragment,” “fragment,” and “antibody fragment” are used interchangeably to refer to any fragment that comprises a portion of a full length antibody, generally at least the antigen binding portion or the variable region thereof. Examples of antibody fragments include, but are not limited to, diabodies, single-chain antibody molecules, multispecific antibodies, Fab, Fab′, F(ab′)₂, Fv or scFv. Further, the term “antibody” as used herein includes both antibodies and antigen binding fragments thereof. In addition, antibody fragments comprise single chain polypeptides having the characteristics of a VH chain, namely being able to assemble together with a VL chain or of a VL chain binding to IGF-IR, namely being able to assemble together with a VH chain to a functional antigen binding pocket and thereby providing the property of inhibiting the binding of IGF-I and IGF-II to IGF-IR.

The terms “binding to IGF-IR” or “specific binding to IGF-IR” are used interchangeably herein and mean the binding of the antibody to IGF-IR in an in vitro assay, preferably in a binding assay in which the antibody is bound to a surface and binding of IGF-IR is measured by Surface Plasmon Resonance (SPR). Binding means a binding affinity (K_{D}) of 10⁻⁸ M or less, preferably 10⁻¹³ to 10⁻⁹ M. Binding to IGF-IR can be investigated by a BIAcore assay (Pharmacia Biosensor AB, Uppsala, Sweden). The affinity of the binding is defined by the terms ka (rate constant for the association of the antibody from the antibody/antigen complex), kd (dissociation constant), and K_{D} (kd/ka). The antibodies used in the methods disclosed herein show a K_{D} of about 10⁻⁹ M or less.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The terms “complementarity determining region,” “CDR,” “hypervariable region,” or “antigen-binding portion of an antibody” are used interchangeably herein and refer to the amino acid residues of an antibody which are responsible for antigen-binding. The hypervariable region comprises amino acid residues from the complementarity determining regions or CDRs. “Framework” or “FR” regions are those variable domain regions other than the hypervariable region residues as herein defined. Therefore, the light and heavy chains of an antibody comprise from N- to C-terminus the domains FR1, CDR1, FR2, CDR2, FR3, CDR3, and FR4. Especially, CDR3 of the heavy chain is the region which contributes most to antigen binding. CDR and FR regions are determined according to the standard definition of Kabat et al., Sequences of Proteins of Immunological Interest, 5th Ed. Public Health Service, National Institutes of Health, Bethesda, Md. (1991)) and/or those residues from a “hypervariable loop.”

The term “comprising” encompasses “including” as well as “consisting” e.g., a composition “comprising” X may consist exclusively of X or may include something additional e.g., X+Y.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “human antibody” as used herein, is intended to include antibodies having variable and constant regions derived from human germline immunoglobulin sequences. The term “humanized antibody” as used herein refers to antibodies in which the framework or “complementarity determining regions” (CDR) have been modified to comprise the CDR of an immunoglobulin of different specificity as compared to that of the parent immunoglobulin. In a preferred embodiment, a murine CDR is grafted into the framework region of a human antibody to prepare the “humanized antibody.”

The term “IGF-1R inhibitor” as used herein means a compound (e.g., small molecule or antibody, including antigen-binding fragments thereof) which specifically binds to and inhibits insulin-like growth factor 1 receptor (IGF-1R).

The term “inhibiting the binding of IGF-I and IGF-II to IGF-IR” as used herein refers to inhibiting the binding of I¹²⁵-labeled IGF-I or IGF-II to IGF-IR presented on the surface of cells in an in vitro assay. Inhibiting means an IC₅₀ value of 2 nM or lower.

The terms “monoclonal antibody” or “monoclonal antibody composition,” as used herein refer to a preparation of antibody molecules of a single amino acid composition. Accordingly, the term “human monoclonal antibody” refers to antibodies displaying a single binding specificity which have variable and constant regions derived from human germline immunoglobulin sequences. In one embodiment, the human monoclonal antibodies are produced by a hybridoma which includes a B cell obtained from a transgenic non-human animal, e.g., a transgenic mouse, having a genome comprising a human heavy chain transgene and a light human chain transgene fused to an immortalized cell.

The term “recombinant human antibody,” as used herein, is intended to include all human antibodies that are prepared, expressed, created or isolated by recombinant means, such as antibodies isolated from a host cell such as an SP2-0, NS0 or CHO cell or from an animal (e.g., a mouse) that is transgenic for human immunoglobulin genes or antibodies expressed using a recombinant expression vector transfected into a host cell. Such recombinant human antibodies have variable and constant regions derived from human germline immunoglobulin sequences in a rearranged form.

The term “Scleroderma” is the chronic hardening and contraction of the skin and connective tissue, either locally or throughout the body. It can refer to all forms of the disease or a single form to the disease. These forms include morphea, linear, limited systemic, and diffuse systemic.

The terms “subject” and “patient” are used interchangeably herein to mean all mammals including humans Examples of subjects include, but are not limited to, humans, monkeys, dogs, cats, horses, cows, goats, sheep, pigs, and rabbits. In one embodiment, the subject or patient is a human. The terms “affected with a disease or disorder,” “afflicted with a disease or disorder,” or “having a disease or disorder” are used interchangeably herein and refer to a subject or patient with any disease, disorder, syndrome or condition. No increased or decreased level of severity of the disorder is implied by the use of one the terms as compared to the other.

The word “substantially” does not exclude “completely” e.g., a composition which is “substantially free” from Y may be completely free from Y. Where necessary, the word “substantially” may be omitted.

The term “teprotumumab,” also known as RV-001 and R-1507, is a human monoclonal antibody that binds to insulin-like growth factor-1 receptor (IGF-1R). It has CAS number 1036734-93-6 and comprises a SEQ ID NO.S 1-8 disclosed herein (see, e.g., table 17). It comprises and may be referred to in the alternative throughout this disclosure as “Antibody 1.”

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

The phrase “therapeutically effective” is intended to qualify the amount of active ingredients used in the treatment of a disease or disorder or on the effecting of a clinical endpoint.

The terms “treating,” “treatment,” and the like, as used herein, mean ameliorating a disease, so as to reduce, ameliorate, or eliminate its cause, its progression, its severity, or one or more of its symptoms, or otherwise beneficially alter the disease in a subject. Reference to “treating,” or “treatment” of a patient is intended to include prophylaxis. Treatment may also be preemptive in nature, i.e., it may include prevention of disease in a subject exposed to or at risk for the disease. Prevention of a disease may involve complete protection from disease, for example as in the case of prevention of infection with a pathogen, or may involve prevention of disease progression, for example from prediabetes to diabetes. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

The domains of variable human light and heavy chains have the same general structure and each domain comprises four framework (FR) regions whose sequences are widely conserved, connected by three “hypervariable regions” (or complementarity determining regions, CDRs). The framework regions adopt a β-sheet conformation and the CDRs may form loops connecting the β-sheet structure. The CDRs in each chain are held in their three-dimensional structure by the framework regions and form together with the CDRs from the other chain the antigen binding site. The antibody heavy and light chain CDR3 regions play an important role in the binding specificity/affinity of antibodies.

The antibodies, or antigen binding fragments thereof, used in the methods disclosed herein inhibit the binding of IGF-I and IGF-II to IGF-IR. The inhibition is measured as IC₅₀ in an assay for binding of IGF-I/IGF-II to IGF-IR on cells. Such an assay is known to one of skill in the art and is described, for example, U.S. Pat. No. 7,579,157, which is incorporated herein in its entirety. The IC₅₀ values of the antibodies used in the methods disclosed herein for the binding of IGF-I and IGF-II to IGF-IR are no more than 2 nM. IC₅₀ values are measured as average or median values of at least three independent measurements. Single IC₅₀ values may be out of the scope.

When introducing elements of the present disclosure or the preferred embodiment(s) thereof, the articles “a,” “an,” “the” and “said” are intended to mean that there are one or more of the elements. The terms “comprising,” “including” and “having” are intended to be inclusive and mean that there may be additional elements other than the listed elements.

When ranges of values are disclosed, and the notation “from n1 . . . to n2” or “between n1 . . . and n2” is used, where n1 and n2 are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

### Antibodies

The sequences of the heavy chains and light chains of examples of antibodies that may be used in the methods disclosed herein, each comprising three CDRs on the heavy chain and three CDRs on the light chain are provided below. The sequences of the CDRs, heavy chains, light chains as well as the sequences of the nucleic acid molecules encoding the CDRs, heavy chains and light chains of the antibodies are disclosed in the sequence listing. The CDRs of the antibody heavy chains are referred to as CDRH1 (or HCDR1), CDRH2 (or HCDR2) and CDRH3 (or HCDR3), respectively. Similarly, the CDRs of the antibody light chains are referred to as CDRL1 (or LCDR1), CDRL2 (or LCDR2) and CDRL3 (or LCDR3), respectively. Table 2 provides the SEQ ID numbers for the amino acid sequences of the six CDRs of the heavy and light chains, respectively, of the antibodies that may be used in the methods disclosed herein.

TABLE 2
SEQ ID Number for CDR Polypeptides of Antibodies Disclosed Herein.

| | SEQ ID NOs. for CDR Polypeptides | | | | | |
|---|---|---|---|---|---|---|
| | CDRH1 | CDRH2 | CDRH3 | CDRL1 | CDRL2 | CDRL3 |
| Antibody 1 | 85 | 86 | 87 | 88 | 89 | 90 |
| Antibody 2 | 85 | 93 | 87 | 88 | 94 | 90 |

In one embodiment, an antibody or antibody fragment useful in the methods disclosed herein comprises at least one CDR with a sequence that has at least 95% sequence identity to any one of SEQ ID NOs: 85-90, 93, or 94 and specifically inhibits (or blocks) Insulin Like Growth Factor-I Receptor (IGF-IR).

In another embodiment, the antibody or antigen binding fragment that can be used in the methods comprising a heavy chain comprises one or more (i.e. one, two or all three) heavy chain CDRs from antibody 1 or antibody 2 and specifically inhibits or blocks IGF-IR.

In yet another embodiment, the antibody or antigen binding fragment useful in the methods disclosed herein comprises a heavy chain CDR1 with the amino acid sequence of SEQ ID NO: 85; a heavy chain CDR2 with the amino acid sequence of SEQ ID NO: 86, or SEQ ID NO:93; and a heavy chain CDR3 with the amino acid sequence of SEQ ID NO: 87. In certain embodiments, the antibody or antibody fragment comprises a heavy chain comprising the amino acid sequence of (i) SEQ ID NO: 85 for CDRH1, SEQ ID NO: 86 for CDRH2 and SEQ ID NO: 87 for CDRH3; or (ii) SEQ ID NO: 85 for CDRH1, SEQ ID NO: 93 for CDRH2, and SEQ ID NO: 87 for CDRH3 and specifically inhibits IGF-IR.

In another embodiment, the antibody or antigen binding fragment that can be used in the methods disclosed herein comprising a light chain comprising one or more (i.e. one, two or all three) light chain CDRs from antibody 1 or antibody 2 and specifically inhibits IGF-IR.

In yet another embodiment, an antibody or antibody fragment useful in the methods disclosed herein comprises a light chain CDR1 with the amino acid sequence of SEQ ID NO: 88; a light chain CDR2 with the amino acid sequence of SEQ ID NO: 89, or SEQ ID NO: 94; and a light chain CDR3 with the amino acid sequence of SEQ ID NO: 90. In certain embodiments, the antibody or antibody fragment comprises a light chain comprising the amino acid sequence of (i) SEQ ID NO: 88 for CDRL1, SEQ ID NO: 89 for CDRL2, and SEQ ID NO: 90 for CDRL3; or (ii) SEQ ID NO: 88 for CDRL1, SEQ ID NO: 94 for CDRL2, and SEQ ID NO: 90 for CDRL3.

In one embodiment, the antibody, or antigen binding fragment thereof, comprises all of the CDRs of antibody 1 as listed in Table 2, and specifically inhibits (or blocks) Insulin Like Growth Factor-I Receptor (IGF-IR). In another embodiment, an antibody, or antigen binding fragment thereof, comprises all of the CDRs of antibody 2 as listed in Table 2, and specifically inhibits (or blocks) IGF-IR.

The SEQ ID numbers for the amino acid sequence for the heavy chain variable region (VH) and the light chain variable region (VL) of antibodies useful in the methods disclosed herein are listed in Table 3.

TABLE 3
SEQ ID Numbers for V_{H} and V_{L} amino acid for
Antibodies Disclosed Herein.

| | V_{H} amino acid | V_{L} amino acid |
|---|---|---|
| Antibody 1 | 91 | 92 |
| Antibody 2 | 95 | 96 |

In one embodiment, the antibody or antigen binding fragment that can be used in the methods disclosed herein comprises a heavy chain variable region having an amino acid sequence that is about 70%, 75%, 80%, 85%, 90%, 95%, 97%, 98%, 99% or 100% identical to the sequence recited in SEQ ID NOs: 7 or 11 wherein the antibody specifically inhibits IGF-IR.

In another embodiment, the antibody or antigen binding fragment that can be used in the methods disclosed herein specifically inhibits IGF-IR and comprises a heavy chain variable region comprising the amino acid sequence of SEQ ID NO: 91 and a light chain variable region comprising the amino acid sequence of SEQ ID NO: 92; or a heavy chain variable region comprising the amino acid sequence of SEQ ID NO: 95 and a light chain variable region comprising the amino acid sequence of SEQ ID NO: 96.

Examples of antibodies useful in the methods disclosed herein include, but are not limited to, antibody 1, and antibody 2. In some embodiments, antibody 1 is teprotumumab.

Variant antibodies are also included within the scope of the disclosure. Thus, variants of the sequences recited in the application are also included within the scope of the disclosure. Such variants include natural variants generated by somatic mutation in vivo during the immune response or in vitro upon culture of immortalized B cell clones. Alternatively, variants may arise due to the degeneracy of the genetic code or may be produced due to errors in transcription or translation.

Further variants of the antibody sequences having improved affinity and/or potency may be obtained using methods known in the art and are included within the scope of the disclosure. For example, amino acid substitutions may be used to obtain antibodies with further improved affinity. Alternatively, codon optimization of the nucleotide sequence may be used to improve the efficiency of translation in expression systems for the production of the antibody. Further, polynucleotides comprising a sequence optimized for antibody specificity or neutralizing activity by the application of a directed evolution method to any of the nucleic acid sequences disclosed herein are also within the scope of the disclosure.

In one embodiment variant antibody sequences may share 70% or more (i.e. 75%, 80%, 85%, 90%, 95%, 97%, 98%, 99% or more) amino acid sequence identity with the sequences recited in the application. In some embodiments such sequence identity is calculated with regard to the full length of the reference sequence (i.e. the sequence recited in the application). In some further embodiments, percentage identity, as referred to herein, is as determined using BLAST version 2.1.3 using the default parameters specified by the NCBI (the National Center for Biotechnology Information; http://www.ncbi.nlm nih.gov/) [Blosum 62 matrix; gap open penalty=11 and gap extension penalty=1].

Antibodies used with the methods disclosed herein can be coupled to a drug for delivery to a treatment site or coupled to a detectable label to facilitate imaging of a site comprising cells of interest. Methods for coupling antibodies to drugs and detectable labels are well known in the art, as are methods for imaging using detectable labels. Labeled antibodies may be employed in a wide variety of assays, employing a wide variety of labels. Detection of the formation of an antibody-antigen complex between an antibody and an epitope of interest can be facilitated by attaching a detectable substance to the antibody and detecting the antibody-antigen complex by suitable detection means known to one of skill in the art.

Antibodies, or antigen binding fragments thereof, used with the methods disclosed herein can be of any isotype (e.g., IgA, IgG, IgM i.e. an α, γ or μ heavy chain). In one embodiment the antibody is IgG. Within the IgG isotype, antibodies may be IgG1, IgG2, IgG3 or IgG4 subclass. The antibodies may have a κ or a λ light chain.

The antibodies, or an antigen binding fragments thereof, used with the methods disclosed herein can be administered by any route known to one of skill in the art. Non-exhaustive examples of routes that can be used are provided below.

### Antibody Fc Variants and Half-Life

In immunoglobulins, such as IgG, a site in the Fc region of the heavy chain mediates interaction with the neonatal receptor (FcRn). Binding to FcRn recycles endocytosed antibody from the endosome back to the bloodstream and plays a key role in antibody transport. This process, coupled with preclusion of kidney filtration due to the large size of the full-length molecule, results in favorable antibody serum half-lives ranging from one to three weeks in vivo. Thus, the fidelity of this region on Fc is important for the clinical properties of antibodies.

Other properties of the antibody may determine its clearance rate (e.g. stability and half-life) in vivo. In addition to antibody binding to the FcRn receptor, other factors that contribute to clearance and half-life are serum aggregation, enzymatic degradation in the serum, inherent immunogenicity of the antibody leading to clearing by the immune system, antigen-mediated uptake, FcR (non-FcRn) mediated uptake and non-serum distribution (e.g. in different tissue compartments).

Accordingly, one means by which the pharmacokinetics (PK) and pharmacodynamics (PD) of a therapeutic antibody can by changed is by increasing the serum half-life of the antibody by altering the heavy constant domains within the Fc. In addition, due to the methodologies outlined herein, the possibility of immunogenicity resulting from the FcRn variants is significantly reduced by importing variants from different IgG isotypes such that serum half-life is increased without introducing significant immunogenicity.

The substitutions in the Fc domains are chosen such that the resultant proteins show improved serum half-life in vivo as compared to the wild type protein. In order to increase the retention of the Fc proteins in vivo, the increase in binding affinity must be at around pH 6 while maintaining lower affinity at around pH 7.4. Without being limited to theory, Fc regions are believed to have longer half-lives in vivo because binding to FcRn at pH 6 in an endosome sequesters the Fc. The endosomal compartment then recycles the Fc to the cell surface. Once the compartment opens to the extracellular space, the higher pH (˜7.4) induces the release of Fc back into the blood. The increased affinity of Fc for FcRn at pH 7.4 is thought to forbid the release of the Fc back into the blood. As a result, Fc mutations that increase Fc's half-life in vivo generally increase FcRn binding at the lower pH while still allowing release of Fc at higher pH. The amino acid histidine changes its charge state in the pH range of 6.0 to 7.4. Therefore, it is not surprising to find histidine residues at important positions in the Fc/FcRn complex.

In some embodiments, the increase in FcRn binding over wild type specifically at lower pH (˜6.0) facilitates Fc/FcRn binding in the endosome. In some embodiments, Fc variants with altered FcRn binding can have altered binding to another class of Fc receptors, the FcγR's (FcgammaR's) as differential binding to FcγR5, particularly increased binding to FcγRIIIb and decreased binding to FcγRIIb, has been shown to result in increased efficacy.

In some embodiments, importation of substitutions at particular positions from one IgG isotype into another can be achieved, thus reducing or eliminating the possibility of unwanted immunogenicity being introduced into the variants. That is, IgG1 is a common isotype for therapeutic antibodies for a variety of reasons, including high effector function. IgG2 residues at particular positions can be introduced into the IgG1 backbone to result in a protein that exhibits longer serum half-life.

In some embodiments, non-isotypic amino acid changes are made, to improve binding to FcRn and/or to increase in vivo serum half-life, and/or to allow accommodations in structure for stability, etc.

As will be appreciated by those in the art and described below, a number of factors contribute to the in vivo clearance, and thus the half-life, of antibodies in serum. One factor involves the antigen to which the antibody binds; that is, antibodies with identical constant regions but different variable regions (e.g., Fv domains), may have different half-lives due to differential ligand binding effects. However, the present disclosure demonstrates that while the absolute half-life of two different antibodies may differ due to these antigen specificity effects, the FcRn variants described herein, can transfer to different ligands to give the same trends of increasing half-life. That is, in general, the relative “order” of the FcRn binding/half-life increases will track to antibodies with the same variants of antibodies with different Fvs as is discussed herein.

Fc variants within a therapeutic antibody are made by introducing amino acid mutations into the parent molecule. “Mutations” in this context are usually amino acid substitutions, although as shown herein, deletions and insertions of amino acids can also be done and thus are defined as mutations.

The Fc variant antibodies of the disclosure show increased binding to FcRn and/or increased in vivo serum half-life. By “FcRn” or “neonatal Fc Receptor” as used herein is meant a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin. In some cases, the FcRn variants bind to the human FcRn receptor, or it may be desirable to design variants that bind to rodent or primate receptors in addition, to facilitate clinical trials.

In some embodiments, the present disclosure provides compositions and methods of administering an antibody to a subject, where the antibody comprises a variant Fc region as compared to a parent Fc region, wherein the variant Fc region comprises a first mutation that is a leucine at position 428 and a second mutation that is a serine at position 434, where the antibody has increased serum half-life as compared to an antibody comprising the parent Fc region, and wherein numbering is according to the EU index. In some embodiments, the antibody disclosed herein comprises a variant Fc region comprising mutations that substitute a methionine at position 428 with a leucine (Met428Leu) and substitute an asparagine at position 434 with a serine (Asn434Ser). Numbering is EU as in Kabat, and it is understood that the substitution is non-native to the starting molecule. As has been shown previously, these FcRn substitutions work in IgG1, IgG2 and IgG1/G2 hybrid backbones, and are specifically included for IgG3 and IgG4 backbones and derivatives of any IgG isoform as well.

The present disclosure includes variants of Fc domains, including those found in antibodies, Fc fusions, and immuno-adhesions, which have an increased binding to the FcRn receptor. As noted herein, binding to FcRn results in longer serum retention in vivo. A variety of such substitutions are known and described in U.S. Pat. Nos. 7,317,091; 8,084,582; and 8,101,720; 8,188,231; 8,367,805; and 8,546,543, each of which is incorporated herein by reference in its entirety.

### Dosing and Administration

The compound, antibody, or an antigen binding fragment thereof, can be administered in a single dose or in multiple doses. In some embodiments, the therapeutic antibody is administered to the subject in a single dose. In some embodiments, the therapeutic antibody is administered to the subject in multiple doses, spread out over the course of a few days, weeks or months. In some embodiments the antibody, or an antigen binding fragment thereof, is administered every week or every 2 weeks or every 3 weeks or every 4 weeks or every 5 weeks or every 6 weeks or every 7 weeks or every 8 weeks or every month or every 2 months or every 3 months.

In some embodiments the antibody, or an antigen binding fragment thereof, is administered in multiple doses and the dosage is the same each time. In some embodiments the antibody, or an antigen binding fragment thereof, is administered in multiple doses and the dosage at the time of first administration is different (could be higher or lower) from those at subsequent times. In some embodiments the antibody, or an antigen binding fragment thereof, is administered in multiple doses and the dosage is adjusted at each administration based on the subject's response to the therapy.

The dosage may further vary between patients, based on different factors such as the age, gender, race, and body weight of each patient. In some embodiments, the dosage varies by body weight of the patient. The dosage could range from about 1 mg of the antibody, or an antigen binding fragment thereof, per kilogram of body weight to about 100 mg of the antibody, or an antigen binding fragment thereof, per kilogram of body weight. The dosage, could for example, be 1 mg, 2 mg, 3 mg, 5 mg, 7 mg, 10 mg, 12 mg, 15 mg, 17 mg, 20 mg, 22 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg or 100 mg, of the antibody, or an antigen binding fragment thereof, per kilogram of body weight.

In some embodiments, the dose is about 0.3 mg/kg to about 10 mg/kg of the antibody, or an antigen binding fragment thereof. In some embodiments, the dosage is about 0.3 mg/kg to about 5 mg/kg of the antibody, or an antigen binding fragment thereof. In some embodiments, the dosage is about 0.3 mg/kg to about 1 mg/kg of the antibody, or an antigen binding fragment thereof. The dosage, could for example, be about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, 1.6 mg/kg, 1.7 mg/kg, 1.8 mg/kg, 1.9 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, or about 10 mg/kg, or any number of tenths of a mg/kg in between the foregoing, of the antibody, or an antigen binding fragment thereof. In some embodiments, the dose is administered every week.

In some embodiments, the dose is about 0.6 mg/kg to about 20 mg/kg of the antibody, or an antigen binding fragment thereof. In some embodiments, the dosage is about 0.6 mg/kg to about 5 mg/kg of the antibody, or an antigen binding fragment thereof. In some embodiments, the dosage is about 0.6 mg/kg to about 10 mg/kg of the antibody, or an antigen binding fragment thereof. The dosage, could for example, be about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, 1.6 mg/kg, 1.7 mg/kg, 1.8 mg/kg, 1.9 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg, or any number of tenths of a mg/kg in between the foregoing, of the antibody, or an antigen binding fragment thereof. In some embodiments, the dose is administered every two weeks.

In some embodiments, the dose is about 1 mg/kg to about 30 mg/kg of the antibody, or an antigen binding fragment thereof. In some embodiments, the dose is about 5 mg/kg to about 30 mg/kg of the antibody, or an antigen binding fragment thereof. In some embodiments, the dose is about 10 mg/kg to about 30 mg/kg of the antibody, or an antigen binding fragment thereof. The dosage, could for example, be about 1 mg/kg, about 2 mg/kg, about 3 mg/kg, about 5 mg/kg, about 7 mg/kg, about 10 mg/kg, about 12 mg/kg, about 15 mg/kg, about 17 mg/kg, about 20 mg/kg, about 22 mg/kg, about 25 mg/kg, or about 30 mg/kg, or any integer and/or number of tenths of a mg/kg in between the foregoing, of the antibody, or an antigen binding fragment thereof. In some embodiments, the dose is administered every three weeks.

In some embodiments, the dose is about 1.2 mg/kg to about 40 mg/kg of the antibody, or an antigen binding fragment thereof. In some embodiments, the dose is about 5 mg/kg to about 40 mg/kg of the antibody, or an antigen binding fragment thereof. In some embodiments, the dose is about 10 mg/kg to about 40 mg/kg of the antibody, or an antigen binding fragment thereof. In some embodiments, the dose is about 20 mg/kg to about 40 mg/kg of the antibody, or an antigen binding fragment thereof. In some embodiments, the dose is about 25 mg/kg to about 40 mg/kg of the antibody, or an antigen binding fragment thereof. The dosage, could for example, be about 1 mg/kg, about 2 mg/kg, about 3 mg/kg, about 5 mg/kg, about 7 mg/kg, about 10 mg/kg, about 12 mg/kg, about 15 mg/kg, about 17 mg/kg, about 20 mg/kg, about 22 mg/kg, about 25 mg/kg, about 27 mg/kg, about 30 mg/kg, about 32 mg/kg, about 35 mg/kg, about 37 mg/kg, or about 40 mg/kg, or any integer and/or number of tenths of a mg/kg in between the foregoing, of the antibody, or an antigen binding fragment thereof. In some embodiments, the dose is administered every four weeks.

In some embodiments, the dosage is about 1 mg/kg to about 5 mg/kg of the antibody, or an antigen binding fragment thereof. In some embodiments, the dosage is about 5 mg/kg to about 10 mg/kg of the antibody, or an antigen binding fragment thereof. In some embodiments, the dosage is about 10 mg/kg to about 15 mg/kg of the antibody, or an antigen binding fragment thereof. In some embodiments, the dosage is about 15 mg/kg to about 20 mg/kg of the antibody, or an antigen binding fragment thereof.

In some embodiments, the dosage is about 1 mg/kg to about 5 mg/kg of the antibody, or an antigen binding fragment thereof. In some embodiments, the dosage is about 5 mg/kg to about 10 mg/kg of the antibody, or an antigen binding fragment thereof. In some embodiments, the dosage is about 10 mg/kg to about 15 mg/kg of the antibody, or an antigen binding fragment thereof. In some embodiments, the dosage is about 15 mg/kg to about 20 mg/kg of the antibody, or an antigen binding fragment thereof.

In some embodiments the antibody, or an antigen binding fragment thereof, is administered in multiple doses and the dosage at the time of first administration is different from those at subsequent times, the dosage at the time of first administration is about 1 mg/kg to about 5 mg/kg of the antibody, or an antigen binding fragment thereof; or about 5 mg/kg to about 10 mg/kg of the antibody, or an antigen binding fragment thereof; or about 10 mg/kg to about 15 mg/kg of the antibody, or an antigen binding fragment thereof; or about 15 mg/kg to about 20 mg/kg of the antibody, or an antigen binding fragment thereof; or about 20 mg/kg to about 25 mg/kg of the antibody, or an antigen binding fragment thereof. The subsequent dose(s) could be higher or lower than the first dose. In some embodiments, the subsequent dose is about 1 mg/kg to about 5 mg/kg of the antibody, or an antigen binding fragment thereof; or about 5 mg/kg to about 10 mg/kg of the antibody, or an antigen binding fragment thereof; or about 10 mg/kg to about 15 mg/kg of the antibody, or an antigen binding fragment thereof; or about 15 mg/kg to about 20 mg/kg of the antibody, or an antigen binding fragment thereof; or about 20 mg/kg to about 25 mg/kg of the antibody, or an antigen binding fragment thereof.

Small molecule compounds may be administered orally, via injection, etc. at a dose of from 0.01 to 500 mg/kg per day and/or from 0.1 mg to 5 g per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, for example around 10 mg to 200 mg.

The amount of active ingredient that may be combined with the carrier materials to produce a single dosage form will vary depending upon the host treated and the particular mode of administration. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

Additional dosage ranges are provided throughout this disclosure.

The duration of the treatment depends on the subject's response to the therapy and can range from about one month or 4 weeks to about 2 years or 100 weeks. In some embodiments, the treatment may be provided over a total duration of about 1 month, 2 months, 3 months, 4 months, 5 months, 6 months, 7 months, 8 months, 9 months, 10 months, 11 months, 1 year, 14 months, 16 months, 18 months, 20 months, 22 months or 2 years. In some embodiments, the treatment may be provided over a total duration of 4, 6, 8, 10, 12, 14, 16, 20, 22, 24, 26, 28, 30, 32, 34, 36, 38, 40, 42, 44, 46, 48, 50, 52 weeks, or extended to 56, 64, 72, 80, 88, 96 or 104 weeks.

In some embodiments, the antibody, or an antigen binding fragment thereof, is administered for a duration of 24 weeks at intervals of 3 weeks starting with an initial dose of 10 mg per kilogram of body weight, followed by 20 mg per kilogram for seven additional treatments. In some embodiments, the slam molecule compound is administered daily (QD), twice daily, (BID) or thrice daily (TID) for an appropriate duration, e.g., 24 weeks.

The compound, antibody, or an antigen binding fragment thereof, may be administered by any suitable route including, but not limited to, oral, intravenous, intramuscular, intra-arterial, intramedullary, intraperitoneal, intrathecal, intraventricular, transdermal, transcutaneous, topical, subcutaneous, intranasal, enteral, sublingual, intravaginal or rectal routes. Hyposprays may also be used to administer the pharmaceutical compositions disclosed herein. Typically, the therapeutic antibody may be prepared as a freeze-dried (lyophilized) powder or as an injectable, either as a liquid solution or suspension. Solid forms suitable for solution in, or suspension in, liquid vehicles prior to injection may also be used.

### Pharmaceutical Compositions

The pharmaceutical compositions used in the methods disclosed herein comprise one or more of: the antibodies or antibody fragments described above and a pharmaceutically acceptable carrier or excipient. Although the carrier or excipient may facilitate administration, it should not itself induce the production of antibodies harmful to the subject or individual receiving the composition; nor should it be toxic. Suitable carriers may be large, slowly metabolized macromolecules such as proteins, polypeptides, liposomes, polysaccharides, polylactic acids, polyglycolic acids, polymeric amino acids, amino acid copolymers and inactive virus particles, and are known to one of skill in the art.

The antibodies, or an antigen binding fragments thereof, or pharmaceutical compositions used with the methods disclosed herein may be administered by any number of routes including, but not limited to, oral, intravenous, intramuscular, intra-arterial, intramedullary, intraperitoneal, intrathecal, intraventricular, transdermal, transcutaneous, topical, subcutaneous, intranasal, enteral, sublingual, intravaginal or rectal routes. Hyposprays may also be used to administer the pharmaceutical compositions disclosed herein. Typically, the therapeutic compositions may be prepared as injectables, either as liquid solutions or suspensions. Solid forms suitable for solution in, or suspension in, liquid vehicles prior to injection may also be prepared.

In one embodiment, the antibody, or an antigen binding fragment thereof, or pharmaceutical composition is administered intravenously. In another embodiment, the antibody, or an antigen binding fragment thereof, or pharmaceutical composition is administered by intravenous infusion.

Direct delivery of the compositions will generally be accomplished by injection, subcutaneously, intraperitoneally, intravenously or intramuscularly, or delivered to the interstitial space of a tissue. The compositions can also be administered into a lesion. Dosage treatment may be a single dose schedule or a multiple dose schedule. Known antibody-based pharmaceuticals provide guidance relating to frequency of administration e.g., whether a pharmaceutical should be delivered daily, weekly, monthly, etc. Frequency and dosage may also depend on the severity of symptoms.

It will be appreciated that the active ingredient in the composition will be an antibody molecule, an antibody fragment or variants and derivatives thereof. As such, it will be susceptible to degradation in the gastrointestinal tract. Thus, if the composition is to be administered by a route using the gastrointestinal tract, the composition will need to contain agents which protect the antibody from degradation, but which release the antibody once it has been absorbed from the gastrointestinal tract.

For larger molecular weight moieties such as mAbs (˜150 kDa), the SC capillaries have low passive permeability; absorption of mAbs into systemic circulation occurs via lymphatic uptake from the interstitial space, as well as via active transport by the neonatal Fc receptor (FcRn) across the capillary endothelia. The extracellular matrix of the subcutaneous tissue also limits the injection of larger volumes (>1-2 mL) SC generally; coformulation with a recombinant hyaluronidase or soluble fragment thereof such as rHuPH20 can permit higher bioavailability. Additionally, physiochemical properties of mAbs, including charge, hydrophobicity, and stability, affect the rate and extent of their SC absorption; for example, the combination of high positive charge and hydrophobic interaction can reduce the rate absorption.

In some embodiments, pharmaceutical compositions suitable for use in the methods disclosed herein are formulated for subcutaneous administration. Examples of formulations suitable for subcutaneous administration include, but are not limited to, solutions, suspensions, emulsions, and dry products that can be dissolved or suspended in a pharmaceutically acceptable carrier for injection. Antibodies have been, and may be, formulated for subcutaneous administration using methods known in the art.

Pharmaceutical compositions suitable for use in the methods disclose herein comprise one or more pharmaceutically acceptable carriers, such as those widely employed in the art of drug manufacturing, and particularly antibody drug manufacturing. Pharmaceutically acceptable carriers in particular are non-toxic and should not interfere with the efficacy of the active ingredient. The carrier may be a diluent, adjuvant, excipient, or vehicle with which the antibodies are administered. Such vehicles may be liquids, such as aqueous fluids, oils, and emulsions. For example, 0.4% saline and 0.3% glycine may be used. The solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the antibodies in such pharmaceutical formulation may vary and will be selected primarily based on required dose, fluid volumes, viscosities, etc., according to the particular mode of administration selected, and other concerns, such as protein aggregation.

Examples of pharmaceutically acceptable carriers are solvents, dispersion media, coatings, antibacterial and antifungal agents, isotonic and absorption delaying agents, and the like that are physiologically compatible, such as salts, buffers, antioxidants, saccharides, aqueous or non-aqueous carriers, preservatives, wetting agents, surfactants or emulsifying agents, permeation enhancers, or combinations thereof.

Examples of buffers that may be used are acetic acid, citric acid, formic acid, succinic acid, phosphoric acid, carbonic acid, malic acid, aspartic acid, histidine, boric acid, Tris buffers, HEPPSO and HEPES.

Examples of antioxidants that may be used are ascorbic acid, methionine, cysteine hydrochloride, sodium bisulfate, sodium metabisulfite, sodium sulfite, lecithin, citric acid, ethylenediamine tetraacetic acid (EDTA), sorbitol and tartaric acid.

Examples of amino acids that may be used are histidine, isoleucine, methionine, glycine, arginine, lysine, L-leucine, tri-leucine, alanine, glutamic acid, L-threonine, and 2-phenylamine.

Examples of surfactants that may be used are polysorbates (e.g., polysorbate-20 or polysorbate-80); polyoxamers (e.g, poloxamer 188); Triton; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g, lauroamidopropyl); myristamidopropyl-, palmidopropyl-, or isostearamidopropyl-dimethylamine; sodium methyl cocoyl-, or disodium methyl oleyl-taurate; and the MONAQUA™ series (Mona Industries, Inc., Paterson, N.J.), polyethyl glycol, polypropyl glycol, and copolymers of ethylene and propylene glycol (e.g PLURONICS™, PF68, etc.).

Examples of preservatives that may be used are phenol, m-cresol, p-cresol, o-cresol, chlorocresol, benzyl alcohol, phenylmercuric nitrite, phenoxyethanol, formaldehyde, chlorobutanol, magnesium chloride, alkylparaben (methyl, ethyl, propyl, butyl and the like), benzalkonium chloride, benzethonium chloride, sodium dehydroacetate and thimerosal, or mixtures thereof.

Examples of saccharides that may be used are monosaccharides, disaccharides, trisaccharides, polysaccharides, sugar alcohols, reducing sugars, nonreducing sugars such as glucose, sucrose, trehalose, lactose, fructose, maltose, dextran, glycerin, dextran, erythritol, glycerol, arabitol, sylitol, sorbitol, mannitol, mellibiose, melezitose, raffmose, mannotriose, stachyose, maltose, lactulose, maltulose, glucitol, maltitol, lactitol or iso-maltulose.

Examples of permeation enhancers that may be used include recombinant hyaluronidase or soluble fragment thereof such as rHuPH20 (Halozyme). Liquid formulations for subcutaneous administration may comprise rHuPH20 or another soluble human hyaluronidase enzyme. rHuPH20 may be present in an amount sufficient to result in an increase in the dispersion of the antibodies contained in the same liquid formulation during subcutaneous administration.

The amounts of pharmaceutically acceptable carrier(s) in the pharmaceutical compositions may be determined experimentally based on the activities of the carrier(s) and the desired characteristics of the formulation, such as stability, bioavailability, and/or minimal oxidation.

The methods of the present disclosure can use an antibody, or an antigen binding fragment thereof, as described above, alone or in combination with other pharmaceutically active compounds, to treat conditions such as those disclosed hereinabove. The additional pharmaceutically active compound(s) can be administered simultaneously (either in the same dosage form or in separate dosage forms) or sequentially. Accordingly, in one embodiment, the present disclosure comprises methods for treating a condition by administering to the subject a therapeutically-effective amount of an antibody, or an antigen binding fragment thereof, of the present disclosure and one or more additional pharmaceutically active compounds.

In some embodiments, the antibody, or an antigen binding fragment thereof, of the present disclosure is used in combination with existing therapies, including, but not limited to, corticosteroids; rituximab and other anti-CD20 antibodies; tocilizumab and other anti-IL-6 antibodies; or selenium, infliximab and other anti-TNF-alpha antibodies. In some embodiments, the antibody, or an antigen binding fragment thereof, of the present disclosure is used in combination with TSHR inhibitors.

### EXAMPLES

### Example A

### Teprotumumab

Provided first is teprotumumab (TEPEZZA), an IGF-1R inhibitor approved for the treatment of TED. Teprotumumab and other related IGF-1R inhibitor antibodies and their methods of preparation can be found in U.S. Pat. No. 7,572,897, US20190225696, and US20190270820, which are hereby incorporated by reference in their entireties. In certain embodiments, teprotumumab may be used as an active control in clinical trials of other IGF-1R inhibitors, e.g. as in Example 31.

TABLE A
Teprotumumab Sequences and SEQ ID Numbers

| SEQ | | |
|---|---|---|
| ID | | |
| NO | Description | Sequence |
| | Antibody 1 | |
| | (teprotumumab) | |
| 85 | CDRH1 aa | Ser Tyr Gly Met His |
| 86 | CDRH2 aa | He Ile Trp Phe Asp Gly Ser Ser Thr Tyr Tyr Ala Asp Ser Val Arg |
| | | Gly |
| 87 | CDRH3 aa | Glu Leu Gly Arg Arg Tyr Phe Asp Leu |
| 88 | CDRL1 aa | Arg Ala Ser Gln Ser Val Ser Ser Tyr Leu Ala |
| 89 | CDRL2 aa | Asp Ala Ser Lys Arg Ala Thr |
| 90 | CDRL3 aa | Gln Gln Arg Ser Lys Trp Pro Pro Trp Thr |
| 91 | VH aa | Gln Val Glu Leu Val Glu Ser Gly Gly Gly Val Val Gln Pro Gly |
| | | Arg Ser Gln Arg Leu Ser Cys Ala Ala Ser Gly Phe Thr Phe Ser |
| | | Ser Tyr Gly Met His Trp Val Arg Gln Ala Pro Gly Lys Gly Leu |
| | | Glu Trp Val Ala Ile Ile Trp Phe Asp Gly Ser Ser Thr Tyr Tyr Ala |
| | | Asp Ser Val Arg Gly Arg Phe Thr Ile Ser Arg Asp Asn Ser Lys |
| | | Asn Thr Leu Tyr Leu Gln Met Asn Ser Leu Arg Ala Glu Asp Thr |
| | | Ala Val Tyr Phe Cys Ala Arg Glu Leu Gly Arg Arg Tyr Phe Asp |
| | | Leu Trp Gly Arg Gly Thr Leu Val Ser Val Ser Ser |
| 92 | VL aa | Glu Ile Val Leu Thr Gln Ser Pro Ala Thr Leu Ser Leu Ser Pro |
| | | Gly Glu Arg Ala Thr Leu Ser Cys Arg Ala Ser Gln Ser Val Ser |
| | | Ser Tyr Leu Ala Trp Tyr Gln Gln Lys Pro Gly Gln Ala Pro Arg |
| | | Leu Leu Ile Tyr Asp Ala Ser Lys Arg Ala Thr Gly Ile Pro Ala |
| | | Arg Phe Ser Gly Ser Gly Ser Gly Thr Asp Phe Thr Leu Thr Ile |
| | | Ser Ser Leu Glu Pro Glu Asp Phe Ala Val Tyr Tyr Cys Gln Gln |
| | | Arg Ser Lys Trp Pro Pro Trp Thr Phe Gly Gln Gly Thr Lys Val |
| | | Glu Ser Lys |
| | Antibody 2 | |
| 85 | CDRH1 aa | Ser Tyr Gly Met His |
| 93 | CDRH2 aa | Ile Ile Trp Phe Asp Gly Ser Ser Lys Tyr Tyr Gly Asp Ser Val Lys |
| | | Gly |
| 87 | CDRH3 aa | Glu Leu Gly Arg Arg Tyr Phe Asp Leu |
| 88 | CDRL1 aa | Arg Ala Ser Gln Ser Val Ser Ser Tyr Leu Ala |
| 94 | CDRL2 aa | Asp Ala Ser Asn Arg Ala Thr |
| 89 | CDRL3 aa | Gln Gln Arg Ser Lys Trp Pro Pro Trp Thr |
| 95 | VH aa | Gln Val Gln Leu Val Glu Ser Gly Gly Gly Val Val Gln Pro Gly |
| | | Arg Ser Leu Arg Leu Ser Cys Ala Ala Ser Gly Phe Thr Phe Ser |
| | | Ser Tyr Gly Met His Trp Val Arg Gln Ala Pro Gly Lys Gly Leu |
| | | Glu Trp Met Ala Ile Ile Trp Phe Asp Gly Ser Ser Lys Tyr Tyr Gly |
| | | Asp Ser Val Lys Gly Arg Phe Thr Ile Ser Arg Asp Asn Ser Lys |
| | | Asn Thr Leu Tyr Leu Gln Met Asn Ser Leu Arg Ala Asp Asp Thr |
| | | Ala Val Tyr Tyr Cys Ala Arg Glu Leu Gly Arg Arg Tyr Phe Asp |
| | | Leu Trp Gly Arg Gly Thr Leu Val Thr Val Ser Ser |
| 96 | VL aa | Glu Ile Val Leu Thr Gln Ser Pro Ala Thr Leu Ser Leu Ser Pro |
| | | Gly Glu Arg Ala Thr Leu Ser Cys Arg Ala Ser Gln Ser Val Ser |
| | | Ser Tyr Leu Ala Trp Tyr Gln Gln Lys Pro Gly Gln Ala Pro Arg |
| | | Leu Leu Ile Tyr Asp Ala Ser Asn Arg Ala Thr Gly Ile Pro Ala |
| | | Arg Phe Ser Gly Ser Gly Ser Gly Thr Asp Phe Thr Leu Thr Ile |
| | | Ser Ser Leu Glu Pro Glu Asp Phe Ala Val Tyr Tyr Cys Gln Gln |
| | | Arg Ser Lys Trp Pro Pro Trp Thr Phe Gly Gln Gly Thr Lys Val |
| | | Glu Ile Lys |

### Example 1

### Dalotuzumab

Dalotuzumab and other related IGF-1R inhibitor antibodies and their methods of preparation can be found in WO 2005/058967, which is hereby incorporated by reference in its entirety.

Heavy Chain CDRs - Dalotuzumab

| HCDR1 | HCDR2 | HCDR3 |
|---|---|---|
| GGYLWN | YISYDGTNNYKPSLKD | YGRVFFDY |
| (SEQ ID NO: 1) | (SEQ ID NO: 2) | (SEQ ID NO: 3) |

Light Chain CDRs - Dalotuzumab

| LCDR1 | LCDR2 | LCDR3 |
|---|---|---|
| RSSQSIVHSNGNTYLQ | KVSNRLY | FQGSHVPWT |
| (SEQ ID NO: 4) | (SEQ ID NO: 5) | (SEQ ID NO: 6) |

| | |
|---|---|
| Heavy Chain | QVQLQESGPGLVKPSETLSLTCTVSGYSITGGYLWNWIRQPPGKGLEWIGYIS |
| (HC) | YDGTNNYKPSLKDRVTISRDTSKNQFSLKLSSVTAADTAVYYCARYGRVFF |
| | DYWGQGTLVTVSSASTKGPSVFPLAPSSKSTSGGTAALGCLVKDYFPEPVTV |
| | SWNSGALTSGVHTFPAVLQSSGLYSLSSVVTVPSSSLGTQTYICNVNHKPSNT |
| | KVDKRVEPKSCDKTHTCPPCPAPELLGGPSVFLFPPKPKDTLMISRTPEVTCV |
| | VVDVSHEDPEVKFNWYVDGVEVHNAKTKPREEQYNSTYRVVSVLTVLHQD |
| | WLNGKEYKCKVSNKALPAPIEKTISKAKGQPREPQVYTLPPSREEMTKNQVS |
| | LTCLVKGFYPSDIAVEWESNGQPENNYKTTPPVLDSDGSFFLYSKLTVDKSR |
| | WQQGNVFSCSVMHEALHNHYTQKSLSLSPGK (SEQ ID NO: 7) |
| Light Chain | DIVMTQSPLSLPVTPGEPASISCRSSQSIVHSNGNTYLQWYLQKPGQSPQLLIY |
| (LC) | KVSNRLYGVPDRFSGSGSGTDFTLKISRVEAEDVGVYYCFQGSHVPWTFGQ |
| | GTKVEIKRTVAAPSVFIFPPSDEQLKSGTASVVCLLNNFYPREAKVQWKVDN |
| | ALQSGNSQESVTEQDSKDSTYSLSSTLTLSKADYEKHKVYACE |
| | VTHQGLSSPVTKSFNRGEC (SEQ ID NO: 8) |

Some embodiments of the disclosure are anti-IGF-1R inhibitor mAbs or antigen binding fragments thereof, comprising a heavy chain comprising a variable heavy chain CDR1, a variable heavy chain CDR2, and a variable heavy chain CDR3, wherein the variable heavy chain CDR1 comprises an amino acid sequence SEQ ID NO:1, the variable heavy chain CDR2 comprises an amino acid sequence SEQ ID NO:2; and the variable heavy chain CDR3 comprises an amino acid sequence SEQ ID NO:3 or at least a CDR with at least 80% of sequence identity after optimal alignment with SEQ ID NO:1, SEQ ID NO:2, and SEQ ID NO:3.

The anti-IGF-1R inhibitor mAbs or antibody or antigen binding fragment thereof may additionally comprise a light chain which is paired with the heavy chain to form an antigen binding domain. In some embodiments, the light chain comprises a variable light chain CDR1, a variable light chain CDR2, and a variable light chain CDR3, wherein the variable light chain CDR1 comprises an amino acid sequence SEQ ID NO:4, the variable light chain CDR2 comprises an amino acid sequence SEQ ID NO:5; and the variable light chain CDR3 comprises an amino acid sequence SEQ ID NO:6 or at least a CDR with at least 80% of homology after optimal alignment with SEQ ID NO:4, SEQ ID NO:5, and SEQ ID NO:6.

In some embodiments, the anti-IGF-1R inhibitor mAbs or antigen binding fragment thereof comprises a heavy chain amino acid sequence of SEQ ID NO:7 or at least a heavy chain with at least 85%, 90%, 95%, 97%, 98%, or 99% of sequence identity after optimal alignment with SEQ ID NO:7. Alternatively, or in addition, the anti-IGF-1R inhibitor mAbs or antigen binding fragment thereof may comprise a light chain having an amino acid sequence of SEQ ID NO:8 or at least a heavy chain with at least 85%, 90%, 95%, 97%, 98%, or 99% of sequence identity after optimal alignment with SEQ ID NO:8.

### Example 2

### Ganitumab

Ganitumab and other related IGF-1R inhibitor antibodies and their methods of preparation can be found in WO 2006/069202, which is hereby incorporated by reference in its entirety.

Heavy Chain CDRs - Ganitumab

| HCDR1 | HCDR2 | HCDR3 |
|---|---|---|
| SSNWWS | EIYHSGSTNYNPSLKS | WTGRTDAFDI |
| (SEQ ID NO: 9) | (SEQ ID NO: 10) | (SEQ ID NO: 11) |

Light Chain CDRs - Ganitumab

| LCDR1 | LCDR2 | LCDR3 |
|---|---|---|
| ISCRSSQSLLHSNGYNYLD | LGSNRAS | MQGTHWPLT |
| (SEQ ID NO: 12) | (SEQ ID NO: 13) | (SEQ ID NO: 14) |

| | |
|---|---|
| Heavy Chain | QVQLQESGPGLVKPSGTLSLTCAVSGGSISSSNWWSWVRQPPGKGLEWIGEI |
| (HC) | YHSGSTNYNPSLKSRVTSVDKSKNQFSLKLSSVTAAD |
| | TAVYYCARWTGRTDAFDIWGQGTMVTVSSASTKGPSVFPL |
| | APSSKSTSGGTAALGCLVKDYFPEPVTVSWNSGALTSGVH |
| | TFPAVLQSSGLYSLSSVVTVPSSSLGTQTYICNVNHKPSN |
| | TKVDKKVEPKSCDKTHTCPPCPAPELLGGPSVFLFPPKPK DTLMISRTPE |
| | VTCVVVDVSH EDPEVKFNWYVDGVEVHNAK |
| | TKPREEQYNSTYRVVSVLTVLHQDWLNGKEYKCKVSNKAL |
| | PAPIEKTISKAKGQPREPQVYTLPPSRDELTKNQVSLTCL |
| | VKGFYPSDIAVEWESNGQPE NNYKTTPPVLDSDGSFFLYS |
| | KLTVDKSRWQQGNVFSCSVM HEALHNHYTQ KSLSLSPGK (SEQ ID NO: 15) |
| Light Chain | DVVMTQSPLS LPVTPGEPASISCRSSQSLLHSNGYNYLDW |
| (LC) | YLQKPGQSPQLLIYLGSNRASGVPDRFSGSGSGTDFTLKI |
| | SRVEAEDVGVYYCMQGTHWPLTFGQGTKVE IKRTVAAPSV |
| | FIFPPSDEQL KSGTASVVCL LNNFYPREAKVQWKVDNALQ SGNSQESVTE |
| | QDSKDSTYSLSSTLTLSKADYEKHKVYACE VTHQGLSSPVTKSFNRGEC |
| | (SEQ ID NO: 16) |

Some embodiments of the disclosure are anti-IGF-1R inhibitor mAbs or antigen binding fragments thereof, comprising a heavy chain comprising a variable heavy chain CDR1, a variable heavy chain CDR2, and a variable heavy chain CDR3, wherein the variable heavy chain CDR1 comprises an amino acid sequence SEQ ID NO:9, the variable heavy chain CDR2 comprises an amino acid sequence SEQ ID NO:10; and the variable heavy chain CDR3 comprises an amino acid sequence SEQ ID NO:11 or at least a CDR with at least 80% of sequence identity after optimal alignment with SEQ ID NO:9, SEQ ID NO:10, and SEQ ID NO:11.

The anti-IGF-1R inhibitor mAbs or antibody or antigen binding fragment thereof may additionally comprise a light chain which is paired with the heavy chain to form an antigen binding domain. In some embodiments, the light chain comprises a variable light chain CDR1, a variable light chain CDR2, and a variable light chain CDR3, wherein the variable light chain CDR1 comprises an amino acid sequence SEQ ID NO:12, the variable light chain CDR2 comprises an amino acid sequence SEQ ID NO:13; and the variable light chain CDR3 comprises an amino acid sequence SEQ ID NO:14 or at least a CDR with at least 80% of homology after optimal alignment with SEQ ID NO:12, SEQ ID NO:13, and SEQ ID NO:14.

In some embodiments, the anti-IGF-1R inhibitor mAbs or antigen binding fragment thereof comprises a heavy chain amino acid sequence of SEQ ID NO:15 or at least a heavy chain with at least 85%, 90%, 95%, 97%, 98%, or 99% of sequence identity after optimal alignment with SEQ ID NO:15. Alternatively, or in addition, the anti-IGF-1R inhibitor mAbs or antigen binding fragment thereof may comprise a light chain having an amino acid sequence of SEQ ID NO:16 or at least a heavy chain with at least 85%, 90%, 95%, 97%, 98%, or 99% of sequence identity after optimal alignment with SEQ ID NO:16.

### Example 3

### Xentuzumab

Xentuzumab and other related IGF-1R inhibitor antibodies and their methods of preparation can be found in WO 2014/135611, which is hereby incorporated by reference in its entirety.

Heavy Chain CDRs - Xentuzumab

| HCDR1 | HCDR2 | HCDR3 |
|---|---|---|
| SYWMS | SITSYGSFTYADSVK | NMYTHFDS |
| (SEQ ID NO: 17) | (SEQ ID NO: 18) | (SEQ ID NO: 19) |

Light Chain CDRs - Xentuzumab

| LCDR1 | LCDR2 | LCDR3 |
|---|---|---|
| SGSSSNIGSNSVS | DNSKRPS | QSRDTYGYYWV |
| (SEQ ID NO: 20) | (SEQ ID NO: 21) | (SEQ ID NO: 22) |
| QSRDTYGYYWV | | |

| | |
|---|---|
| Heavy Chain | QVELVESGGGLVQPGGSLRLSCAASGFTFTSYWMSWVRQA |
| (HC) | PGKGLELVSSITSYGSFTYYADSVKGRFTISRDNSKNTLY |
| | LQMNSLRAEDTAVYYCARNMYTHFDSWGQGTLVTVSSAST |
| | KGPSVFPLAPSSKSTSGGTAALGCLVKDYFPEPVTVSWNS GALTSGVHTF |
| | PAVLQSSGLYSLSSVVTVPS SSLGTQTYIC |
| | NVNHKPSNTKVDKKVEPKSCDKTHTCPPCPAPELLGGPSV |
| | FLFPPKPKDTLMISRTPEVTCVVVDVSHEDPEVKFNWYVD GVEVHNAKTK |
| | PREEQYNSTYRVVSVLTVLHQDWLNGKEYK |
| | CKVSNKALPAPIEKTISKAKGQPREPQVYTLPPSRDELTK NQVSLTCLVK |
| | GFYPSDIAVEWESNGQPENNYKTTPPVLDS (SEQ ID NO: 23) |
| Light Chain | DIVLTQPPSVSGAPGQRVTISCSGSSSNIGSNSVSWYQQL |
| (LC) | PGTAPKLLIYDNSKRPSGVPDRFSGSKSGTSASLAITGLQ |
| | SEDEADYYCQSRDTYGYYWVFGGGTKLTVLGQPKAAPSVT |
| | LFPPSSEELQANKATLVCLI SDFYPGAVTVAWKGDSSPVK |
| | AGVETTTPSKQSNNKYAASSYLSLTPEQWKSHRSYSCQVT |
| | HEGSTVEKTVAPTECS (SEQ ID NO: 24) |

Some embodiments of the disclosure are anti-IGF-1R inhibitor mAbs or antigen binding fragments thereof, comprising a heavy chain comprising a variable heavy chain CDR1, a variable heavy chain CDR2, and a variable heavy chain CDR3, wherein the variable heavy chain CDR1 comprises an amino acid sequence SEQ ID NO:17, the variable heavy chain CDR2 comprises an amino acid sequence SEQ ID NO:18; and the variable heavy chain CDR3 comprises an amino acid sequence SEQ ID NO:19 or at least a CDR with at least 80% of sequence identity after optimal alignment with SEQ ID NO:17, SEQ ID NO:18, and SEQ ID NO:19.

The anti-IGF-1R inhibitor mAbs or antibody or antigen binding fragment thereof may additionally comprise a light chain which is paired with the heavy chain to form an antigen binding domain. In some embodiments, the light chain comprises a variable light chain CDR1, a variable light chain CDR2, and a variable light chain CDR3, wherein the variable light chain CDR1 comprises an amino acid sequence SEQ ID NO:20, the variable light chain CDR2 comprises an amino acid sequence SEQ ID NO:21; and the variable light chain CDR3 comprises an amino acid sequence SEQ ID NO:22 or at least a CDR with at least 80% of homology after optimal alignment with SEQ ID NO:20, SEQ ID NO:21, and SEQ ID NO:22.

In some embodiments, the anti-IGF-1R inhibitor mAbs or antigen binding fragment thereof comprises a heavy chain amino acid sequence of SEQ ID NO:23 or at least a heavy chain with at least 85%, 90%, 95%, 97%, 98%, or 99% of sequence identity after optimal alignment with SEQ ID NO:23. Alternatively, or in addition, the anti-IGF-1R inhibitor mAbs or antigen binding fragment thereof may comprise a light chain having an amino acid sequence of SEQ ID NO:24 or at least a heavy chain with at least 85%, 90%, 95%, 97%, 98%, or 99% of sequence identity after optimal alignment with SEQ ID NO:24.

### Example 4

### AVE1642

AVE1642 and other related IGF-1R inhibitor antibodies and their methods of preparation can be found in WO 2003/106621 and/or U.S. Pat. No. 7,538,195, both of which are hereby incorporated by reference in their entirety.

Heavy Chain CDRs - AVE1642

| HCDR1 | HCDR2 | HCDR3 |
|---|---|---|
| SYWMH | EINPSNGRTNYNEKFKR | GRPDYYGSSKWYFDV |
| (SEQ ID NO: 25) | (SEQ ID NO: 26) | (SEQ ID NO: 27) |
| GYTFTSYWMH | EINPSNGRTN | GRPDYYGSSKWYFDV |
| (SEQ ID NO: 75) | (SEQ ID NO: 76) | (SEQ ID NO: 27) |
| SYWMH | EINPSNGRTN | GRPDYYGSSKWYFDV |
| (SEQ ID NO: 25) | (SEQ ID NO: 77) | (SEQ ID NO: 27) |
| SYWMH | EINPSNGRTNYNQKFQG | GRPDYYGSSKWYFDV |
| (SEQ ID NO: 25) | (SEQ ID NO: 78) | (SEQ ID NO: 27) |
| GYTFTSYWMH | EINPSNGRTNYNQKFQG | GRPDYYGSSKWYFDV |
| (SEQ ID NO: 75) | (SEQ ID NO: 78) | (SEQ ID NO: 27) |

Light Chain CDRs - AVE1642

| LCDR1 | LCDR2 | LCDR3 |
|---|---|---|
| RSSQSIVHSNVNTYLE | KVSNRFS | FQGSHVPPT |
| (SEQ ID NO: 28) | (SEQ ID NO: 29) | (SEQ ID NO: 30) |

Variable Domains - AVE1642

| | |
|---|---|
| Heavy Chain | QVQLQQSGAELVKPGASVKLSCKASGYTFTSYWMHWVKQRPGQGLEWIGE |
| (VH1) | INPSNGRTNYNEKFKRKATLTVDKSSSTAYMQLSSLTSEDSAVYYFARGRPD |
| | YYGSSKWYFDVWGAGTTVTVSS (SEQ ID NO: 31) |
| Heavy Chain | QVQLVQSGAEVVKPGASVKLSCKASGYTFTSYWMHWVKQRPGQGLEWIG |
| (VH2) | EINPSNGRTNYNQKFQGKATLTVDKSSSTAYMQLSSLTSEDSAVYYFARGRP |
| | DYYGSSKWYFDVWGQGTTVTVSS (SEQ ID NO: 79) |
| Heavy Chain | QVQLVQSGAEVVKPGASVKLSCKASGYTFTSYWMHWVKQRPGQGLEWIG |
| (VH3) | EINPSNGRTNYNQKFQGKATLTVDKSSSTAYMQLSSLTSEDSAVYYFARGRP |
| | DYYGSSKWYFDVWGQGTTVTVS (SEQ ID NO: 80) |
| Light Chain | DVLMTQTPLSLPVSLGDQASISCRSSQSIVHSNVNTYLEWYLQKPGQSPKLLI |
| (VL1) | YKVSNRFSGVPDRFSGSGSGTDFTLRISRVEAEDLGIYYCFQGSHVPPTFGGG |
| | TKLEIKR (SEQ ID NO: 32) |
| Light Chain | DVVMTQTPLSLPVSLGDPASISCRSSQSIVHSNVNTYLEWYLQKPGQSPRLLI |
| (VL2) | YKVSNRFSGVPDRFSGSGAGTDFTLRISRVEAEDLGIYYCFQGSHVPPTFGGG |
| | TKLEIKR (SEQ ID NO: 81) |
| Light Chain | DVLMTQTPLSLPVSLGDPASISCRSSQSIVHSNVNTYLEWYLQKPGQSPKLLI |
| (VL3) | YKVSNRFSGVPDRFSGSGAGTDFTLRISRVEAEDLGIYYCFQGSHVPPTFGGG |
| | TKLEIKR (SEQ ID NO: 82) |
| Light Chain | DVLMTQTPLSLPVSLGDPASISCRSSQSIVHSNVNTYLEWYLQKPGQSPRLLI |
| (VL4) | YKVSNRFSGVPDRFSGSGAGTDFTLRISRVEAEDLGIYYCFQGSHVPPTFGGG |
| | TKLEIKR (SEQ ID NO: 83) |
| Light Chain | DVVMTQTPLSLPVSLGDPASISCRSSQSIVHSNVNTYLEWYLQKPGQSPKLLI |
| (VL5) | YKVSNRFSGVPDRFSGSGAGTDFTLRISRVEAEDLGIYYCFQGSHVPPTFGGG |
| | TKLEIKR (SEQ ID NO: 84) |

Some embodiments of the disclosure are anti-IGF-1R inhibitor mAbs or antigen binding fragments thereof, comprising a heavy chain comprising a variable heavy chain CDR1, a variable heavy chain CDR2, and a variable heavy chain CDR3, wherein the variable heavy chain CDR1 comprises an amino acid sequence SEQ ID NO:25, the variable heavy chain CDR2 comprises an amino acid sequence SEQ ID NO:26; and the variable heavy chain CDR3 comprises an amino acid sequence SEQ ID NO:27 or at least a CDR with at least 80% of sequence identity after optimal alignment with SEQ ID NO:25, SEQ ID NO:26, and SEQ ID NO:27.

The anti-IGF-1R inhibitor mAbs or antibody or antigen binding fragment thereof may additionally comprise a light chain which is paired with the heavy chain to form an antigen binding domain. In some embodiments, the light chain comprises a variable light chain CDR1, a variable light chain CDR2, and a variable light chain CDR3, wherein the variable light chain CDR1 comprises an amino acid sequence SEQ ID NO:28, the variable light chain CDR2 comprises an amino acid sequence SEQ ID NO:29; and the variable light chain CDR3 comprises an amino acid sequence SEQ ID NO:30 or at least a CDR with at least 80% of homology after optimal alignment with SEQ ID NO:28, SEQ ID NO:29, and SEQ ID NO:30.

In some embodiments, the anti-IGF-1R inhibitor mAbs or antigen binding fragment thereof comprises a heavy chain amino acid sequence of SEQ ID NO:31 or at least a heavy chain with at least 85%, 90%, 95%, 97%, 98%, or 99% of sequence identity after optimal alignment with SEQ ID NO:31. Alternatively, or in addition, the anti-IGF-1R inhibitor mAbs or antigen binding fragment thereof may comprise a light chain having an amino acid sequence of SEQ ID NO:32 or at least a heavy chain with at least 85%, 90%, 95%, 97%, 98%, or 99% of sequence identity after optimal alignment with SEQ ID NO:32.

### Example 5

### Figitumumab

Figitumumab and other related IGF-1R inhibitor antibodies and their methods of preparation can be found in U.S. Pat. No. 7,037,498 which is hereby incorporated by reference in its entirety.

Heavy Chain CDRs - Figitumumab

| HCDR1 | HCDR2 | HCDR3 |
|---|---|---|
| GFTFSSYAMN | AISGSGGTTFYADSVKG | DLGWSDSYYYYYGMDV |
| (SEQ ID NO: 33) | (SEQ ID NO: 34) | (SEQ ID NO: 35) |

Light Chain CDRs - Figitumumab

| LCDR1 | LCDR2 | LCDR3 |
|---|---|---|
| RASQGIRNDLG | AASRLHR | LQHNSYPCS |
| (SEQ ID NO: 36) | (SEQ ID NO: 37) | (SEQ ID NO: 38) |

| | |
|---|---|
| Heavy Chain | EVQLLESGGGLVQPGGSLRLSCTASGFTFSSYAMNWVRQA |
| (HC) | PGKGLEWVSAISGSGGTTFYADSVKGRFTISRDNSRTTLY |
| | LQMNSLRAEDTAVYYCAKDLGWSDSYYYYYGMDVWGQGTT |
| | VTVSSASTKGPSVFPLAPCS RSTSESTAALGCLVKDYFPE |
| | PVTVSWNSGALTSGVHTFPAVLQSSGLYSLSSVVTVPSSN |
| | FGTQTYTCNVDHKPSNTKVD KTVERKCCVECPPCPAPPVA |
| | GPSVFLFPPKPKDTLMISRTPEVTCVVVDVSHEDPEVQFN |
| | WYVDGVEVHNAKTKPREEQFNSTFRVVSVLTVVHQDWLNG |
| | KEYKCKVSNKGLPAPIEKTI SKTKGQPREPQVYTLPPSRE |
| | EMTKNQVSLTCLVKGFYPSDIAVEWESNGQPENNYKTTPP |
| | MLDSDGSFFLYSKLTVDKSRWQQGNVFSCSVMHEALHNHY |
| | TQKSLSLSPGK (SEQ ID NO: 39) |
| Light Chain | DIQMTQFPSSLSASVGDRVTITCRASQGIRNDLGWYQQKPGKAPKRLIYAAS |
| (LC) | RLHRGVPSRFSGSGSGTEFTLTISSLQPEDFATYYCLQHNSYPCSFGQGTKLEI |
| | KRTVAAPSVFIFPPSDEQLKSGTASVVCLLNNFYPREAKVQWKVDNALQSG |
| | NSQESVTEQDSKDSTYSLSSTLTLSKADYEKHKVYACEVTHQGLSSPVTKSF |
| | NRGEC (SEQ ID NO: 40) |

Some embodiments of the disclosure are anti-IGF-1R inhibitor mAbs or antigen binding fragments thereof, comprising a heavy chain comprising a variable heavy chain CDR1, a variable heavy chain CDR2, and a variable heavy chain CDR3, wherein the variable heavy chain CDR1 comprises an amino acid sequence SEQ ID NO:33, the variable heavy chain CDR2 comprises an amino acid sequence SEQ ID NO:34; and the variable heavy chain CDR3 comprises an amino acid sequence SEQ ID NO:35 or at least a CDR with at least 80% of sequence identity after optimal alignment with SEQ ID NO:33, SEQ ID NO:34, and SEQ ID NO:35.

The anti-IGF-1R inhibitor mAbs or antibody or antigen binding fragment thereof may additionally comprise a light chain which is paired with the heavy chain to form an antigen binding domain. In some embodiments, the light chain comprises a variable light chain CDR1, a variable light chain CDR2, and a variable light chain CDR3, wherein the variable light chain CDR1 comprises an amino acid sequence SEQ ID NO:36, the variable light chain CDR2 comprises an amino acid sequence SEQ ID NO:37; and the variable light chain CDR3 comprises an amino acid sequence SEQ ID NO:38 or at least a CDR with at least 80% of homology after optimal alignment with SEQ ID NO:36, SEQ ID NO:37, and SEQ ID NO:38.

In some embodiments, the anti-IGF-1R inhibitor mAbs or antigen binding fragment thereof comprises a heavy chain amino acid sequence of SEQ ID NO:39 or at least a heavy chain with at least 85%, 90%, 95%, 97%, 98%, or 99% of sequence identity after optimal alignment with SEQ ID NO:39. Alternatively, or in addition, the anti-IGF-1R inhibitor mAbs or antigen binding fragment thereof may comprise a light chain having an amino acid sequence of SEQ ID NO:40 or at least a heavy chain with at least 85%, 90%, 95%, 97%, 98%, or 99% of sequence identity after optimal alignment with SEQ ID NO:40.

### Example 6

### Dusigitumab

Dusigitumab (MEDI-573) and other related IGF-1R inhibitor antibodies and their methods of preparation can be found in U.S. Pat. No. 7,939,637 which is hereby incorporated by reference in its entirety.

Heavy Chain CDRs - Dusigitumab

| HCDR1 | HCDR2 | HCDR3 |
|---|---|---|
| SYDIN | WMNPNSGNTGYAQKFQG | DPYYYYYGMDV |
| (SEQ ID NO: 41) | (SEQ ID NO: 42) | (SEQ ID NO: 43) |

Light Chain CDRs - Dusigitumab

| LCDR1 | LCDR2 | LCDR3 |
|---|---|---|
| SGSSSNIENNHVS | DNNKRPS | ETWDTSLSAGRV |
| (SEQ ID NO: 44) | (SEQ ID NO: 45) | (SEQ ID NO: 46) |

| | |
|---|---|
| Heavy Chain | QVQLVQSGAEVKKPGASVKVSCKASGYTFTSYDINWVRQ |
| (HC) | ATGQGLEWMGWMNPNSGNTGYAQKFQGRVTMTRNTSIST |
| | AYMELSSLRSEDTAVYYCARDPYYYYYGMDVWGQGTTVT |
| | VSSASTKGPSVFPLAPCSRSTSESTAALGCLVKDYFPEP |
| | VTVSWNSGALTSGVHTFPAVLQSSGLYSLSSVVTVPSSN |
| | FGTQTYTCNVDHKPSNTKVDKTVERKCCVECPPCPAPPV |
| | AGPSVFLFPPKPKDTLMISRTPEVTCVVVDVSHEDPEVQ |
| | FNWYVDGVEVHNAKTKPREEQFNSTFRVVSVLTVVHQDW |
| | LNGKEYKCKVSNKGLPAPIEKTISKTKGQPREPQVYTLP |
| | PSREEMTKNQVSLTCLVKGFYPSDIAVEWESNGQPENNY |
| | KTTPPMLDSDGSFFLYSKLTVDKSRWQQGNVFSCSVMHE |
| | ALHNHYTQKSLSLSPGK (SEQ ID NO: 47) |
| Light Chain | QSVLTQPPSVSAAPGQKVTISCSGSSSNIENNHVSWYQQ |
| (LC) | LPGTAPKLLIYDNNKRPSGIPDRFSGSKSGTSATLGITG |
| | LQTGDEADYYCETWDTSLSAGRVFGGGTKLTVLGQPKAA |
| | PSVTLFPPSSEELQANKATLVCLISDFYPGAVTVAWKAD |
| | SSPVKAGVETTTPSKQSNNKYAASSYLSLTPEQWKSHRS |
| | YSCQVTHEGSTVEKTVAPTECS (SEQ ID NO: 48) |

Some embodiments of the disclosure are anti-IGF-1R inhibitor mAbs or antigen binding fragments thereof, comprising a heavy chain comprising a variable heavy chain CDR1, a variable heavy chain CDR2, and a variable heavy chain CDR3, wherein the variable heavy chain CDR1 comprises an amino acid sequence SEQ ID NO:41, the variable heavy chain CDR2 comprises an amino acid sequence SEQ ID NO:42; and the variable heavy chain CDR3 comprises an amino acid sequence SEQ ID NO:43 or at least a CDR with at least 80% of sequence identity after optimal alignment with SEQ ID NO:41, SEQ ID NO:42, and SEQ ID NO:43.

The anti-IGF-1R inhibitor mAbs or antibody or antigen binding fragment thereof may additionally comprise a light chain which is paired with the heavy chain to form an antigen binding domain. In some embodiments, the light chain comprises a variable light chain CDR1, a variable light chain CDR2, and a variable light chain CDR3, wherein the variable light chain CDR1 comprises an amino acid sequence SEQ ID NO:44, the variable light chain CDR2 comprises an amino acid sequence SEQ ID NO:45; and the variable light chain CDR3 comprises an amino acid sequence SEQ ID NO:46 or at least a CDR with at least 80% of homology after optimal alignment with SEQ ID NO:44, SEQ ID NO:45, and SEQ ID NO:46.

In some embodiments, the anti-IGF-1R inhibitor mAbs or antigen binding fragment thereof comprises a heavy chain amino acid sequence of SEQ ID NO:39 or at least a heavy chain with at least 85%, 90%, 95%, 97%, 98%, or 99% of sequence identity after optimal alignment with SEQ ID NO:47. Alternatively, or in addition, the anti-IGF-1R inhibitor mAbs or antigen binding fragment thereof may comprise a light chain having an amino acid sequence of SEQ ID NO:40 or at least a heavy chain with at least 85%, 90%, 95%, 97%, 98%, or 99% of sequence identity after optimal alignment with SEQ ID NO:48.

### Example 7

### Cixutumumab

Cixutumumab and other related IGF-1R inhibitor antibodies and their methods of preparation can be found in U.S. Pat. No. 7,638,605 which is hereby incorporated by reference in its entirety.

Heavy Chain CDRs - Cixutumumab

| HCDR1 | HCDR2 | HCDR3 |
|---|---|---|
| SYAIS | GIIPIFGTANYAQKFQ | APLRFLEWSTQDHYYYYYM |
| (SEQ ID NO: 49) | (SEQ ID NO: 50) | VD (SEQ ID NO: 51) |

Light Chain CDRs - Cixutumumab

| LCDR1 | LCDR2 | LCDR3 |
|---|---|---|
| QGDSLRSYYAT | GENKRPS | KSRDGSGQHLV |
| (SEQ ID NO: 52) | (SEQ ID NO: 53) | (SEQ ID NO: 54) |

| | |
|---|---|
| Heavy Chain | EVQLVQSGAEVKKPGSSVKVSCKASGGTFSSYAISWV |
| (HC) | RQAPGQGLEWMGGIIPIFGTANYAQKFQGRVTITADK |
| | STSTAYMELSSLRSEDTAVYYCARAPLRFLEWSTQDH |
| | YYYYYMDVWGKGTTVTVSSASTKGPSVFPLAPSSKST |
| | SGGTAALGCLVKDYFPEPVTVSWNSGALTSGVHTFPA |
| | VLQSSGLYSLSSVVTVPSSSLGTQTYICNVNHKPSNT |
| | KVDKKVEPKSCDKTHTCPPCPAPELLGGPSVFLFPPK |
| | P DTLMISRTPEVTCVVVDVSHEDPEVKFNWYVDGVE |
| | VHNAKTKPREEQYNSTYRVVSVLTVLHQDWLNGKEYK |
| | CKVSNKALPAPIEKTISKAKGQPREPQVYTLPPSREE |
| | MTKNQVSLTCLVKGFYPSDIAVEWESNGQPENNYKTT |
| | PPVLDSDGSFFLYSKLTVDKSRWQQGNVFSCSVMHEA |
| | LHNHYTQKSLSLSPGK (SEQ ID NO: 55) |
| Light Chain | SSELTQDPAVSVALGQTVRITCQGDSLRSYYATWYQQ |
| (LC) | KPGQAPILVIYGENKRPSGIPDRFSGSSSGNTASLTI |
| | TGAQAEDEADYYCKSRDGSGQHLVFGGGTKLTVLGQ |
| | PKAAPSVTLFPPSSEELQANKATLVCLISDFYPGAVT |
| | VAWKADSSPVKAGVETTTPSKQSNNKYAASSYLSLTP |
| | EQWKSHRSYSCQVTHEGSTVEKTVAP AECS |
| | (SEQ ID NO: 56) |

Some embodiments of the disclosure are anti-IGF-1R inhibitor mAbs or antigen binding fragments thereof, comprising a heavy chain comprising a variable heavy chain CDR1, a variable heavy chain CDR2, and a variable heavy chain CDR3, wherein the variable heavy chain CDR1 comprises an amino acid sequence SEQ ID NO:49, the variable heavy chain CDR2 comprises an amino acid sequence SEQ ID NO:50; and the variable heavy chain CDR3 comprises an amino acid sequence SEQ ID NO:51 or at least a CDR with at least 80% of sequence identity after optimal alignment with SEQ ID NO:49, SEQ ID NO:50, and SEQ ID NO:51.

The anti-IGF-1R inhibitor mAbs or antibody or antigen binding fragment thereof may additionally comprise a light chain which is paired with the heavy chain to form an antigen binding domain. In some embodiments, the light chain comprises a variable light chain CDR1, a variable light chain CDR2, and a variable light chain CDR3, wherein the variable light chain CDR1 comprises an amino acid sequence SEQ ID NO:52, the variable light chain CDR2 comprises an amino acid sequence SEQ ID NO:53; and the variable light chain CDR3 comprises an amino acid sequence SEQ ID NO:54 or at least a CDR with at least 80% of homology after optimal alignment with SEQ ID NO:52, SEQ ID NO:53, and SEQ ID NO:54.

In some embodiments, the anti-IGF-1R inhibitor mAbs or antigen binding fragment thereof comprises a heavy chain amino acid sequence of SEQ ID NO:55 or at least a heavy chain with at least 85%, 90%, 95%, 97%, 98%, or 99% of sequence identity after optimal alignment with SEQ ID NO:55. Alternatively, or in addition, the anti-IGF-1R inhibitor mAbs or antigen binding fragment thereof may comprise a light chain having an amino acid sequence of SEQ ID NO:56 or at least a heavy chain with at least 85%, 90%, 95%, 97%, 98%, or 99% of sequence identity after optimal alignment with SEQ ID NO:56.

### Example 8

### BIIB022

BIIB022 and other related IGF-1R inhibitor antibodies and their methods of preparation can be found in U.S. Pat. No. 7,612,178 which is hereby incorporated by reference in its entirety.

Heavy Chain CDRs - BIIB022

| HCDR1 | HCDR2 | HCDR3 |
|---|---|---|
| IYRMQ | GISPSGGTTWYADSVKG | WSGGSGYAFDI |
| (SEQ ID NO: 57) | (SEQ ID NO: 58) | (SEQ ID NO: 59) |

Light Chain CDRs - BIIB022

| LCDR1 | LCDR2 | LCDR3 |
|---|---|---|
| QASRDIRNYN | DASSLQT | QQFDSLPHT |
| (SEQ ID NO: 60) | (SEQ ID NO: 61) | (SEQ ID NO: 62) |

| | |
|---|---|
| Heavy Chain | EVQLLESGGGLVQPGGSLRLSCAASGFTFSIYRMQWVRQ |
| (HC) | APGKGLEWVSGISPSGGTTWYADSVKGRFTISRDNSKNT |
| | LYLQMNSLRAEDTAVYYCARWSGGSGYAFDIWGQGTMVT |
| | VSS(SEQ ID NO: 63) |
| Light Chain | DIQMTQSPLSLSASVGDRVTITCQASRDIRNYLNWYQQK |
| (LC) | PGKAPKLLIYDASSLQTGVPSRFGGSGSGTDFSFTIGSL |
| | QPEDIATYYCQQFDSLPHTFGQGTKLEIK |
| | (SEQ ID NO: 64) |

Some embodiments of the disclosure are anti-IGF-1R inhibitor mAbs or antigen binding fragments thereof, comprising a heavy chain comprising a variable heavy chain CDR1, a variable heavy chain CDR2, and a variable heavy chain CDR3, wherein the variable heavy chain CDR1 comprises an amino acid sequence SEQ ID NO:57, the variable heavy chain CDR2 comprises an amino acid sequence SEQ ID NO:58; and the variable heavy chain CDR3 comprises an amino acid sequence SEQ ID NO:59 or at least a CDR with at least 80% of sequence identity after optimal alignment with SEQ ID NO:57, SEQ ID NO:58, and SEQ ID NO:59.

The anti-IGF-1R inhibitor mAbs or antibody or antigen binding fragment thereof may additionally comprise a light chain which is paired with the heavy chain to form an antigen binding domain. In some embodiments, the light chain comprises a variable light chain CDR1, a variable light chain CDR2, and a variable light chain CDR3, wherein the variable light chain CDR1 comprises an amino acid sequence SEQ ID NO:60, the variable light chain CDR2 comprises an amino acid sequence SEQ ID NO:61; and the variable light chain CDR3 comprises an amino acid sequence SEQ ID NO:62 or at least a CDR with at least 80% of homology after optimal alignment with SEQ ID NO:60, SEQ ID NO:61, and SEQ ID NO:62.

In some embodiments, the anti-IGF-1R inhibitor mAbs or antigen binding fragment thereof comprises a heavy chain amino acid sequence of SEQ ID NO:63 or at least a heavy chain with at least 85%, 90%, 95%, 97%, 98%, or 99% of sequence identity after optimal alignment with SEQ ID NO:63. Alternatively, or in addition, the anti-IGF-1R inhibitor mAbs or antigen binding fragment thereof may comprise a light chain having an amino acid sequence of SEQ ID NO:64 or at least a heavy chain with at least 85%, 90%, 95%, 97%, 98%, or 99% of sequence identity after optimal alignment with SEQ ID NO:64.

### Example 9

### Robatumumab

Heavy Chain (HC) and Light Chain (LC) for Robatumumab

| | |
|---|---|
| Heavy Chain | EVQLVQSGGG LVKPGGSLRL SCAASGFTFS SFAMHWVRQA |
| (HC) | PGKGLEWISV IDTRGATYYADSVKGRFTIS RDNAKNSLYL QMNSLRAEDT |
| | AVYYCARLGN FYYGMDVWGQ GTTVTVSSAS |
| | TKGPSVFPLA PSSKSTSGGT AALGCLVKDY FPEPVTVSWN SGALTSGVHT |
| | FPAVLQSSGLYSLSSVVTVP SSSLGTQTYI CNVNHKPSNT KVDKKVEPKS |
| | CDKTHTCPPC PAPELLGGPS |
| | VFLFPPKPKD TLMISRTPEV TCVVVDVSHE DPEVKFNVVYV |
| | DGVEVHNAKT KPREEQYNSTYRVVSVLTVL HQDWLNGKEY |
| | KCKVSNKALP APIEKTISKA KGQPREPQVY TLPPSRDELT |
| | KNQVSLTCLV KGFYPSDIAV EWESNGQPEN NYKTTPPVLD SDGSFFLYSK |
| | LTVDKSRWQQGNVFSCSVMH EALHNHYTQK SLSLSPGK (SEQ ID NO: 65) |
| Light Chain | EIVLTQSPGTLSVSPGERATLSCRASQSIGSSLHWYQQKPGQAPRLLIKYASQ |
| (LC) | SLSGIPDRFSGSGSGTDFTLTISRLEPEDFAVYYCHQSSRLPHTFGQGTKVEIK |
| | RTVAAPSVFIFPPSDEQLKSGTASVVCLLNNFYPREAKVQWKVDNALQSGNS |
| | QESVTEQDSKDSTYSLSSTLTLSKADYEKHKVYACEVTHQGLSSPVTKSFNR |
| | GEC (SEQ ID NO: 66) |

In some embodiments, the anti-IGF-1R inhibitor mAbs or antigen binding fragment thereof comprises a heavy chain amino acid sequence of SEQ ID NO:65 or at least a heavy chain with at least 85%, 90%, 95%, 97%, 98%, or 99% of sequence identity after optimal alignment with SEQ ID NO:65. Alternatively, or in addition, the anti-IGF-1R inhibitor mAbs or antigen binding fragment thereof may comprise a light chain having an amino acid sequence of SEQ ID NO:66 or at least a heavy chain with at least 85%, 90%, 95%, 97%, 98%, or 99% of sequence identity after optimal alignment with SEQ ID NO:66.

In some embodiments, said IGF-1R inhibitor is a small molecule.

### Example 10

### Linsitinib

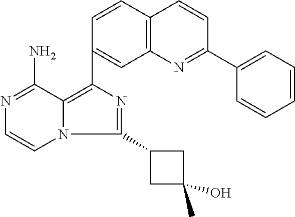 [SMILES: C[C@]1(O)C[C@@H](c2nc(-c3ccc4ccc(-c5ccccc5)nc4c3)c3c(N)nccn32)C1]

Linsitinib and other related IGF-1R inhibitor small molecules and their methods of preparation can be found in U.S. Pat. No. 8,101,613, which is hereby incorporated by reference in its entirety. Linsitinib and the other IGF-1R inhibitors described therein are predicted to have activity in the activity measures or assessments for the treatment of TED described herein.

### Example 11

### Picropodophyllin

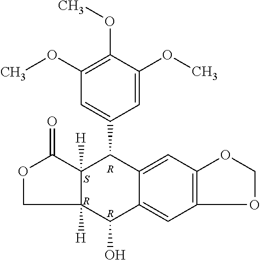

Picropodophyllin (AXL1717) and other related IGF-1R inhibitor small molecules and their methods of preparation can be found in US U.S. Pat. No. 4,567,253, which is hereby incorporated by reference in its entirety. Picropodophyllin and the other IGF-1R inhibitors described therein are predicted to have activity in the activity measures or assessments for the treatment of TED described herein.

### Example 12

### GTX-134

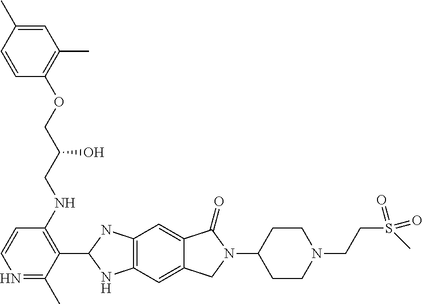 [SMILES: Cc1ccc(OC[C@H](O)CNc2ccnc(C)c2C2Nc3cc4c(cc3N2)C(=O)N(C2CCN(CCS(C)(=O)=O)CC2)C4)c(C)c1]

GTX-134 and other related IGF-1R inhibitor small molecules and their methods of preparation can be found in U.S. Pat. No. 8,063,225, which is hereby incorporated by reference in its entirety. GTX-134 and the other IGF-1R inhibitors described therein are predicted to have activity in the activity measures or assessments for the treatment of TED described herein.

### Example 13

### AG1024

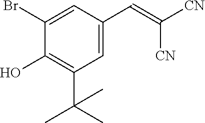 [SMILES: CC(C)(C)c1cc(C=C(C#N)C#N)cc(Br)c1O]

AG1024 and other related IGF-1R inhibitor small molecules and their methods of preparation can be found in WO1995024190, which is hereby incorporated by reference in its entirety. AG1024 and the other IGF-1R inhibitors described therein are predicted to have activity in the activity measures or assessments for the treatment of TED described herein.

### Example 14

### BMS-536924

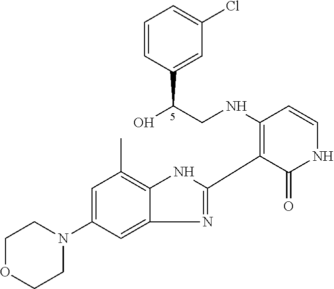 [SMILES: Cc1cc(N2CCOCC2)cc2nc(-c3c(NC[C@@H](O)c4cccc(Cl)c4)cc[nH]c3=O)[nH]c12]

BMS-536924 and other related IGF-1R inhibitor small molecules and their methods of preparation can be found in U.S. Pat. No. 7,081,454, which is hereby incorporated by reference in its entirety. BMS-536924 and the other IGF-1R inhibitors described therein are predicted to have activity in the activity measures or assessments for the treatment of TED described herein.

### Example 15

### NVP-AEW541

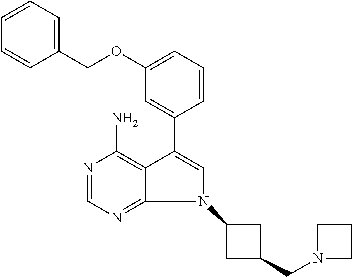 [SMILES: Nc1ncnc2c1c(-c1cccc(OCc3ccccc3)c1)cn2[C@H]1C[C@@H](CN2CCC2)C1]

NVP-AEW541 and other related IGF-1R inhibitor small molecules and their methods of preparation can be found in U.S. Pat. No. 7,326,699, which is hereby incorporated by reference in its entirety. NVP-AEW541 and the other IGF-1R inhibitors described therein are predicted to have activity in the activity measures or assessments for the treatment of TED described herein.

### Example 16

### BMS-754807

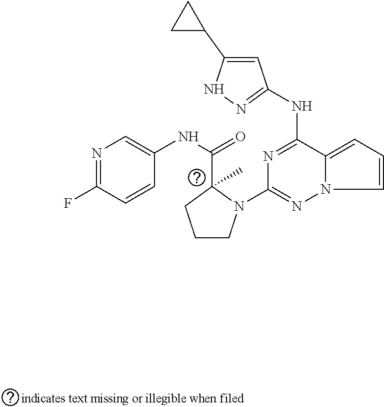 [SMILES: C[C@@]1(C(=O)Nc2ccc(F)nc2)CCCN1c1nc(Nc2cc(C3CC3)[nH]n2)c2cccn2n1]

BMS-754807 and other related IGF-1R inhibitor small molecules and their methods of preparation can be found in U.S. Pat. No. 7,534,792, which is hereby incorporated by reference in its entirety. BMS-754807 and the other IGF-1R inhibitors described therein are predicted to have activity in the activity measures or assessments for the treatment of TED described herein.

### Example 17

### GSK1838705A

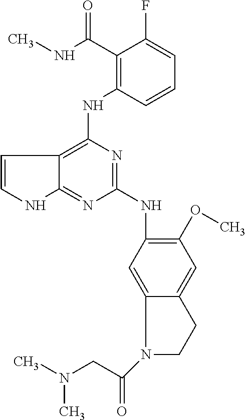 [SMILES: CNC(=O)c1c(F)cccc1Nc1nc(Nc2cc3c(cc2OC)CCN3C(=O)CN(C)C)nc2[nH]ccc12]

GSK1838705A and other related IGF-1R inhibitor small molecules and their methods of preparation can be found in U.S. Pat. No. 7,981,903, which is hereby incorporated by reference in its entirety. GSK1838705A and the other IGF-1R inhibitors described therein are predicted to have activity in the activity measures or assessments for the treatment of TED described herein.

### Example 18

### BMS-554417

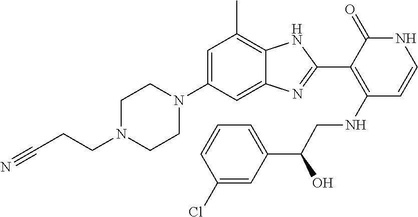 [SMILES: Cc1cc(N2CCN(CCC#N)CC2)cc2nc(-c3c(NC[C@@H](O)c4cccc(Cl)c4)cc[nH]c3=O)[nH]c12]

BMS-554417 and other related IGF-1R inhibitor small molecules and their methods of preparation can be found in U.S. Pat. No. 7,081,454, which is hereby incorporated by reference in its entirety. BMS-554417 and the other IGF-1R inhibitors described therein are predicted to have activity in the activity measures or assessments for the treatment of TED described herein.

### Example 19

### NVP-ADW742

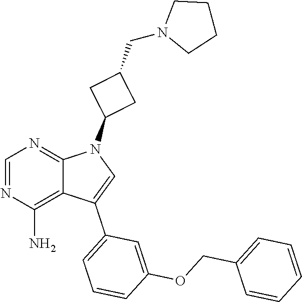 [SMILES: Nc1ncnc2c1c(-c1cccc(OCc3ccccc3)c1)cn2[C@H]1C[C@H](CN2CCCC2)C1]

NVP-ADW742 and other related IGF-1R inhibitor small molecules and their methods of preparation can be found in U.S. Pat. No. 7,326,699, which is hereby incorporated by reference in its entirety. NVP-ADW742 and the other IGF-1R inhibitors described therein are predicted to have activity in the activity measures or assessments for the treatment of TED described herein.

### Example 20

### GSK1904529A

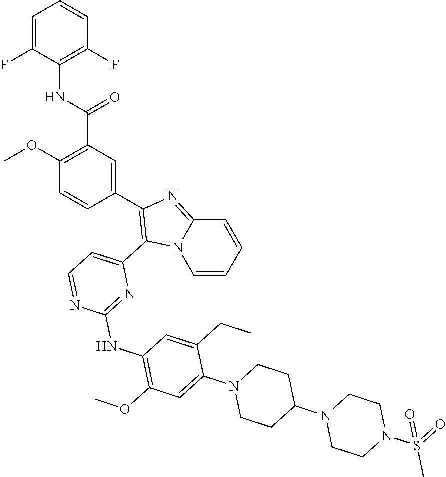 [SMILES: CCc1cc(Nc2nccc(-c3c(-c4ccc(OC)c(C(=O)Nc5c(F)cccc5F)c4)nc4ccccn34)n2)c(OC)cc1N1CCC(N2CCN(S(C)(=O)=O)CC2)CC1]

GSK1904529A and other related IGF-1R inhibitor small molecules and their methods of preparation can be found in U.S. Pat. No. 8,093,239, which is hereby incorporated by reference in its entirety. GSK1904529A and the other IGF-1R inhibitors described therein are predicted to have activity in the activity measures or assessments for the treatment of TED described herein.

### Example 21

### KW-2450

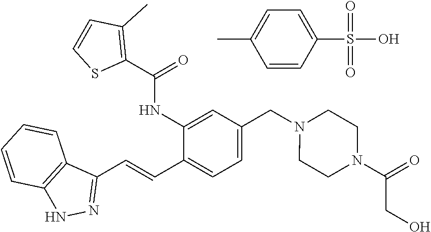

KW-2450, shown above as the tosylate salt but not limited thereto, and other related IGF-1R inhibitor small molecules and their methods of preparation can be found in WO2006080450, U.S. Pat. No. 7,605,272, and WO2011158931, which are hereby incorporated by reference in their entireties. KW-2450 and the other IGF-1R inhibitors described herein are predicted to have activity in the activity measures or assessments for the treatment of TED described herein.

### Example 22

### PL-225B

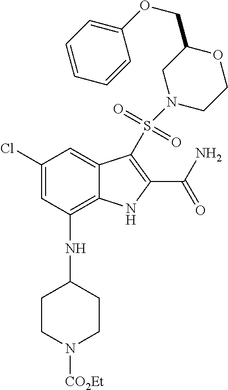 [SMILES: CCOC(=O)N1CCC(Nc2cc(Cl)cc3c(S(=O)(=O)N4CCO[C@H](COc5ccccc5)C4)c(C(N)=O)[nH]c23)CC1]

PL-225B and other related IGF-1R inhibitor small molecules and their methods of preparation can be found in WO2012145471 and WO2012007926, which is hereby incorporated by reference in its entirety. PL225B selectively inhibits IGF-1 R, resulting in inhibition of tumor cell proliferation and the induction of tumor cell apoptosis in IGF-1 R-overexpressing tumor cells. PL-225B and the other IGF-1R inhibitors described herein are predicted to have activity in the activity measures or assessments for the treatment of TED described herein.

### Example 23

### INSM-18, Nordihydroguaiaretic Acid (NDGA)/Masoprocol, Actinex

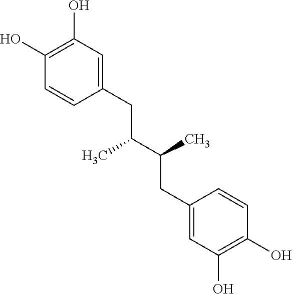 [SMILES: C[C@H](Cc1ccc(O)c(O)c1)[C@@H](C)Cc1ccc(O)c(O)c1]

INSM-18, nordihydroguaiaretic acid (NDGA) (shown above with relative stereochemistry, in which case it is also referred to as Masoprocol or Actinex, but not limited thereto) referred to in this Example as INSM-18, and other related IGF-1R inhibitor small molecules and their methods of preparation can be found at least in U.S. Pat. No. 2,373,192, which is hereby incorporated by reference in its entirety. INSM-18 directly inhibits activation of IGF-1 R and the c-erbB2/HER2/neu receptor, resulting in decreased proliferation of susceptible tumor cell populations. INSM-18 and the other IGF-1R inhibitors described herein are predicted to have activity in the activity measures or assessments for the treatment of TED described herein.

### Example 24

### AZD3463

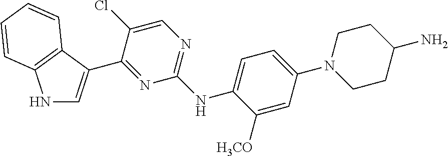 [SMILES: COc1cc(N2CCC(N)CC2)ccc1Nc1ncc(Cl)c(-c2c[nH]c3ccccc23)n1]

AZD3463 and other related IGF-1R inhibitor small molecules and their methods of preparation can be found in U.S. Pat. No. 8,461,170, which is hereby incorporated by reference in its entirety. AZD3463 is a potent ALK/IGF-1 R inhibitor, resulting in inhibition of neuroblastoma growth by overcoming crizotinib resistance and inducing apoptosis. AZD3463 and the other IGF-1R inhibitors described herein are predicted to have activity in the activity measures or assessments for the treatment of TED described herein.

### Example 25

### AZD9362

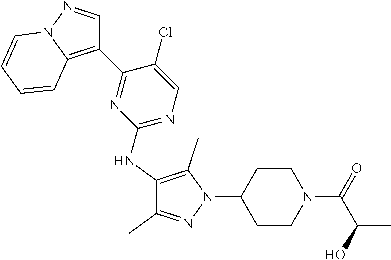 [SMILES: Cc1nn(C2CCN(C(=O)[C@@H](C)O)CC2)c(C)c1Nc1ncc(Cl)c(-c2cnn3ccccc23)n1]

AZD9362 and other related IGF-1R inhibitor small molecules and their methods of preparation can be found in Degorce, S L et al., “Discovery of a Potent, Selective, Orally Bioavailable, and Efficacious Novel 2-(Pyrazol-4-ylamino)-pyrimidine Inhibitor of the Insulin-like Growth Factor-1 Receptor (IGF-1R),” J Med Chem (2016), 59(10), 4859-4866., which is hereby incorporated by reference in its entirety. AZD9362 is a dual inhibitor of IGF-1R/InsR. AZD9362 and the other IGF-1R inhibitors described herein are predicted to have activity in the activity measures or assessments for the treatment of TED described herein.

### Example 26

### B1885578

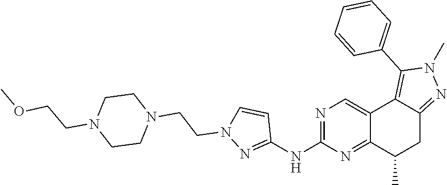

BI885578 and other related IGF-1R inhibitor small molecules and their methods of preparation can be found in U.S. Ser. No. 10/414,769, U.S. Pat. No. 9,150,578, and Sanderson M P et al., “BI 885578, a Novel IGF1R/INSR Tyrosine Kinase Inhibitor with Pharmacokinetic Properties That Dissociate Antitumor Efficacy and Perturbation of Glucose Homeostasis,” Mol Cancer Ther 2015 December; 14(12):2762-72, which are hereby incorporated by reference in its entirety. BI885578 is an IGF1R/INSR tyrosine kinase inhibitor distinguished by rapid intestinal absorption and a short in vivo half-life as a result of rapid metabolic clearance, resulting in inhibition of cell proliferation and induction of apoptosis in tumors. BI885578 and the other IGF-1R inhibitors described herein are predicted to have activity in the activity measures or assessments for the treatment of TED described herein.

### Example 27

### B1893923

BI893923 and other related IGF-1R inhibitor small molecules and their methods of preparation can be found in U.S. Pat. No. 8,546,443 and Titze M I et al., “An allometric pharmacokinetic/pharmacodynamics model for BI893923, a novel IGF-1 receptor inhibitor,” Cancer Chemother Pharmacol 2017 March; 79(3):545-558, which is hereby incorporated by reference in its entirety. BI893923 is an IGF1R/INSR tyrosine kinase inhibitor demonstrating anti-tumor efficacy and good tolerability. BI893923 and the other IGF-1R inhibitors described herein are predicted to have activity in the activity measures or assessments for the treatment of TED described herein.

### Example 28

### XL-228

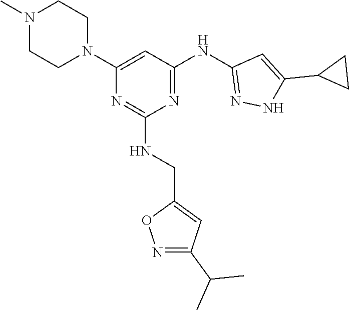 [SMILES: CC(C)c1cc(CNc2nc(Nc3cc(C4CC4)[nH]n3)cc(N3CCN(C)CC3)n2)on1]

XL-228 and other related IGF-1R inhibitor small molecules and their methods of preparation can be found in US20090232828, which is hereby incorporated by reference in its entirety. XL-228 is a broad protein kinase inhibitor that contributes to cell proliferation, cell survival, and resistance to cytotoxic agents. XL-228 and the other IGF-1R inhibitors described herein are predicted to have activity in the activity measures or assessments for the treatment of TED described herein.

### Example 29

### A-928605

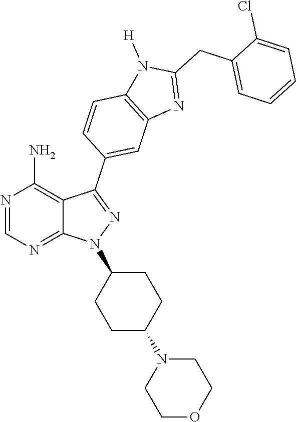 [SMILES: Nc1ncnc2c1c(-c1ccc3[nH]c(Cc4ccccc4Cl)nc3c1)nn2[C@H]1CC[C@H](N2CCOCC2)CC1]

A-928605 and other related IGF-1R inhibitor small molecules and their methods of preparation can be found in U.S. Pat. No. 7,772,231 and WO2007079164, which are hereby incorporated by reference in its entirety. A-928605 is a potent inhibitor of IGF-IR both on the purified enzyme and intracellular IGF-IR phosphorylation. A-928605 and the other IGF-1R inhibitors described herein are predicted to have activity in the activity measures or assessments for the treatment of TED described herein.

### Example 30

### Istiratumab (MM-141)

Istiratumab and other related IGF-1R inhibitor antibodies and their methods of preparation can be found in U.S. Pat. No. 8,476,409, which is hereby incorporated by reference in its entirety.

Heavy Chain CDRs - Istiratumab

| HCDR1 | HCDR2 | HCDR3 |
|---|---|---|
| GFMFSRYPMH | ISGSGGATPYADSVKG | DFYQILTGNAFDY |
| (SEQ ID NO: 67) | (SEQ ID NO: 68) | (SEQ ID NO: 69) |

Light Chain CDRs - Istiratumab

| LCDR1 | LCDR2 | LCDR3 |
|---|---|---|
| RASQGISSYLA | AKSTLQS | QQYWTFPLT |
| (SEQ ID NO: 70) | (SEQ ID NO: 71) | (SEQ ID NO: 72) |

| | |
|---|---|
| Heavy Chain | EVQLLQSGGGLVQPGGSLRLSCAASGFMFSRYPMHWVRQ |
| (HC) | APGKGLEWVGSISGSGGATPYADSVKGRFTISRDNSKNT |
| | LYLQMNSLRAEDTAVYYCAKDFYQILTGNAFDYWGQGTT |
| | VTVSSASTKGPSVFPLAPSSKSTSGGTAALGCLVKDYFP |
| | EPVTVSWNSGALTSGVHTFPAVLQSSGLYSLSSVVTVPS |
| | SSLGTQTYICNVNHKPSNTKVDKKVEPKSCDKTHTCPPC |
| | PAPELLGGPSVFLFPPKPKDTLMISRTPEVTCVVVDVSH |
| | EDPEVKFNWYVDGVEVHNAKTKPREEQYNSTYRVVSVLT |
| | VLHQDWLNGKEYKCKVSNKALPAPIEKTISKAKGQPREP |
| | QVYTLPPSREEMTKNQVSLTCLVKGFYPSDIAVEWESNG |
| | QPENNYKTTPPVLDSDGSFFLYSKLTVDKSRWQQGNVFS |
| | CSVMHEALHNHYTQKSLSLSPGGGGGSGGGGSGGGGSQV |
| | QLVQSGGGLVQPGGSLRLSCAASGFTFDDYAMHWVRQAP |
| | GKGLEWVAGISWDSGSTGYADSVKGRFTISRDNAKNSLY |
| | LQMNSLRAEDTALYYCARDLGAYQWVEGFDYWGQGTLVT |
| | VSSASTGGGGSGGGGSGGGGSGGGGSSYELTQDPAVSVA |
| | LGQTVRITCQGDSLRSYYASWYQQKPGQAPVLVIYGKNN |
| | RPSGIPDRFSGSTSGNSASLTITGAQAEDEADYYCNSRD |
| | SPGNQWVFGGGTKVTVLG |
| | (SEQ ID NO: 73) |
| Light Chain | DIQMTQSPSSLSASLGDRVTITCRASQGISSYLAWYQQK |
| (LC) | PGKAPKLLIYAKSTLQSGVPSRFSGSGSGTDFTLTISSL |
| | QPEDSATYYCQQYWTFPLTFGGGTKVEIKRTVAAPSVFI |
| | FPPSDEQLKSGTASVVCLLNNFYPREAKVQWKVDNALQS |
| | GNSQESVTEQDSKDSTYSLSSTLTLSKADYEKHKVYACE |
| | VTHQGLSSPVTKSFNRGEC |
| | (SEQ ID NO: 74) |

Some embodiments of the disclosure are anti-IGF-1R inhibitor mAbs or antigen binding fragments thereof, comprising a heavy chain comprising a variable heavy chain CDR1, a variable heavy chain CDR2, and a variable heavy chain CDR3, wherein the variable heavy chain CDR1 comprises an amino acid sequence SEQ ID NO:67, the variable heavy chain CDR2 comprises an amino acid sequence SEQ ID NO:68; and the variable heavy chain CDR3 comprises an amino acid sequence SEQ ID NO:69 or at least a CDR with at least 80% of sequence identity after optimal alignment with SEQ ID NO:67, SEQ ID NO:68, and SEQ ID NO:69.

The anti-IGF-1R inhibitor mAbs or antibody or antigen binding fragment thereof may additionally comprise a light chain which is paired with the heavy chain to form an antigen binding domain. In some embodiments, the light chain comprises a variable light chain CDR1, a variable light chain CDR2, and a variable light chain CDR3, wherein the variable light chain CDR1 comprises an amino acid sequence SEQ ID NO:70, the variable light chain CDR2 comprises an amino acid sequence SEQ ID NO:71; and the variable light chain CDR3 comprises an amino acid sequence SEQ ID NO:72 or at least a CDR with at least 80% of homology after optimal alignment with SEQ ID NO:70, SEQ ID NO:71, and SEQ ID NO:72.

In some embodiments, the anti-IGF-1R inhibitor mAbs or antigen binding fragment thereof comprises a heavy chain amino acid sequence of SEQ ID NO:73 or at least a heavy chain with at least 85%, 90%, 95%, 97%, 98%, or 99% of sequence identity after optimal alignment with SEQ ID NO:73. Alternatively, or in addition, the anti-IGF-1R inhibitor mAbs or antigen binding fragment thereof may comprise a light chain having an amino acid sequence of SEQ ID NO:74 or at least a heavy chain with at least 85%, 90%, 95%, 97%, 98%, or 99% of sequence identity after optimal alignment with SEQ ID NO:74.

The following methods may be used to measure the effectiveness of the antibodies and/or antigen binding fragment(s) thereof disclosed herein in the treatment of scleroderma.

### Measures of Scleroderma

### Proposed Phase I Clinical Trial of Teprotumumab for the Treatment of Diffuse Cutaneous Systemic Sclerosis.

Diffuse cutaneous systemic sclerosis (dcSSc) is a multisystem vascular and autoimmune disease resulting in extensive fibrosis of the skin and other organs. Organ-specific fibrosing diseases such as glomerulosclerosis, hypertrophic scars and pulmonary fibrosis, fibrosis occurs in multiple organs in dcSSc Immune perturbations and vascular injury precede and contribute to the development of fibrosis, which, in turn, further exacerbates vascular and immune damage (Asano, 2015; Bhattacharyya, 2011; Volkmann, 2019; Varga, 2007). There is no clear understanding of the initial disease triggers but it is generally accepted that genetic and/or environmental factors cause an injury to the vasculature leading to a complex pathogenesis involving immune activation, inflammation, small vessel damage and an increase in the synthesis and deposition of extracellular matrix components resulting in multiorgan fibrosis (Asano 2015, Asano, 2017). This complex pathogenesis includes but is not limited to activation of dermal fibroblasts, skewing of T helper populations to a Th2/Th17 phenotype, differentiation of macrophages to an M2 phenotype, increased infiltration of plasmacytoid dendritic cells, endothelial-to-mesenchymal transition, epithelial cell activation and differentiation of various cell types into myofibroblasts (Asano, 2017).

Insulin-Like Growth Factor-1 Receptor (IGF-1R):

The insulin-like growth factor-1 receptor (IGF-1R) is a tyrosine kinase cell surface receptor that shares approximately 60% overall homology with the insulin receptor (IR) (Schumacher, 1991). When activated by its ligands, insulin-like growth factor (IGF)-1 and IGF-2, IGF-1R regulates important cellular activities involving cell proliferation, differentiation, and inflammation (Khandwala, 2000, Li, 2018, Ullrich, 1986).

Increasing evidence provides support to the role of the IGF-1/IGF-1R pathway in the pathogenesis of dcSSc including the presence of elevated levels of IGF-1 and associated binding proteins in serum and skin of diseased individuals, ability of IGF-1R stimulation to cause fibroblast migration, proliferation, and differentiation into myofibroblasts, and the requirement for IGF-1R signaling for M2 macrophage polarization. Additionally, preclinical evidence in mice supports a role for the IGF-1R receptor in lung fibrosis following injury. For example:

1. IGF-1 protein levels as well as protein levels of one of its binding partners, IGFBP-3, are elevated in the serum of patients with dcSSC as compared with healthy controls and patients with Systemic Lupus Erythematosus (SLE) or Limited cutaneous systemic sclerosis (lcSSc) (Hamaguchi, 2008).
2. Ribonucleic Acid (RNA) levels of IGF-1 and another binding partner, Insulin like growth factor binding protein (IGFBP-5), have also been shown to be elevated in skin fibroblasts derived from SSc patients (Feghali, 1999; Hamaguchi, 2008).
3. IGF-1 RNA and protein levels were found to be elevated in the skin and serum of patients with Morphea, a chronic disorder with sclerotic plaques and increased fibrosis (Fawzi, 2008)).
4. Case reports where treatment with an IGF-1 antagonist, octreotide, resolved either refractory pretibial myxedema with Graves' disease or skin sclerosis in a patient with carcinoid syndrome (Shinohara, 2000; Pavlovic, 1995).
5. In vitro studies have demonstrated that IGF-1 stimulated differentiation of fibroblasts into myofibroblasts (Hung, 2013).
6. In animal models of acute lung injury, IGF-1R blockade following injury increases survival and decreases time for fibrosis resolution, hydroxyproline content and the number of myofibroblasts, alpha smooth muscle actin (□SMA) expressing cells, in the lungs (Hung, 2013; Choi, 2009).
7. In a model of bleomycin induced lung injury, mice with conditional IGF-1R deletion in lungs showed reduced mortality, reduced alveolar damage, reduced erythrocytes, neutrophils, macrophages and lymphocytes in bronchoalveolar lavage fluid (BALF) as well as prevented vascular permeability changes (Pineiro-Hermida, 2017).
8. IGF-1R has also been shown to be important in M2 macrophage polarization. IGF-1 is highly expressed in M2 macrophages as compared to M0/M1 macrophages (Spadaro, 2017). IGF-1R has also been shown to influence the macrophage activation process as mice with the IGF-1R gene knocked out in cell of the myeloid lineage showed decreased ability to induce the M2 polarization process, reduced transcripts associated with an M2 phenotype as well as an increase in responsiveness to IFN□, a phenotype normally observed in M1 macrophages (Barret, 2015; Spadaro, 2017).

Teprotumumab:

Teprotumumab, a fully human monoclonal antibody (mAb), is an insulin-like growth factor-1 receptor (IGF-1R) inhibitor developed for the treatment of thyroid eye disease (TED).

In vitro, IGF-1R antagonists have shown the ability to block signaling through multiple signal transduction pathways (Akt, MAPK, ERK, etc.), decrease the expression of cytokines and reduce secretion of disease related GAGs (Pritchard, 2003; Tsui, 2008; Smith and Hoa, 2004; Chen, 2014). TED and dcSSc have common disease features including activation of fibroblasts, elevated levels of inflammatory cytokines, infiltration of immune cells into disease tissue and excessive synthesis of extracellular matrix components (Bahn, 2010; Boschi, 2005; Smith, 2010) By blocking signaling and down-regulating IGF-1R in fibroblasts, myofibroblasts, fibrocytes and cells of the immune system, teprotumumab has the potential to specifically resolve the key underlying pathophysiology of dcSSc, thereby reducing the severity and progression of the disease.

Objectives:

Objectives of this study include:
to evaluate the safety, tolerability and effect on insulin-like growth factor-1 (IGF-1), inflammatory and fibrotic biomarkers of teprotumumab, a fully human monoclonal antibody (mAb) inhibitor of the IGF-1 receptor (IGF-1R), administered once every 3 weeks (q3W) for 24 weeks in the treatment of subjects with diffuse cutaneous systemic sclerosis (dcSSc).
to evaluate the safety of teprotumumab versus placebo on the proportion of subjects who experience a treatment-emergent adverse event (TEAE) through Week 24 in subjects with dcSSc.
to evaluate the effect of teprotumumab versus placebo on the responder rate from Baseline to Week 24 in the change in American College of Rheumatology-Composite Response Index in Systemic Sclerosis (ACR-CRISS).
to evaluate the effect of teprotumumab versus placebo on the components of the ACR-CRISS, including modified Rodnan skin score (mRSS), forced vital capacity (FVC) % predicted, Patient Global Assessment (PTGA), Physician Global Assessment (MDGA) and Health Assessment Questionnaire-Disability Index (HAQ-DI) from Baseline to Week 24.
to evaluate the effect of teprotumumab versus placebo on changes from Baseline to Week 24 for flare of disease using components of the ACR-CRISS.
To evaluate the proportion of subjects who improve by ≥20% (≥5% for FVC % predicted) in 3 of 5 core ACR-CRISS items at Week 24.
to evaluate the change from Baseline over time through Week 24 in transcriptomics associated with the IGF-1 pathway, inflammation and fibrosis in skin (lesional) biopsies.
to evaluate the change from Baseline over time through Week 24 in secreted proteins associated with the IGF-1 pathway in serum.
to evaluate the change from Baseline over time through Week 24 in protein expression in skin (lesional) biopsies for IGF-1R and markers of fibrosis and inflammation.
to evaluate the change from Baseline over time through Week 24 in IGF-1R protein expression on peripheral blood mononuclear cells (PBMCs).
to evaluate the effect of teprotumumab on the mean change from Baseline to Week 24 in erythrocyte sedimentation rate (ESR).
to evaluate the effect of teprotumumab on the mean change from Baseline to Week 24 in high sensitivity C-reactive protein (hsCRP).
To evaluate the effect of teprotumumab on the change over time through Week 24 in histology on skin (lesional) biopsies.
To evaluate the effect of teprotumumab on the change from Baseline to Week 24 and from Week 24 to Week 48 in quality-of-life (QOL) measures (Hand Disability in Systemic Sclerosis-Digital Ulcers [HDISS-DU®] and UCLA Scleroderma Clinical Trial Consortium Gastrointestinal Tract [UCLA SCTC GIT 2.0]).

Optionally, the pharmacokinetics (PK) and immunogenicity of teprotumumab will also be evaluated. To assess safety and tolerability of teprotumumab based on TEAEs, adverse events of special interest (AESI) (hyperglycemia, hearing impairment, infusion reaction, new onset or exacerbation of inflammatory bowel disease), concomitant medication use, vital signs, clinical safety laboratory evaluations and inflammatory laboratory evaluations.

Study Design:

This is a randomized, double-blind, placebo-controlled, repeat-dose, multicenter study. Subjects will be screened for the study within 4 weeks prior to the Baseline (Day 1) Visit. Approximately 25 subjects who meet the study eligibility criteria will be randomized on Day 1 in a 3:2 ratio to receive 8 infusions of teprotumumab (10 mg/kg for the first infusion and 20 mg/kg for the remaining 7 infusions) or placebo q3W. During the 24-week double-blind Treatment Period, study drug will be infused on Day 1 (Baseline) and Weeks 3, 6, 9, 12, 15, 18 and 21 with a comprehensive visit at Week 24 (end of treatment). All study drug dosing will be performed at the clinic or infusion center under the supervision of clinic staff or nurses. At any scheduled infusion, the infusion rate may be reduced or the dose may be interrupted or held based on tolerability (see Section 9.4.6.3.2 for details). On each dosing day, scheduled assessments (except for adverse event [AE] and concomitant medication use monitoring, which will be monitored throughout the clinic visit) will be completed prior to study drug infusions. After each of the first 2 infusions, subjects will be contacted by phone/email the following day. Additional phone/email contacts and clinic visits may also be conducted for any subject experiencing an infusion-associated event.

At the end of the Treatment Period (Week 24), subjects will enter a 24-week Follow-up Period, during which study drug will not be administered and a clinic visit will be scheduled for Weeks 28, 36 and 48. A phone call or email at Weeks 32 and 42 will occur to inquire how the subject is doing and women of childbearing potential will be asked if they have missed a menstrual cycle and will have a serum pregnancy test, if required.

Subjects who prematurely discontinue prior to completing the Treatment Period will return to the clinic and undergo the scheduled Week 24 assessments; such subjects will also be encouraged to continue in the 24-week Follow-up Period. An overview of the study design is presented in the schematic below and details of study activities are provided in Section 2.1, Schedule of Assessments.

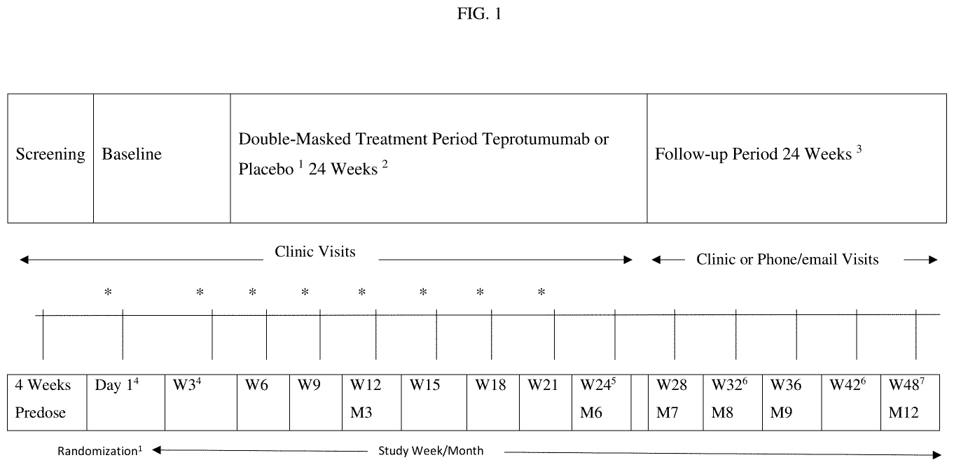

In FIG. 1: * indicates infusion of study drug; AE=adverse event; M=Month; q3W=every 3 weeks; W=Week; 1) subjects will be randomized in a 3:2 ratio (15 Teprotumumab and 10 placebo) to receive: a) teprotumumab (10 mg/kg on Day 1 followed by 20 mg/kg q3W for the remaining 7 infusions); or b) Placebo (placebo q3W for all 8 infusions); 2) visit windows are ±3 days for Weeks 3 to 21, inclusive; 3) visit windows are ±7 days for Weeks 24 to 48, inclusive; 4) all subjects will be contacted by phone/email the day following the first (Day 1) and second (Week 3) infusions for safety and tolerability assessments; additional phone/email contacts will occur the day after any clinic visit where a subject experiences an infusion-related AE; 5) if a subject prematurely discontinues prior to completing the 24-week Treatment Period, he/she will return to the clinic and undergo the Week 24 assessments. Subjects will be encouraged to continue in the 24-week Follow-up Period; 6) all subjects will be contacted via phone or email at Weeks 32 and 42 to inquire how subjects are doing and to ask women of childbearing potential about their menstrual cycle; and 7) if a subject prematurely discontinues prior to completing the 24-week Follow-up Period, he/she will return to the clinic and undergo the Week 48 assessments.

Subject Population:

Approximately 20-25 male and non-pregnant female subjects between the ages of 18 and 80 years, inclusive, with Diffuse Cutaneous Systemic Sclerosis (dcSSc) will be enrolled.

Inclusion Criteria:

Eligible subjects must meet/provide all of the following criteria:

1. Written informed consent.
2. Male or female subject between the ages of 18 and 80 years, inclusive, at Screening.
3. Subject must meet the 2013 American College of Rheumatology (ACR)/European League Against Rheumatism (EULAR) classification criteria for scleroderma/SSc with a total of ≥9.
4. Subjects classified as having skin involvement proximal to elbow, knee, face and neck (dcSSc subset by LeRoy and Medsger, 2001).
5. Subjects with documented active disease by domain as determined by the site investigator, based on data available to the investigator through medical history and/or medical records,
a. worsening of sclerodermatous skin involvement in one or more body areas (including any new areas of involvement) within the last 6 months prior to the Screening Visit,
b. and/or the presence of a tendon friction rub at screening
c. and/or no improvement in sclerodermatous skin involvement (defined as an improvement of >3 units) within the last 6 months prior to the Screening Visit.
   6. At the time of enrollment, no more than 60 months must have elapsed since the onset of the first dcSSc manifestations, other than Raynaud's phenomenon.
   7. Subject must have skin thickening from dcSSc in the forearm suitable for repeat biopsy.
   8. Subject must have a mRSS units ≥10 and ≤45 at screening.
   9. Subjects will be allowed to take CellCept® (mycophenolate mofetil) up to 3 g/day or Myfortic® (mycophenolic acid) up to 2.14 g/day and low-dose prednisone (≤10 mg/day or equivalent dosing of glucocorticoids). Subjects taking CellCept or Myfortic have been doing so for ≥20 weeks and the dose must have been stable for ≥16 weeks prior to the Day 1 Visit. Prednisone must have been at a stable dose for ≥4 weeks prior to the Day 1 Visit.
   10. Diabetic subjects must have glycated hemoglobin (HbA1c)≤8.0%, with no new diabetic medication (oral or insulin) or more than a 10% change in the dose of a currently prescribed diabetic medication within 60 days prior to Screening.
   11. Women of childbearing potential (including those with an onset of menopause <2 years prior to Screening, nontherapy-induced amenorrhea for <12 months prior to Screening or not surgically sterile [absence of ovaries and/or uterus]) must have a negative serum pregnancy test at Screening and negative urine pregnancy tests at all protocol-specified timepoints (i.e., prior to each dose and throughout the subject's participation in the Follow-up Period); subjects who are sexually active with a non-vasectomized male partner must agree to use 2 reliable forms of contraception during the trial, one of which is recommended to be hormonal, such as an oral contraceptive. Hormonal contraception must be started at least one full cycle prior to Baseline and continue for 180 days after the last dose of study drug. Highly effective contraceptive methods (with a failure rate <1% per year), when used consistently and correctly, include implants, injectables, combined oral contraceptives, some intrauterine devices, sexual abstinence or vasectomized partner.
   12. Male subjects must be surgically sterile or, if sexually active with a female partner of childbearing potential, must agree to use a barrier contraceptive method from Screening through 180 days after the last dose of study drug.
   13. Subject is willing and able to comply with the prescribed treatment protocol and evaluations for the duration of the study.

Exclusion Criteria:

Subjects will be ineligible for study participation if they meet any of the following criteria:

1. Subject is diagnosed with limited cutaneous SSc or sine scleroderma.
2. Subject is diagnosed with other autoimmune diseases except for fibromyalgia, scleroderma associated myopathy, and Sjogren's syndrome.
3. Subject must not have had scleroderma renal crisis (SRC) within 6 months of the screening visit. SRC is defined as abrupt onset of hypertension and acute kidney injury.
4. Forced vital capacity (FVC)<50% predicted, diffusing capacity for carbon monoxide (DLCO)<40% predicted, pulmonary arterial hypertension (PAH) by right heart catheterization requiring treatment with more than one oral PAH approved therapy or parental therapy. Intermittent use of PDE-5 inhibitors are allowed for erectile dysfunction and/or Raynaud's Phenomenon/digital ulcers.
5. Corticosteroid use for conditions other than dcSSc within 4 weeks prior to Screening (topical steroids for dermatological conditions and inhaled steroids are allowed).
6. Previous treatment with rituximab (Rituxan® or MabThera®) within 12 months prior to the first infusion.
7. Use of any other non-steroid immunosuppressive agent, cytotoxic or anti-fibrotic drug within 4 weeks of screening other than anti-malarial. This includes cyclophosphamide, azathioprine (Imuran), methotrexate or other immunosuppressive or cytotoxic medication except mycophenolate mofetil or mycophenolic acid [Myfortic].
8. Use of biologics or small molecules approved for rheumatoid arthritis, psoriatic arthritis and other rheumatic diseases within 4 weeks prior to Screening.
9. Use of an investigational agent for any condition within 90 days or 5 half-lives, whichever is longer, prior to Screening or anticipated use during the course of the trial.
10. Malignant condition in the past 5 years (except successfully treated basal/squamous cell carcinoma of the skin or cervical cancer in situ).
11. Pregnant or lactating women.
12. Current drug or alcohol abuse, or history of either within the previous 2 years, in the opinion of the Investigator or as reported by the subject.
13. Biopsy-proven or clinically suspected inflammatory bowel disease (e.g., diarrhea with or without blood or rectal bleeding associated with abdominal pain or cramping/colic, urgency, tenesmus, or incontinence for more than 4 weeks without a confirmed alternative diagnosis OR endoscopic or radiologic evidence of enteritis/colitis without a confirmed alternative diagnosis).
14. Known hypersensitivity to any of the components of teprotumumab or prior hypersensitivity reactions to mAbs.
15. Previous enrollment in this study or participation in a prior teprotumumab clinical trial.
16. Human immunodeficiency virus, untreated or positive viral load for hepatitis C or hepatitis B infections.
17. Previous organ transplant (including allogeneic and autologous marrow transplant).
18. Alanine aminotransferase (ALT) or aspartate aminotransferase (AST) >2.5 times the upper limit of normal (ULN) or eGFR of <30 ml/min/1.73 m² at screening.

### 19. Platelets <120×10⁹/L.

20. Hemoglobin less than 10 g/dL.
21. Any other condition that, in the opinion of the Investigator, would preclude inclusion in the study.

Dose Regimen/Route of Administration:

All study drug dosing will be performed at the clinic under the supervision of clinic staff. On Day 1 of the double-blind Treatment Period, subjects will be randomized to in a 3:1 or 3:2 ratio to receive infusions of either: Teprotumumab 20 mg/kg (10 mg/kg on Day 1 followed by 20 mg/kg q3W for the remaining 7 infusions), or Placebo (q3W for all 8 infusions).

The infusion rate may be reduced and the dose may be interrupted or held based on tolerability. The first and second infusions will be administered over approximately 90 minutes (but not less than 80 minutes). Subsequent infusions will be administered over approximately 60 minutes (but not less than 50 minutes), providing there are no significant infusion-associated events.

Dosage Form and Strength Formulation:

Teprotumumab 500 mg will be provided in single-dose 20 mL glass vials as a freeze-dried powder. Each vial of teprotumumab will be reconstituted with 10 mL of sterile water for injection. The resulting solution will have an approximate concentration of 50 (e.g., 47.6) mg/mL teprotumumab. Reconstituted teprotumumab solution will be further diluted in 0.9% (w/v) sodium chloride (NaCl) solution prior to administration.

Doses up to 1800 mg will be administered in a total infusion volume of 100 mL, and those above 1800 mg will be administered in a total infusion volume of 250 mL. To maintain a constant volume in the infusion bags, a volume equal to the volume of teprotumumab to be placed into the infusion bag will be first removed from the infusion bag using a sterile syringe and needle. The appropriate volume of reconstituted drug product solution based on the subject's dose and body weight will be withdrawn and the teprotumumab reconstituted drug product solution will be diluted with normal saline (0.9% NaCl) in the infusion bag.

Placebo will consist of a normal saline (0.9% NaCl) solution and will be administered in 100 mL or 250 mL infusion bags, as appropriate, per weight-based dosing volumes.

Duration of Treatment:

The planned duration of the Treatment Period is 24 weeks (6 months). At Week 24, all subjects will enter a 24-week Follow-Up Period.

Criteria for Evaluation:

Subjects will be evaluated using the ACR-CRISS, an outcome measure for dcSSc. The ACR-CRISS is a 2-step process that assigns a probability of improvement for a subject that ranges from 0.0 (no improvement) to 1.0 (marked improvement). Step 1 will be evaluated at Weeks 3, 6, 9, 12, 15, 18, 21, 24, 36 and 48, at which time the Investigator will assess if a subject has developed new or worsening cardiopulmonary and/or renal involvement due to SSc. Step 2 involves calculating the probability of improvement based on 5 measures including changes in mRSS, FVC % predicted, HAQ-DI, PTGA and MDGA [Khanna, 2016]. The mRSS, HAQ-DI, PTGA and MDGA will be completed on Day 1 and Weeks 12, 24, 36 and 48; the mRSS will also be completed at Week 6. FVC % predicted will be completed at Screening and Weeks 12, 24 and 48. QOL assessments (HDISS-DU and UCLA SCTC GIT 2.0) will be completed on Day 1 and Weeks 24, 36 and 48.

Blood samples for Teprotumumab PK assessment will be collected pre- and post-infusion on Day 1 and Weeks 3, 12 and 18; a single sample will be collected at Weeks 24 and 36.

Blood samples, including PBMCs for analysis of biomarkers of the IGF-1 pathway, will be collected pre-infusion on Day 1 and Weeks 3, 12 and 18; an additional single sample will be collected at Week 24.

Blood samples for immunogenicity testing will be collected pre-infusion on Day 1 and Weeks 3, 12 and 18; a single sample will be collected at Weeks 24, 36 and 48.

A total of five 3-mm biopsies will be taken from the forearm to analyze for transcriptomics as well as protein expression in skin for IGF-1R and markers of fibrosis and inflammation. Two 3-mm biopsies will be obtained pre-infusion on Day 1 and again at the Week 24 Visit. One 3-mm biopsy will be taken pre-infusion at Week 3.

Safety will be assessed via AE and concomitant medication use monitoring, physical examinations, vital signs, clinical safety laboratory evaluations (complete blood count and chemistry [including HbA1c], clinical inflammatory laboratory evaluations (hsCRP and ESR), pregnancy testing (if applicable), a Screening electrocardiogram, and optionally immunogenicity testing.

Statistical Analyses:

The following endpoints may be statistically analyzed:

1. The proportion of subjects who experience a TEAE (as defined by National Cancer Institute Common Terminology Criteria for Adverse Events [NCI-CTCAE] 5.0) through Week 24 in subjects with dcSSc.
2. The responder rate (defined as ACR-CRISS [predicted probability] of at least 0.6) at Week 24.

### 3. The ACR-CRISS at Week 24.

4. The change from Baseline in mRSS at Week 24.
5. The change from Baseline in FVC % predicted at Week 24.
6. The change from Baseline in PTGA at Week 24.
7. The change from Baseline in MDGA at Week 24.
8. The change from Baseline in HAQ-DI at Week 24.
9. The proportion of subjects who improve by ≥20% (≥5% for FVC % predicted) in 3 of 5 core ACR-CRISS items at Week 24.
10. The change from baseline in biomarkers of the IGF-1 pathway, and optionally biomarkers of inflammation and fibrosis.
11. The change from baseline in transcriptomics associated with IGF-1R pathway, inflammation, and fibrosis.
12. The change from baseline in IGF-1R protein expression in skin biopsies and PBMCs.
13. The change from Baseline to Week 24 and from Week 24 to Week 48 in the HDISS-DU.
14. The change from Baseline to Week 24 and from Week 24 to Week 48 in each scale of the UCLA SCTC GIT 2.0 and the total GIT score.
15. PK of teprotumumab.
16. The incidence of ADA and titer levels.
17. The incidence of TEAEs and AESI (hyperglycemia, hearing impairment, infusion reaction, new onset or exacerbation of inflammatory bowel disease).
18. Concomitant medication use
19. Vital signs: change from Baseline at each scheduled visit.
20. Clinical safety laboratory tests: change from Baseline at each scheduled visit.

Statistical Analysis on Efficacy and Safety Parameters

The analysis on (1) will be conducted using safety analysis set, consisting of all subjects who receive at least one dose of study drug (full or partial dose). The number and percentage of subjects who experience a TEAE will be summarized by treatment group.

Efficacy analyses will be conducted using the intent-to-treat (ITT) analysis set, consisting of all subjects randomized to treatment, with select endpoints also summarized by more restrictive analysis sets, including subjects who received a minimum amount of treatment and have available data. Descriptive summaries for observed and change from baseline for ACR-CRISS will be summarized by treatment group and visit.

Analysis of ACR-CRISS, which is the predicted probability of improvement (see Section 9.6.3.2) based on various assessments of efficacy, will be used for efficacy evaluation. A subject with predicted probability of at least 0.6 will be considered improved. The proportion of subjects who have improved based on ACR-CRISS values at Week 24 will be provided with an exact 95% confidence interval around the difference in the proportion between the 2 groups (teprotumumab minus placebo). In addition, the descriptive statistics (n, mean, standard deviation, median, maximum and minimum) of the predicted probabilities will also be provided by treatment group at each scheduled assessment visit. Descriptive summaries of the individual components of ACR-CRISS for observed and change from Baseline will also be provided.

Using ACR-CRISS data, the number and percentage of subjects with flares will be summarized by treatment group. Flare is defined as one of the following:

1. Worsening of skin disease, defined by an increase of mRSS by >3 units and new symptoms such as pruritus, redness, allodynia or new areas of skin involvement reported by subject.
2. Worsening of HAQ-DI by >=0.500 units or patient global assessment (PTGA) by >=3 [0-10 Scale].
3. Worsening of lung function, defined by decline in FVC by >=10% [absolute change] and symptoms of increase dyspnea
4. New organ involvement such as ACR-CRISS step 1: new PAH, left heart failure with left ventricular ejection fraction (LVEF)<45%, worsening interstitial lung disease (ILD) or new scleroderma renal crisis (SRC).

The number and percentage of subjects with flares will be summarized by treatment group.

Descriptive summaries for observed and change from baseline in IGF-1R pathway, inflammatory and fibrotic biomarkers will be summarized by treatment group at each scheduled visit.

Descriptive summaries for observed and change from baseline in transcriptomic associated with IGF-1R inhibition will be summarized by treatment group at each scheduled visit.

Quality of life data will be analyzed using the intent-to-treat analysis set.

Descriptive summaries of observed and change from Baseline values for the HDISS-DU and UCLA SCTC GIT 2.0 and their components will be summarized by treatment group and visit.

Safety analyses will be performed using the safety analysis set.

The number and percentage of subjects in each treatment group reporting at least one occurrence of a TEAE, a TEAE of grade 3 or higher, a serious TEAE, a TEAE related to study drug, an AESI and a TEAE resulting in discontinuation of treatment will be summarized by treatment group. TEAEs will additionally be summarized by system organ class and preferred term.

Concomitant medications will be summarized by Anatomical Therapeutic Chemical (ATC) Level 4 term and preferred term (PT) using counts and percentage of subjects for each treatment group.

Descriptive summaries of observed and change from Baseline values will be presented for each vital sign parameter by treatment group and visit. A shift table for vital signs by NCI-CTCAE grade and visit will be summarized by treatment group.

Safety laboratory assessments (hematology, coagulation and chemistry [including HbA1c]) and change from Baseline (if applicable) will be summarized by visit and treatment group using descriptive statistics. The laboratory values will be categorized as low, normal and high based on their normal ranges. Shift tables using categories of low, normal and high from Baseline to each visit will be summarized by treatment group. Additionally, a shift table for glucose by NCI-CTCAE grade and visit will be summarized by treatment group. Summaries will be provided separately for hyperglycemia.

Modified Rodnan Skin Score

Measurement of the degree of hardness of the skin by the modified Rodnan total skin score (mRSS or mRTSS). This method uses palpation of the skin in 17 areas of the body and providing a score between zero and three for each area. Zero refers to no thinkening, one is mild thinkening, two is moderate thickening, and three is severe thickening of the skin. A typical mRSS score for a scleroderma patient falls between 16-27.

Results.

Teprotumumab is expected to have efficacy in the treatment of diffuse cutaneous systemic sclerosis (dcSSc) and related conditions including other forms of scleroderma, as well as idiopathic pulmonary fibrosis and interstitial lung disease. This includes those forms of scleroderma specifically excluded from the clinical study above, such as limited cutaneous SSc and sine scleroderma. Teprotumumab is expected to have efficacy in various symptoms and measures of scleroderma and dcSSc, including: Improved ACR-CRISS measures, such as improved modified Rodnan skin score (mRSS), improved forced vital capacity (FVC) % predicted, improved Patient Global Assessment (PTGA), improved Physician Global Assessment (MDGA) and improved Health Assessment Questionnaire-Disability Index (HAQ-DI); reduced incidence and/or severity of flare of disease (e.g., using components of the ACR-CRISS); resulting in an altered transcriptome associated with the IGF-1 pathway, e.g., more closely resembling or associated with a non-diseases phenotype; reduced inflammation and/or fibrosis in skin biopsies (lesional); resulting in altered secreted proteins associated with the IGF-1 pathway, e.g., more closely resembling or associated with a non-diseases phenotype; reduced inflammation and/or fibrosis in serum; altered IGF-1R protein expression on Peripheral Blood Mononuclear Cells (PBMC); reduced erythrocyte sedimentation rate (ESR); and reduced high sensitivity C-Reactive Protein (hsCRP).

With the many similarities in the causes of fibrosis between dcSSc and many ILD's including IPF, it would be likely that teprotumumab would work for the treatment of ILD's including IPF. The above protocol could be modified to test ILD's including IPF. Testing endpoints would be modified and could include FVC, 6MWT, DLCO, and St. George's Respiratory Questionnaire (SGRQ).

### Other Embodiments

The detailed description set-forth above is provided to aid those skilled in the art in practicing the present disclosure. However, the disclosure described and claimed herein is not to be limited in scope by the specific embodiments herein disclosed because these embodiments are intended as illustration of several aspects of the disclosure. Any equivalent embodiments are intended to be within the scope of this disclosure. Indeed, various modifications of the disclosure in addition to those shown and described herein will become apparent to those skilled in the art from the foregoing description, which do not depart from the spirit or scope of the present inventive discovery. Such modifications are also intended to fall within the scope of the appended claims.

What is claimed is:

1. A method of treating scleroderma comprising administering to a subject in need thereof a therapeutically effective amount of an insulin-like growth factor-1 receptor (IGF-1R) inhibitor.

2. The method of claim 1, wherein the scleroderma is localized scleroderma.

3. The method of claim 2, wherein the localized scleroderma is morphea scleroderma or linear scleroderma.

4. The method of claim 1, wherein the scleroderma is systemic scleroderma.

5. The method of claim 4, wherein the systemic scleroderma is selected from the group consisting of limited cutaneous systemic scleroderma, systemic sclerosis sine scleroderma, and diffuse cutaneous systemic sclerosis.

6. The method of claim 5, wherein the systemic scleroderma is diffuse cutaneous systemic sclerosis.

7. A method of treating interstitial lung disease (ILD) comprising administering to a subject in need thereof a therapeutically effective amount of an insulin-like growth factor-1 receptor (IGF 1R) inhibitor.

8. The method of claim 7, wherein the ILD is idiopathic pulmonary fibrosis.

9. A method of reducing fibrosis and/or collagen production and/or accumulation in a subject with scleroderma or interstitial lung disease (ILD), comprising administering to said subject a therapeutically effective amount of an insulin-like growth factor-1 receptor (IGF 1R) inhibitor.

10. The method of claim 9, wherein the subject has scleroderma.

11. The method of claim 10, wherein the scleroderma is systemic scleroderma.

12. The method of claim 11, wherein the systemic scleroderma is selected from the group consisting of limited cutaneous systemic scleroderma, systemic sclerosis sine scleroderma, and diffuse cutaneous systemic sclerosis.

13. (canceled)

14. The method of claim 9, wherein reducing fibrosis and collagen production and/or accumulation is measured by the skin elasticity.

15. (canceled)

16. The method of claim 9, wherein reducing fibrosis and collagen production and/or accumulation is measured as an increase in the subject's American College of Rheumatology-Composite Response Index in Systemic Sclerosis (ACR-CRISS) score.

17. (canceled)

18. (canceled)

19. The method of claim 9, wherein reducing fibrosis and collagen production and/or accumulation is measured as an improvement in the subject's lung function.

20. (canceled)

21. (canceled)

22. (canceled)

23. (canceled)

24. The method of claim 1, wherein the IGF-1R inhibitor is an antibody or antigen binding fragment thereof.

25. (canceled)

26. (canceled)

27. (canceled)

28. (canceled)

29. (canceled)

30. (canceled)

31. (canceled)

32. (canceled)

33. The method of claim 24, wherein the antibody, or an antigen binding fragment thereof, comprises a heavy chain comprising CDR1, CDR2, and CDR3 and a light chain comprising CDR1, CDR2 and CDR3, wherein the heavy chain CDR1, CDR2, and CDR3 amino acid sequences and light chain CDR1, CDR2, and CDR3 amino acid sequences are at least 90% identical to (i) the amino acid sequences of SEQ ID NOs: 85-90, respectively; or (ii) the amino acid sequences of SEQ ID NOs: 85, 93, 87, 88, 94, and 90, respectively.

34. (canceled)

35. (canceled)

36. (canceled)

37. The method of claim 24, wherein the antibody is teprotumumab.

38. (canceled)

39. (canceled)

40. (canceled)

41. (canceled)

42. (canceled)

43. (canceled)

44. (canceled)

45. (canceled)

46. The method of claim 24 wherein said IGF-1R inhibitor is chosen from ganitumab, figitumumab, MEDI-573, cixutumumab, dalotuzumab, robatumumab, AVE1642, BIIB022, xentuzumab, istiratumab, linsitinib, picropodophyllin, BMS-754807, BMS-536924, BMS-554417, GSK1838705A, GSK1904529A, NVP-AEW541, NVP-ADW742, GTx-134, AG1024, KW-2450, PL-2258, NVP-AEW541, NSM-18, AZD3463, AZD9362, BI885578, BI893923, TT-100, XL-228, and A-928605.

47.-65. (canceled)

66. The method of claim 46 wherein the antibody is AVE1642.

67.-101. (canceled)


SEQUENCE LISTING (96 sequences)

SEQ ID NO: 1 - protein, 6 aa, Artificial Sequence
  HCDR1 for Dalotuzumab
GGYLWN

SEQ ID NO: 2 - protein, 16 aa, Artificial Sequence
  HCDR2 for Dalotuzumab
YISYDGTNNYKPSLKD

SEQ ID NO: 3 - protein, 8 aa, Artificial Sequence
  HCDR3 for Dalotuzumab
YGRVFFDY

SEQ ID NO: 4 - protein, 16 aa, Artificial Sequence
  LCDR1 for Dalotuzumab
RSSQSIVHSNGNTYLQ

SEQ ID NO: 5 - protein, 7 aa, Artificial Sequence
  LCDR2 for Dalotuzumab
KVSNRLY

SEQ ID NO: 6 - protein, 9 aa, Artificial Sequence
  LCDR3 for Dalotuzumab
FQGSHVPWT

SEQ ID NO: 7 - protein, 447 aa, Artificial Sequence
  Heavy Chain of Dalotuzumab
QVQLQESGPGLVKPSETLSLTCTVSGYSITGGYLWNWIRQPPGKGLEWIGYISYDGTNNYKPSLKDRVTISRDTSKNQFSLKLSSVTAADTAVYYCARYGRVFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 219 aa, Artificial Sequence
  Light Chain of Dalotuzumab
DIVMTQSPLSLPVTPGEPASISCRSSQSIVHSNGNTYLQWYLQKPGQSPQLLIYKVSNRLYGVPDRFSGSGSGTDFTLKISRVEAEDVGVYYCFQGSHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 6 aa, Artificial Sequence
  HCDR1 of Ganitumab
SSNWWS

SEQ ID NO: 10 - protein, 16 aa, Artificial Sequence
  HCDR2 of Ganitumab
EIYHSGSTNYNPSLKS

SEQ ID NO: 11 - protein, 10 aa, Artificial Sequence
  HCDR3 of Ganitumab
WTGRTDAFDI

SEQ ID NO: 12 - protein, 19 aa, Artificial Sequence
  LCDR1 of Ganitumab
ISCRSSQSLLHSNGYNYLD

SEQ ID NO: 13 - protein, 7 aa, Artificial Sequence
  LCDR2 for Ganitumab
LGSNRAS

SEQ ID NO: 14 - protein, 9 aa, Artificial Sequence
  LCDR3 of Ganitumab
MQGTHWPLT

SEQ ID NO: 15 - protein, 448 aa, Artificial Sequence
  Heavy Chain of Ganitumab
QVQLQESGPGLVKPSGTLSLTCAVSGGSISSSNWWSWVRQPPGKGLEWIGEIYHSGSTNYNPSLKSRVTSVDKSKNQFSLKLSSVTAADTAVYYCARWTGRTDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 219 aa, Artificial Sequence
  Light Chain of Ganitumab
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGTHWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 5 aa, Artificial Sequence
  HCDR1 of Xentuzumab
SYWMS

SEQ ID NO: 18 - protein, 15 aa, Artificial Sequence
  HCDR2 of Xentuzumab
SITSYGSFTYADSVK

SEQ ID NO: 19 - protein, 8 aa, Artificial Sequence
  HCDR3 of Xentuzumab
NMYTHFDS

SEQ ID NO: 20 - protein, 13 aa, Artificial Sequence
  LCDR1 of Xentuzumab
SGSSSNIGSNSVS

SEQ ID NO: 21 - protein, 7 aa, Artificial Sequence
  LCDR2 of Xentuzumab
DNSKRPS

SEQ ID NO: 22 - protein, 11 aa, Artificial Sequence
  LCDR3 of Xentuzumab
QSRDTYGYYWV

SEQ ID NO: 23 - protein, 400 aa, Artificial Sequence
  Heavy Chain of Xentuzumab
QVELVESGGGLVQPGGSLRLSCAASGFTFTSYWMSWVRQAPGKGLELVSSITSYGSFTYYADSVKGRFTISRDNSKNTLYLQMNSLRAEDTAVYYCARNMYTHFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDS

SEQ ID NO: 24 - protein, 216 aa, Artificial Sequence
  Light Chain of Xentuzumab
DIVLTQPPSVSGAPGQRVTISCSGSSSNIGSNSVSWYQQLPGTAPKLLIYDNSKRPSGVPDRFSGSKSGTSASLAITGLQSEDEADYYCQSRDTYGYYWVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 25 - protein, 5 aa, Artificial Sequence
  HCDR1 of AVE1642
SYWMH

SEQ ID NO: 26 - protein, 17 aa, Artificial Sequence
  HCDR2 of AVE1642
EINPSNGRTNYNEKFKR

SEQ ID NO: 27 - protein, 15 aa, Artificial Sequence
  HCDR3 of AVE1642
GRPDYYGSSKWYFDV

SEQ ID NO: 28 - protein, 16 aa, Artificial Sequence
  LCDR1 of AVE1642
RSSQSIVHSNVNTYLE

SEQ ID NO: 29 - protein, 7 aa, Artificial Sequence
  LCDR2 of AVE1642
KVSNRFS

SEQ ID NO: 30 - protein, 9 aa, Artificial Sequence
  LCDR3 of AVE1642
FQGSHVPPT

SEQ ID NO: 31 - protein, 124 aa, Artificial Sequence
  Heavy Chain ofAVE1642
QVQLQQSGAELVKPGASVKLSCKASGYTFTSYWMHWVKQRPGQGLEWIGEINPSNGRTNYNEKFKRKATLTVDKSSSTAYMQLSSLTSEDSAVYYFARGRPDYYGSSKWYFDVWGAGTTVTVSS

SEQ ID NO: 32 - protein, 113 aa, Artificial Sequence
  Light Chain of AVE1642
DVLMTQTPLSLPVSLGDQASISCRSSQSIVHSNVNTYLEWYLQKPGQSPKLLIYKVSNRFSGVPDRFSGSGSGTDFTLRISRVEAEDLGIYYCFQGSHVPPTFGGGTKLEIKR

SEQ ID NO: 33 - protein, 10 aa, Artificial Sequence
  HCDR1 of Figitumumab
GFTFSSYAMN

SEQ ID NO: 34 - protein, 17 aa, Artificial Sequence
  HCDR2 of Figitumumab
AISGSGGTTFYADSVKG

SEQ ID NO: 35 - protein, 16 aa, Artificial Sequence
  HCDR3 of Figitumumab
DLGWSDSYYYYYGMDV

SEQ ID NO: 36 - protein, 11 aa, Artificial Sequence
  LCDR1 of Figitumumab
RASQGIRNDLG

SEQ ID NO: 37 - protein, 7 aa, Artificial Sequence
  LCDR2 of Figitumumab
AASRLHR

SEQ ID NO: 38 - protein, 9 aa, Artificial Sequence
  LCDR3 of Figitumumab
LQHNSYPCS

SEQ ID NO: 39 - protein, 451 aa, Artificial Sequence
  Heavy Chain of Figitumumab
EVQLLESGGGLVQPGGSLRLSCTASGFTFSSYAMNWVRQAPGKGLEWVSAISGSGGTTFYADSVKGRFTISRDNSRTTLYLQMNSLRAEDTAVYYCAKDLGWSDSYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 40 - protein, 214 aa, Artificial Sequence
  Light Chain of Figitumumab
DIQMTQFPSSLSASVGDRVTITCRASQGIRNDLGWYQQKPGKAPKRLIYAASRLHRGVPSRFSGSGSGTEFTLTISSLQPEDFATYYCLQHNSYPCSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 5 aa, Artificial Sequence
  HCDR1 of Dusigitumab
SYDIN

SEQ ID NO: 42 - protein, 17 aa, Artificial Sequence
  HCDR2 of Dusigitumab
WMNPNSGNTGYAQKFQG

SEQ ID NO: 43 - protein, 11 aa, Artificial Sequence
  HCDR3 of Dusigitumab
DPYYYYYGMDV

SEQ ID NO: 44 - protein, 13 aa, Artificial Sequence
  LCDR1 of Dusigitumab
SGSSSNIENNHVS

SEQ ID NO: 45 - protein, 7 aa, Artificial Sequence
  LCDR2 of Dusigitumab
DNNKRPS

SEQ ID NO: 46 - protein, 12 aa, Artificial Sequence
  LCDR3 of Dusigitumab
ETWDTSLSAGRV

SEQ ID NO: 47 - protein, 446 aa, Artificial Sequence
  Heavy Chain of Dusigitumab
QVQLVQSGAEVKKPGASVKVSCKASGYTFTSYDINWVRQATGQGLEWMGWMNPNSGNTGYAQKFQGRVTMTRNTSISTAYMELSSLRSEDTAVYYCARDPYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 48 - protein, 217 aa, Artificial Sequence
  Light Chain of Dusigitumab
QSVLTQPPSVSAAPGQKVTISCSGSSSNIENNHVSWYQQLPGTAPKLLIYDNNKRPSGIPDRFSGSKSGTSATLGITGLQTGDEADYYCETWDTSLSAGRVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 49 - protein, 5 aa, Artificial Sequence
  HCDR1 of Cixutumumab
SYAIS

SEQ ID NO: 50 - protein, 16 aa, Artificial Sequence
  HCDR2 of Cixutumumab
GIIPIFGTANYAQKFQ

SEQ ID NO: 51 - protein, 21 aa, Artificial Sequence
  HCDR3 of Cixutumumab
APLRFLEWSTQDHYYYYYMDV

SEQ ID NO: 52 - protein, 11 aa, Artificial Sequence
  LCDR1 of Cixutumumab
QGDSLRSYYAT

SEQ ID NO: 53 - protein, 7 aa, Artificial Sequence
  LCDR2 of Cixutumumab
GENKRPS

SEQ ID NO: 54 - protein, 11 aa, Artificial Sequence
  LCDR3 of Cixutumumab
KSRDGSGQHLV

SEQ ID NO: 55 - protein, 460 aa, Artificial Sequence
  Heavy Chain of Cixutumumab
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARAPLRFLEWSTQDHYYYYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 214 aa, Artificial Sequence
  Light Chain of Cixutumumab
SSELTQDPAVSVALGQTVRITCQGDSLRSYYATWYQQKPGQAPILVIYGENKRPSGIPDRFSGSSSGNTASLTITGAQAEDEADYYCKSRDGSGQHLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPAECS

SEQ ID NO: 57 - protein, 5 aa, Artificial Sequence
  HCDR1 of BIIB022
IYRMQ

SEQ ID NO: 58 - protein, 17 aa, Artificial Sequence
  HCDR2 of BIIB022
GISPSGGTTWYADSVKG

SEQ ID NO: 59 - protein, 11 aa, Artificial Sequence
  HCDR3 of BIIB022
WSGGSGYAFDI

SEQ ID NO: 60 - protein, 10 aa, Artificial Sequence
  LCDR1 of BIIB022
QASRDIRNYN

SEQ ID NO: 61 - protein, 7 aa, Artificial Sequence
  LCDR2 of BIIB022
DASSLQT

SEQ ID NO: 62 - protein, 9 aa, Artificial Sequence
  LCDR3 of BIIB022
QQFDSLPHT

SEQ ID NO: 63 - protein, 120 aa, Artificial Sequence
  Heavy Chain of BIIB022
EVQLLESGGGLVQPGGSLRLSCAASGFTFSIYRMQWVRQAPGKGLEWVSGISPSGGTTWYADSVKGRFTISRDNSKNTLYLQMNSLRAEDTAVYYCARWSGGSGYAFDIWGQGTMVTVSS

SEQ ID NO: 64 - protein, 107 aa, Artificial Sequence
  Light Chain of BIIB022
DIQMTQSPLSLSASVGDRVTITCQASRDIRNYLNWYQQKPGKAPKLLIYDASSLQTGVPSRFGGSGSGTDFSFTIGSLQPEDIATYYCQQFDSLPHTFGQGTKLEIK

SEQ ID NO: 65 - protein, 448 aa, Artificial Sequence
  HCDR1 of Robatumumab
EVQLVQSGGGLVKPGGSLRLSCAASGFTFSSFAMHWVRQAPGKGLEWISVIDTRGATYYADSVKGRFTISRDNAKNSLYLQMNSLRAEDTAVYYCARLGNFYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 214 aa, Artificial Sequence
  HCDR2 of Robatumumab
EIVLTQSPGTLSVSPGERATLSCRASQSIGSSLHWYQQKPGQAPRLLIKYASQSLSGIPDRFSGSGSGTDFTLTISRLEPEDFAVYYCHQSSRLPHTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 10 aa, Artificial Sequence
  HCDR1of Istiratumab
GFMFSRYPMH

SEQ ID NO: 68 - protein, 16 aa, Artificial Sequence
  HCDR2 of Istiratumab
ISGSGGATPYADSVKG

SEQ ID NO: 69 - protein, 13 aa, Artificial Sequence
  HCDR3 of Istiratumab
DFYQILTGNAFDY

SEQ ID NO: 70 - protein, 11 aa, Artificial Sequence
  LCDR1 of Istiratumab
RASQGISSYLA

SEQ ID NO: 71 - protein, 7 aa, Artificial Sequence
  LCDR2 of Istiratumab
AKSTLQS

SEQ ID NO: 72 - protein, 9 aa, Artificial Sequence
  LCDR3 of Istiratumab
QQYWTFPLT

SEQ ID NO: 73 - protein, 720 aa, Artificial Sequence
  Heavy Chain  of Istiratumab
EVQLLQSGGGLVQPGGSLRLSCAASGFMFSRYPMHWVRQAPGKGLEWVGSISGSGGATPYADSVKGRFTISRDNSKNTLYLQMNSLRAEDTAVYYCAKDFYQILTGNAFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQVQLVQSGGGLVQPGGSLRLSCAASGFTFDDYAMHWVRQAPGKGLEWVAGISWDSGSTGYADSVKGRFTISRDNAKNSLYLQMNSLRAEDTALYYCARDLGAYQWVEGFDYWGQGTLVTVSSASTGGGGSGGGGSGGGGSGGGGSSYELTQDPAVSVALGQTVRITCQGDSLRSYYASWYQQKPGQAPVLVIYGKNNRPSGIPDRFSGSTSGNSASLTITGAQAEDEADYYCNSRDSPGNQWVFGGGTKVTVLG

SEQ ID NO: 74 - protein, 214 aa, Artificial Sequence
  Light Chain of Istiratumab
DIQMTQSPSSLSASLGDRVTITCRASQGISSYLAWYQQKPGKAPKLLIYAKSTLQSGVPSRFSGSGSGTDFTLTISSLQPEDSATYYCQQYWTFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 10 aa, Artificial Sequence
  Heavy chain CDR1 of AVE1642 (humanized version of murine antibody EM164)
GYTFTSYWMH

SEQ ID NO: 76 - protein, 10 aa, Artificial Sequence
  Heavy chain CDR2 of AVE1642 (humanized version of murine antibody EM164)
EINPSNGRTN

SEQ ID NO: 77 - protein, 10 aa, Artificial Sequence
  Heavy chain CDR2 of AVE1642 (humanized version of murine antibody EM164)
EINPSNGRTN

SEQ ID NO: 78 - protein, 17 aa, Artificial Sequence
  Heavy chain CDR2 of AVE1642
EINPSNGRTNYNQKFQG

SEQ ID NO: 79 - protein, 124 aa, Artificial Sequence
  VH2 domain of AVE1642
QVQLVQSGAEVVKPGASVKLSCKASGYTFTSYWMHWVKQRPGQGLEWIGEINPSNGRTNYNQKFQGKATLTVDKSSSTAYMQLSSLTSEDSAVYYFARGRPDYYGSSKWYFDVWGQGTTVTVSS

SEQ ID NO: 80 - protein, 123 aa, Artificial Sequence
  VH3 domain of AVE1642
QVQLVQSGAEVVKPGASVKLSCKASGYTFTSYWMHWVKQRPGQGLEWIGEINPSNGRTNYNQKFQGKATLTVDKSSSTAYMQLSSLTSEDSAVYYFARGRPDYYGSSKWYFDVWGQGTTVTVS

SEQ ID NO: 81 - protein, 113 aa, Artificial Sequence
  VL2 domain of AVE1642
DVVMTQTPLSLPVSLGDPASISCRSSQSIVHSNVNTYLEWYLQKPGQSPRLLIYKVSNRFSGVPDRFSGSGAGTDFTLRISRVEAEDLGIYYCFQGSHVPPTFGGGTKLEIKR

SEQ ID NO: 82 - protein, 113 aa, Artificial Sequence
  VL3 domain of AVE1642
DVLMTQTPLSLPVSLGDPASISCRSSQSIVHSNVNTYLEWYLQKPGQSPKLLIYKVSNRFSGVPDRFSGSGAGTDFTLRISRVEAEDLGIYYCFQGSHVPPTFGGGTKLEIKR

SEQ ID NO: 83 - protein, 113 aa, Artificial Sequence
  VL4 domain of AVE1642
DVLMTQTPLSLPVSLGDPASISCRSSQSIVHSNVNTYLEWYLQKPGQSPRLLIYKVSNRFSGVPDRFSGSGAGTDFTLRISRVEAEDLGIYYCFQGSHVPPTFGGGTKLEIKR

SEQ ID NO: 84 - protein, 113 aa, Artificial Sequence
  VL5 domain of AVE1642
DVVMTQTPLSLPVSLGDPASISCRSSQSIVHSNVNTYLEWYLQKPGQSPKLLIYKVSNRFSGVPDRFSGSGAGTDFTLRISRVEAEDLGIYYCFQGSHVPPTFGGGTKLEIKR

SEQ ID NO: 85 - protein, 5 aa, Artificial Sequence
  CDRH1 teprotumumab
SYGMH

SEQ ID NO: 86 - protein, 17 aa, Artificial Sequence
  CDRH1 teprotumumab
IIWFDGSSTYYADSVRG

SEQ ID NO: 87 - protein, 9 aa, Artificial Sequence
  CDRH2 teprotumumab
ELGRRYFDL

SEQ ID NO: 88 - protein, 11 aa, Artificial Sequence
  CDRL1teprotumumab
RASQSVSSYLA

SEQ ID NO: 89 - protein, 7 aa, Artificial Sequence
  CDRL2 teprotumumab
DASKRAT

SEQ ID NO: 90 - protein, 10 aa, Artificial Sequence
  CDRL3 aa teprotumumab
QQRSKWPPWT

SEQ ID NO: 91 - protein, 118 aa, Artificial Sequence
  VH teprotumumab
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSS

SEQ ID NO: 92 - protein, 108 aa, Artificial Sequence
  VL teprotumumab
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 93 - protein, 17 aa, Artificial Sequence
  CDRH2 teprotumumab antibody 2
IIWFDGSSKYYGDSVKG

SEQ ID NO: 94 - protein, 7 aa, Artificial Sequence
  CDRL2 teprotumumab antibody
DASNRAT

SEQ ID NO: 95 - protein, 118 aa, Artificial Sequence
  VH teprotumumab antibody 2
QVQLVESGGGVVQPGRSLRLSCAASGFTFSSYGMHWVRQAPGKGLEWMAIIWFDGSSKYYGDSVKGRFTISRDNSKNTLYLQMNSLRADDTAVYYCARELGRRYFDLWGRGTLVTVSS

SEQ ID NO: 96 - protein, 108 aa, Artificial Sequence
  VL teprotumumab antibody 2
EIVLTQSPATLSLSPGERATLSCRASQSVSSYLAWYQQKPGQAPRLLIYDASNRATGIPARFSGSGSGTDFTLTISSLEPEDFAVYYCQQRSKWPPWTFGQGTKVEIK